(19) United States
(12) Patent Application Publication
(10) Pub. No.: US 2026/0232574 A1
(43) Pub. Date: Aug. 13, 2026

(54) COMPOSITIONS AND METHODS FOR TREATING DYSMENORRHEA

(71) Applicant: Daré Bioscience, Inc., San Diego, CA (US)

(72) Inventors: David Friend, San Diego, CA (US); Jennifer Kiang, San Diego, CA (US); Nicolas J. Pacelli, Cedar Park, TX (US); Mark Walters, San Diego, CA (US)

(21) Appl. No.: 19/150,789

(22) PCT Filed: Jan. 24, 2024
(86) PCT No.: PCT/US24/12836

§ 371 Date: Jul. 24, 2025

Related U.S. Application Data
(60) Provisional application No. 63/481,415, filed on Jan. 25, 2023.

(51) Int. Cl. A61K 9/00; A61F 6/14; A61K 9/06; A61K 31/196; A61K 47/10; A61K 47/36; A61P 15/00; A61P 29/00

(52) U.S. Cl. CPC A61K 9/0034; A61F 6/14; A61K 9/06; A61K 31/196; A61K 47/10; A61K 47/36; A61P 15/00; A61P 29/00

(57) ABSTRACT

The present disclosure generally relates to compositions and techniques for treatment of dysmenorrhea using intravaginal delivery. In some embodiments, a composition such as a gel may be applied to the vagina of a subject that is relatively viscous, for example, with a viscosity at room temperature of at least 800,000 cP. The composition may also contain an active ingredient for treatment of dysmenorrhea (e.g., primary dysmenorrhea), e.g., diclofenac, and/or pharmaceutically acceptable salts thereof. In addition, certain embodiments as described herein are generally directed to techniques for making or using such compositions, kits including such compositions, or the like.

### CROSS REFERENCE TO RELATED APPLICATIONS

The present application claims priority to and the benefit of U.S. Provisional Application No. 63/481,415, filed Jan. 25, 2023, which is incorporated herein by reference in its entirety.

### FIELD

The present disclosure generally relates to compositions and methods for treatment of dysmenorrhea using intravaginal delivery.

### BACKGROUND

In subjects having a vagina, the vagina represents an important route for drug administration, e.g., due to its large surface area and blood supply. A variety of materials potentially can be applied to the vagina for delivery to a subject, including both local and systemic delivery. However, materials applied to the vagina may be expelled relatively quickly, e.g., due to gravity, as well as vaginal discharge. In addition, the mucosal layer within the vagina presents a significant challenge for drug entry.

Primary dysmenorrhea is a common in women and is described as cramping pain in the lower abdomen occurring just before or during menses in the absence of an identifiable pelvic disease. Secondary dysmenorrhea refers to painful menses resulting from pelvic pathology such as endometriosis. Primary dysmenorrhea is the most common gynecologic problem in menstruating women with prevalence rates that may be as high as 90 percent. Primary dysmenorrhea leads to absenteeism from school or work resulting in lost work hours and productivity. Accordingly, improvements in vaginal delivery techniques improved treatment methods are needed.

### SUMMARY

The present disclosure generally relates to compositions and methods for treatment of dysmenorrhea. The compositions are suitable for intravaginal delivery. The subject matter of the present disclosure involves, in some cases, interrelated products, alternative solutions to a particular problem, and/or a plurality of different uses of one or more systems and/or articles.

In one set of embodiments, the composition comprises a poloxamer, a stabilization polymer, and an active ingredient for treating dysmenorrhea. In some cases, the composition has a viscosity at room temperature of at least 800,000 cP.

In another set of embodiments, the composition comprises a poloxamer, a stabilization polymer, and an active ingredient for treating dysmenorrhea. In some cases, the composition is made by a process comprising forming a composition comprising the poloxamer, the stabilization polymer, and the active ingredient, and removing air from the composition.

In yet another set of embodiments, the composition comprises a poloxamer, a stabilization polymer, and an active ingredient for treating dysmenorrhea. In some cases, the composition is made by a process comprising forming a composition comprising the poloxamer, the stabilization polymer, and the active ingredient, and exposing the composition to a pressure of less than 100 mbar (absolute) for at least 30 minutes.

In still another set of embodiments, the composition is a composition for treatment of dysmenorrhea. In some cases, at least 90 wt % of the composition consists essentially of a poloxamer, a stabilization polymer, an active ingredient for treating dysmenorrhea, and water.

In another aspect, the present disclosure is directed to a method. The method, according to one set of embodiments, comprises applying, to a vagina of a subject, a composition comprising a poloxamer, a stabilization polymer, and an active ingredient for treating dysmenorrhea. In certain embodiments, the composition, as applied, has a viscosity of at least 800,000 cP.

Several methods are disclosed herein of administering a subject with a compound for prevention or treatment of a particular condition. It is to be understood that in each such aspect of the disclosure, the disclosure specifically includes, also, the compound for use in the treatment or prevention of that particular condition, as well as use of the compound for the manufacture of a medicament for the treatment or prevention of that particular condition.

In another aspect, the present disclosure encompasses methods of making one or more of the embodiments described herein, for example, a composition as described herein. In still another aspect, the present disclosure encompasses methods of using one or more of the embodiments described herein, for example, a composition for the treatment of as described herein, and other indications.

In some embodiments, the present disclosure provides kits comprising the compositions described herein, where the kit includes an applicator suitable for vaginal application. In some embodiments, the applicator is pre-filled with the compositions described herein. In some embodiments, the applicator is not pre-filled with the compositions described herein.

In some embodiments, the kit includes one or more of instructions for inserting the applicator into the vagina and instructions for treating dysmenorrhea, or for treating menorrhagia by applying the applicator filled with the composition or gel.

In some embodiments, the composition is at a temperature of about 4° C. for pre-filling the applicator.

In some aspects, provided herein is a method including for treating menorrhagia, the method including applying, to a vagina of a subject, a composition comprising a poloxamer, a stabilization polymer, and an active ingredient for treating menorrhagia, wherein the composition, as applied, has a viscosity of at least 800,000 cP. In some embodiments, the active ingredient is diclofenac. In embodiments, the subject has menorrhagia. In some embodiments, the subject is at risk of having menorrhagia. In some embodiments, the subject is human.

In some embodiments, the composition is a gel. In embodiments, the poloxamer includes poloxamer 407. In embodiments, the stabilization polymer is xanthan gum.

In embodiments, the composition has air at no more than 15 vol %.

In embodiments, the method includes applying the composition through an applicator to the vagina.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates the viscosity of a composition as a function of temperature, in accordance with one embodiment as described herein.

FIG. 2 is a graph showing in vitro release test (IVRT) comparison of drug release versus time (SQRT: square root).

FIG. 3 is a graph showing a regression model for release rate versus drug load.

FIG. 4 is a graph showing a Release plot of a 1% gel composition.

FIG. 5 is a graph showing a linear time plot of a 1% gel composition.

FIG. 6 is a graph showing a Release plot of a 3% gel composition.

FIG. 7 is a graph showing a linear time plot of a 3% gel composition.

FIG. 8 is an image showing appearance results for Batch 178003-2201 (1% diclofenac; Left); Batch 178003-2202 (2% diclofenac; Middle); Batch 178003-2203 (3% diclofenac; Right).

FIG. 9 are microscopy images at 200× magnification for Batch 178003-2201 (1% diclofenac; Left); Batch 178003-2202 (2% diclofenac; Middle); Batch 178003-2203 (3% diclofenac; Right). The images indicated that the API (active pharmaceutical ingredient) is angular crystalline rod-shape material with low sphericity. Batches 178003-2202 & 178003-2203 exhibited higher amounts of API agglomeration in the drug product. There was no indication of agglomeration reported in visual observations.

FIG. 10 is a graph showing a thermal viscosity profile comparison.

FIG. 11A is a graph showing the thermal ramp at a 1% gel composition.

FIG. 11B is a table showing the data from the thermal ramp graph of FIG. 11A.

FIG. 12A is a graph showing the thermal ramp at a 2% gel composition.

FIG. 12B is a table showing the data from the thermal ramp graph of FIG. 12A.

FIG. 13A is a graph showing the thermal ramp at a 3% gel composition.

FIG. 13B is a table showing the data from the thermal ramp graph of FIG. 13A.

FIG. 14A is a graph showing drug release (μg/cm²) over time.

FIG. 14B is a graph showing average drug release rate (μg/hr/cm²) as a function of pH.

FIG. 14C is a graph showing the titration curve of 4% diclofenac sodium in water.

FIG. 15A is a graph showing amplitude sweep of a 1% diclofenac gel composition. Amplitude sweep uses controlled strain with increasing amplitude to determine the product's linear viscoelastic region (LVER). The LVER indicates the range in which the test can be carried. The viscoelastic behavior of a sample can be characterized by the storage modulus G′ and the loss modulus G″ in the LVER region. The storage modulus G′ relates to the materials ability to store energy elastically. The loss modulus G″ of a material is the ratio of the viscous component to the stress and is related to the material's ability to dissipate stress through heat.

FIG. 15B is a graph showing amplitude sweep of a 5% diclofenac gel composition.

FIG. 15C is a graph showing amplitude sweep of a placebo gel composition.

FIG. 16A illustrates the viscosity of a 1% diclofenac gel composition as a function of temperature.

FIG. 16B illustrates the viscosity of a 5% diclofenac gel composition as a function of temperature.

FIG. 16C illustrates the viscosity of a placebo gel composition as a function of temperature.

FIG. 17A is a graph showing in vitro release test (IVRT) comparison of drug release versus time (SQRT: square root) in a 1% diclofenac gel composition.

FIG. 17B is a graph showing in vitro release test (IVRT) comparison of drug release versus time (SQRT: square root) in a 5% diclofenac gel composition.

FIG. 18A is a graph showing the mean cumulative amount of diclofenac (μg/cm²) delivered to the receptor solution 24 h post-application of the formulation and following extraction with MeCN and MeOH. Data points represent the cumulative amount of diclofenac from sample treated with the 3% diclofenac vaginal gel. (n=2) Error bars represent standard deviation.

FIG. 18B is a graph showing the total mean (μg) of diclofenac extracted by MeCN and MeOH. One donor. n=2. Error bars represent standard deviation.

FIG. 18C is a table showing the total mean (μg) and mean cumulative amount (μg) of diclofenac delivered to DMEM 24 h post-application of 3% diclofenac vaginal gel formulation.

FIG. 19A is a graph showing percent applied dose of diclofenac (%) delivered to DMEM over 24 h post-application of 3 formulations. (n=5). Error bars represent one standard deviation.

FIG. 19B is a graph showing mean cumulative amount of diclofenac (μg/cm²) delivered to DMEM over 24 h post-application of 3 formulations. (n=5). Error bars represent one standard deviation.

FIG. 19C is a graph showing flux of diclofenac (μg/min/cm²) delivered to DMEM over 24 h post-application of 3 formulations. (n=5). Error bars represent one standard deviation.

FIG. 20 is a graph showing the mean cumulative amount of diclofenac (μg) delivered to the reconstructed vaginal tissues after 24 h of treatment. Error bars represent the standard deviation.

FIG. 21 is a graph showing mean body weight values over the course of the study.

FIG. 22A is a table showing serum Creatine Kinase levels on Day 8 of the dosing phase of the study.

FIG. 22B is a table showing serum Creatine Kinase levels on Day 22 of the recovery phase of the study.

FIG. 23 is a table showing a sample visit schedule for a presumed 28 day menstrual cycle, with the assumption that dysmenorrhea symptoms start prior to the onset of menses. orange=study visits; green=at home activity or activities; blue=(1) control, baseline, no treatment 7-day assessment window and (2) multiple dose treatment 7-day assessment window for preliminary efficacy endpoints (pain scores for pelvic/vaginal pain and low back pain and use of study provided rescue pain medication); yellow=luteal phase prior to menstrual cycle; pink=single dose pk menstrual cycle; grey=multiple dose pk and pd menstrual cycle

FIG. 24 is a table showing the Schedule of Assessments. Abbreviations: AE=adverse event; BP=blood pressure; conmed=concomitant medication; CRU=clinical research unit; LH=luteinizing hormone; (X)=procedure done as indicated by symptoms, signs or investigator discretion.

### DETAILED DESCRIPTION

The present disclosure generally relates to compositions and techniques for vaginal insertion. In some embodiments, a composition such as a gel may be applied to the vagina of a subject that is relatively viscous, for example, with a viscosity at room temperature of at least 800,000 cP. The composition may also contain an active ingredient, e.g., diclofenac, and/or salts thereof. Compositions having such relatively high viscosities may be useful, for example, to prevent the composition from readily exiting the vagina or degrading too quickly. This may, for example, allow the composition to release the active ingredient over a relatively long period of time to the vagina. In some embodiments, such compositions may be prepared by removing air from the composition to increase its viscosity or cause the composition to form a gel, etc. In addition, certain embodiments as described herein are generally directed to techniques for making or using such compositions, kits including such compositions, or the like.

Contrary to compositions for dermal or surface applications, the compositions described herein are formulated for vaginal application (e.g., a mucosal surface). The vaginal epithelium has completely different biophysical properties compared to other skin surfaces. For example, the inner lining of the vagina consists of multiple layers of (squamous) cells. The basal membrane provides the support for the first layer of the epithelium-the basal layer. The intermediate layers lie upon the basal layer, and the superficial layer is the outermost layer of the epithelium. In developing vaginal compositions, additional factors are considered, including viscosity. This is because materials applied to the vagina may be expelled relatively quickly (e.g., due to gravity or vaginal discharge) and thus the viscosity (thickness or stickiness) of the vaginal composition is particularly relevant. Also, the mucosal layer within the vagina presents a unique challenge for drug entry, and the vagina is designed to permit the passage of normal uterine secretions (e.g., flushing of the vaginal discharge). In light of these challenges, developing vaginal compositions presents itself with unique hurdles as compared to developing compositions for other dermal uses. Moreover, advantages of preparing and formulating compositions for vaginal (local) delivery include lower systemic side effects due to lower systemic concentrations of the drug, but equivalent or better symptom relief due to local delivery of the drug to the affected tissue.

One aspect as discussed herein is generally directed to compositions having relatively high viscosities, or resistance to flow or deformation. In some embodiments, such compositions may have viscosities of, for example, at least 800,000 cP, 1 million cP, or more, or other viscosities as discussed in more detail below. Fluids with such relatively high viscosities do not flow easily and may even resist flow due to gravity in some cases.

The compositions may contain a polymer, which can increase the viscosity of the composition. For example, the polymer may include a poloxamer, which may form a gel in some cases. In addition, in some cases, the composition may also contain xanthan gum, e.g., that can also act as a stabilizer or a thickening agent, which may help increase viscosity. The composition may also contain an active ingredient, such as diclofenac, etc., which can be released from the composition over a suitable period of time. The composition may be applied to the vagina or another suitable body cavity of a subject, for example, where release of the active ingredient is desired, e.g., to treat or prevent an indication, such as described herein. More details of these and other compositions, in accordance with various embodiments, are provided below.

In some embodiments, the active ingredient is diclofenac or pharmaceutically acceptable salt thereof. Diclofenac (chemical name: 2-[2-(2,6-dichloroanilino)phenyl]acetic acid) has a molecule weight of 296.1 g/mol and the structure:

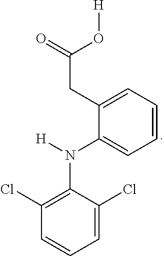 [SMILES: O=C(O)Cc1ccccc1Nc1c(Cl)cccc1Cl]

In certain embodiments, the active ingredient may be present within the composition at concentrations of at least 1 wt %, at least 2 wt %, at least 3 wt %, at least 4 wt %, at least 5 wt %, at least 6 wt %, at least 7 wt %, at least 8 wt %, at least 9 wt %, at least 10 wt %, at least 11 wt %, at least 12 wt %, at least 13 wt %, at least 14 wt %, or at least 15 wt %. In certain embodiments, the diclofenac is present within the composition at a concentration of about 10 wt %. In certain embodiments, the diclofenac is present within the composition at a concentration of about 11 wt %. In certain embodiments, the diclofenac is present within the composition at a concentration of about 12 wt %. In certain embodiments, the diclofenac is present within the composition at a concentration of about 13 wt %. In certain embodiments, the diclofenac is present within the composition at a concentration of about 14 wt %. In certain embodiments, the diclofenac is present within the composition at a concentration of about 15 wt %. In certain embodiments, the diclofenac may be present within the composition at concentrations of about 1 wt %. In certain embodiments, the diclofenac may be present within the composition at concentrations of about 2 wt %. In certain embodiments, the diclofenac may be present within the composition at concentrations of about 3 wt %. In certain embodiments, the diclofenac may be present within the composition at concentrations of about 4 wt %. In certain embodiments, the diclofenac may be present within the composition at concentrations of about 5 wt %. In certain embodiments, the diclofenac may be present within the composition at concentrations of about 6 wt %. In certain embodiments, the diclofenac may be present within the composition at concentrations of about 7 wt %. In certain embodiments, the diclofenac may be present within the composition at concentrations of about 8 wt %. In certain embodiments, the diclofenac may be present within the composition at concentrations of about 9 wt %.

In some embodiments, the composition is administered intravaginally. In some embodiments, the present disclosure provides an applicator that is pre-filled with the composition, where the composition includes a poloxamer, a stabilization polymer, and an active ingredient (e.g., diclofenac or any other therapeutic agent for treating dysmenorrhea or treating primary dysmenorrhea). In some embodiments, the composition includes diclofenac and is used in the treatment of primary dysmenorrhea. Primary dysmenorrhea refers to painful menstruation in the absence of pelvic pathology. Secondary dysmenorrhea refers to painful menses due to pelvic pathology or a recognized medical condition. In some embodiments, the composition includes diclofenac and is used in the treatment of endometriosis. In some embodiments, the composition includes diclofenac and is used in the treatment of generalized pelvic pain.

The present disclosure provides a method, comprising: applying, to a vagina of a subject, a composition comprising a poloxamer, a stabilization polymer, and an active ingredient for treating primary dysmenorrhea. In some embodiments, the active ingredient is diclofenac. In some embodiments, the subject has primary dysmenorrhea. In some embodiments, the subject is at risk of primary dysmenorrhea. In some embodiments, the composition is a gel or hydrogel. In some embodiments, the composition is a hydrogel comprising diclofenac.

In some embodiments, the poloxamer is poloxamer 407. In some embodiments, the stabilization polymer is xanthan gum. In some embodiments, the active ingredient is present at about 1-5 wt %. In some embodiments, the active ingredient is present at about 1-15 wt %. In some embodiments, the composition is administered intravaginally. In some embodiments, the composition is applied as a single dose. In some embodiments, the composition is applied as at least one dose.

Such compositions containing relatively high viscosities may be particularly useful, in certain embodiments, to prevent the composition from readily exiting the vagina (or other cavity), and/or from degrading too quickly after application. This may be used, for example, to assist the delivery of an active ingredient to the subject, e.g., to the vagina of the subject. For instance, fluids with relatively high viscosities may release the active ingredient more slowly and/or uniformly, thereby allowing the active ingredient to be delivered to the subject over a longer period of time. Higher viscosities may also better resist the natural function of the vagina to discharge. Thus, as discussed in more detail herein, the active ingredient may be delivered, e.g., at suitably effective concentrations or amounts, for example, over a period of at least a day, a week, or even longer in some cases. In addition, in some embodiments, only a single dose of the composition may be required to treat a subject, e.g., since the composition does not readily exit the vagina.

As a non-limiting example, in one set of embodiments, a composition may be delivered to a subject having or at risk of dysmenorrhea, menstrual cramps, menorrhagia, and/or pelvic pain. The composition may be applied to the vagina of the subject, and an active ingredient, such as diclofenac, may be delivered to the vagina.

Although other techniques for delivering active ingredients to the vagina (or other body cavity) may also involve the use of relatively high viscosities fluids, these fluids are often selected to have lower viscosities at room temperatures (e.g., about 25° C.) and higher viscosities at body temperatures (e.g., 37° C.). For example, they may be a liquid at room temperature, but become a gel at body temperature. In addition, they may not have viscosities as high as 1 million cP or more; as an example, such a composition may have a viscosity of 300,000 cP at room temperature (and be relatively flowable), increasing to only about 800,000 cP at body temperature.

In contrast, the compositions as discussed herein may have relatively high viscosities, for instance, viscosities as high as 800,000 cP, 1 million cP, 1.5 million cP or more, at room temperature. It should be noted that such compositions, due to their high viscosities, are actually difficult to manufacture; accordingly, most other techniques will use compositions with either lower viscosities, or viscosities that are at least low at room temperatures, typically well below 1 million cP. However, without wishing to be bound by any theory, it is believed that having a high initial viscosity, e.g., upon application to the vagina, may be surprisingly useful for rapid symptom relief. A composition with a relatively high viscosity is less able to lose the active ingredient, i.e., prior to application to the vagina, and thus retains the active ingredient for release to the subject more readily than a liquid or less viscous composition. For example, the composition may at least partially seal the vagina better, e.g., to promote treatment with the active ingredient. In addition, a composition with a relatively high viscosity may itself also stay within the vagina longer. Furthermore, in some embodiments, the presence of the gel itself may be beneficial, i.e., even without the presence of an active ingredient such as diclofenac, etc. Thus, it should be understood that an active ingredient is not always required. Accordingly, such compositions may produce better symptom resolution, e.g., as compared to formulations with relatively low viscosities at room temperature.

Such high viscosities can be achieved, in various embodiments, using techniques such as removing air from the composition, which may increase its viscosity and/or cause it to form a gel. Other techniques may also be used, including any of those described herein. For example, in certain embodiments, a composition may be prepared, e.g., comprising poloxamer, an active ingredient, xanthan gum, water, etc., and the composition treated to remove air from the composition, for example, to reduce the composition to 15 vol % air, or less. Techniques for removing air include, but are not limited to, a variety of techniques, such as centrifugation or exposure to relatively high vacuums, e.g., less than 100 mbar. In addition, it should be understood that the composition need not be limited to those described above, and in other embodiments, other compositions may also be used. Examples of such compositions follow.

For instance, in some aspects, a composition may include one or more poloxamers, xanthan gum, and/or another stabilization polymer, and an active ingredient such as any of those discussed herein. Water may be present in some cases, e.g., such that the composition is a gel, and/or has a relatively high viscosity at room temperature, such as is described herein. Other components may be present as well in certain embodiments, for instance, citrate and/or a citrate salt, benzyl alcohol, or the like. These may act, for example, as excipients, preservatives, antimicrobials, bulking agents, stabilizers, antioxidants, buffers, pH regulating agents, or the like. In addition, in some cases, other components that increase the viscosity of the composition may also be used, for example, hyaluronic acid, alginic acid, modified celluloses such as hydroxypropyl methylcellulose, in addition or instead of poloxamer.

As mentioned, the composition may include one or more poloxamers in one set of embodiments. The poloxamer may be used to increase the viscosity of the composition, e.g., as described herein. In some cases, sufficient poloxamer may be present to cause the composition to form a gel, e.g., at room temperature (about 25° C.) and/or body temperature (about 37° C.). Furthermore, in some cases, the composition may have a gelling temperature, but the gel temperature may be in a range that is physiologically irrelevant. For instance, the gelling temperature may be above 40° C., or below 25° C., and thus, the composition does not change phase or gel at normal physiological or body temperatures.

Poloxamers generally include any of a variety of polyoxyethylene-polyoxypropylene triblock copolymers. In some cases, the poloxamer may be a nonionic block copolymer composed of a central hydrophobic chain of polyoxypropylene (polypropylene oxide) flanked by two hydrophilic chains of polyoxyethylene (polyethylene oxide). In some embodiments, the poloxamers may be soluble in water and other polar and non-polar solvents.

Because the lengths of the polymer blocks can be independently customized, many different poloxamers exist that have slightly different properties. For example, the poloxamer may have a structure:

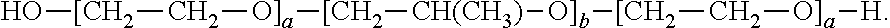

The structure includes a core of propylene oxide (represented by “b” in the above figure), flanked by ethylene oxide subunits (represented by “a” in the above figure), typically on both sides. The sum of the two a's may be, for example, from 50 to 500, from 100 to 300, from 150 to 250, or 200. As another example, a may be between 99 and 103, e.g., 101. In embodiments, b may be, for example, from 30 to 100, from 50 to 80, from 60 to 70, or 65. As another example, b may be between 54 and 58, e.g., 56.

In some embodiments, the ethylene oxide subunits forming the poloxamer may be in molar excess to the propylene oxide subunits. For example, in certain embodiments, the ratio of ethylene oxide subunits to propylene oxide subunits (i.e., a: b) may be, for example, from 3:1 to 5:1, or from 2:1 to 4:1.

Several suitable poloxamers can be readily obtained commercially, including poloxamer 407, Pluronic® F-127, or the like. The composition may include a single poloxamer, or more than one type of poloxamer. In some cases, at least 50 wt %, at least 60 wt %, at least 70 wt %, at least 80 wt %, or at least 90 wt % of the poloxamer within the composition is a single type of poloxamer, for example, poloxamer 407 or Pluronic® F-127.

The molecular weight of the poloxamer may be, in one embodiment, from 5 kDa to 25 kDa. In some instances, the molecular weight of the copolymer may be from 9 kDa to 16 kDa. In some cases, the molecular weight of the poloxamer may be at least 1 kDa, at least 2 kDa, at least 3 kDa, at least 4 kDa, at least 5 kDa, at least 7 kDa, at least 9 kDa, at least 10 kDa, at least 15 kDa, at least 16 kDa, at least 20 kDa, at least 25 kDa, at least 50 kDa, etc. In addition, in certain embodiments, the molecular weight of the poloxamer may be no more than 50 kDa, no more than 25 kDa, no more than 20 kDa, no more than 16 kDa, no more than 15 kDa, no more than 10 kDa, no more than 9 kDa, no more than 5 kDa, no more than 4 kDa, no more than 3 kDa, no more than 2 kDa, no more than 1 kDa, etc. Combinations of any of these are also possible. For instance, the poloxamer may have a molecular weight of between 10 kDa and 15 kDa. As other non-limiting examples, the molecular weight may be between 3 kDa and 5 kDa, between 2 kDa and 4 kDa, between 5 kDa and 20 kDa, between 9 kDa and 16 kDa, etc. The molecular weight, in some cases, may be determined as a weight average molecular weight.

In certain embodiments, the poloxamer may be present within the composition at concentrations of at least 1 wt %, at least 2 wt %, at least 3 wt %, at least 5 wt %, at least 7 wt %, at least 10 wt %, at least 15 wt %, at least 20 wt %, at least 25 wt %, at least 30 wt %, at least 40 wt %, or at least 50 wt %. In addition, in some embodiments, the poloxamer may be present within the composition at concentrations of no more than 50 wt %, no more than 40 wt %, no more than 30 wt %, no more than 25 wt %, no more than 20 wt %, no more than 15 wt %, no more than 10 wt %, no more than 7 wt %, no more than 5 wt %, no more than 3 wt %, no more than 2 wt %, etc. Combinations of any of these are also possible in other embodiments. For example, the poloxamer may be present in a composition at between 10 wt % and 20 wt %, between 5 wt % and 15 wt %, between 15 wt % and 30 wt %, etc.

In one embodiment, the poloxamer used in the composition is Pluronic® F-127. In Pluronic® F-127, the sum of two a's in the above block polymer structure may be 200, and b may have a value of 65. In Pluronic® F-127, the ratio of the sum of two a's to b in the poloxamer (i.e., a: b) may be from 2:1 to 4:1. Tables 1 and 2 illustrate chemical composition and specifications of Pluronic® F-127.

TABLE 1
Chemical Composition of Pluronic ® F-127

| | | | | | | Number average | | |
|---|---|---|---|---|---|---|---|---|
| | | | | Content of | | molecular | Poly | Number of |
| | | | | Oxyethelene | Molecular | weight | dispersity | [symbol] |
| Pluronic | Poloxamer | a | b | (Percent) | Weight | (g/mol) | index | [symbol] |
| [symbol] | 407 | [symbol] | [symbol] | [symbol] | [symbol] | [symbol] | [symbol] | [symbol] |

[symbol]

indicates data missing or illegible when filed

TABLE 2
Specifications of Pluronic ® F-127

| | pH | Cold | | | | | [symbol] | Propylene | [symbol] |
|---|---|---|---|---|---|---|---|---|---|
| Physical | (2.5% | point | APHA | | BET | [symbol] | Oxide | Oxide | [symbol] |
| Form | in water) | [symbol] | [symbol] | [symbol] | ppm | [symbol] | ppm | ppm | ppm |
| Solid | [symbol] | [symbol] | [symbol] | [symbol] | [symbol] | [symbol] | [symbol] | [symbol] | [symbol] |
| | | | max | [symbol] | [symbol] | | | | |

[symbol]

indicates data missing or illegible when filed

In addition, in one set of embodiments, the composition may comprise xanthan gum, and/or another stabilization polymer. Examples of other stabilization polymers include hyaluronic acid, alginic acid, modified celluloses such as hydroxypropyl methylcellulose, or others such as described herein. Xanthan gum generally refers to a high molecular weight polysaccharide used as a food additive and rheology modifier, as would be known by those of ordinary skill in the art. In addition, many such xanthan gums are readily available commercially. Xanthan gum may be produced, as a non-limiting example, by a process involving fermentation of glucose or sucrose by the Xanthomonas campestris bacterium. In some embodiments, the backbone of the polysaccharide chain may have two beta-D-glucose units linked through the 1 and 4 positions. The side chains are formed of two mannose and one glucuronic acid, so the chain has repeating modules of five sugar units. The side chain is linked to every other glucose of the backbone at the 3 position. About half of the terminal mannose units have a pyruvic acid group linked as a ketal to its 4 and 6 positions. The other mannose unit has an acetyl group at the 6 positions. Two of these chains may be aligned to form a double helix, giving a rather rigid rod configuration that accounts for its high efficiency as a viscosifier of water.

However, it should be understood that not all xanthan gums have precisely the above molecular configuration or properties, and that xanthan gums may vary in molecular composition, e.g., depending on the source of the xanthan gum, especially those arising from different biological sources. In addition, other stabilization polymers instead of (or in addition to) xanthan gum can be used, for example, KELTROL® BT and/or KELTROL® RD, KELZAN® XC, KELZAN® XCD, KELZAN® D, KELZAN® CC, XANTURAL® 180, XANTURAL® 75, or the like, all of which can be obtained commercially from various suppliers.

The molecular weight of the xanthan gum or other stabilization polymer can vary. For instance, the xanthan gum or other stabilization polymer may have any suitable molecular weight, for example, at least about 1 million, at least about 2 million, at least about 5 million, at least about 10 million, at least about 25 million, or at least about 50 million. In other embodiments, the molecular weight can vary from about one million to 50 million, e.g., depending upon various factors such as how it is prepared. In some embodiments, the molecular weight can range from approximately 1 million to approximately 25 million, e.g., as measured by a Brookfield Viscometer or other suitable device. In yet other embodiments, the molecular weight may be, for example, 1, 2, 3, 4, or 5 (+/−0.5) million, or 6, 7, 8, 9, 10, 11, 12, 13, 14, 15, 16, 17, 18, 19, 19, 20, 21, 22, 23, 24, or 25 (+/−2) million. Still other molecular weights are also possible.

The xanthan gum (and/or another stabilization polymer) may be present within the composition at concentrations of at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, etc. In addition, in some cases, the xanthan gum and/or other stabilization polymer may be present at no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the xanthan gum and/or other stabilization polymer may be present at between 1 wt % and 5 wt %, between 0.5 wt % and 2 wt %, between 0.5 wt % and 5 wt %, between 0.5 wt % and 2 wt %, or the like.

In one set of embodiments, the composition may also comprise an active ingredient. The active ingredient may be one suitable for treating any of the conditions described herein. For instance, the active ingredient may be one suitable for treatment of a subject for a condition when the active ingredient is delivered to the vagina, or another suitable body cavity. In some embodiments, the active ingredient is present in a therapeutically effective amount. One or more than one active ingredient may be used, depending on the embodiment.

In another set of embodiments, the active ingredient may be one that is suitable to treat a subject having or at risk of dysmenorrhea or menstrual cramps. In some cases, a composition such as is described herein may be inserted into the vagina of a subject. An active ingredient may be released from the composition to treat the subject. In one embodiment, for example, the composition may contain diclofenac and/or a salt thereof as an active ingredient, e.g., diclofenac potassium, diclofenac sodium, etc.

In another set of embodiments, the active ingredient may be an ingredient that can be delivered to the vaginal, e.g., for local or systemic delivery to the subject. In some embodiments, the active ingredient may be any active ingredient that can be dissolved and/or suspended within a composition as described herein, for example, a gel. For example, the active ingredient may be one that is at least partially water soluble. The gel may be relatively viscous, e.g., as discussed herein.

If a salt is present, the salt may be a pharmaceutically acceptable salt in some embodiments. Pharmaceutically acceptable salts include salts which are, within the scope of sound medical judgment, suitable for use in contact with the tissues of a subject (e.g., a human) without undue toxicity, irritation, allergic response, and the like, and are commensurate with a reasonable benefit/risk ratio. Pharmaceutically acceptable salts may also be salts that are generally safe, non-toxic and neither biologically nor otherwise undesirable and includes that which is acceptable for human pharmaceutical use. Pharmaceutically acceptable salts are well known in the art. Pharmaceutically acceptable salts of the compounds described herein include those derived from suitable inorganic and organic acids and bases. Examples of pharmaceutically acceptable, nontoxic acid salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

In some cases, the active ingredient may be released from the composition over any suitable period of time. For example, a therapeutically effective amount of the active ingredient may be released from 1 to 12 hours. In examples, the active ingredient may be released at therapeutically effective amounts from the composition, following application to the subject, for at least 30 minutes, 60, minutes, 90 minutes, 120 minutes, 180 minutes, 4 hours, 5 hours, 6 hours, 7 hours, 8 hours, 9 hours, or 10 or more hours. In some instances, a therapeutically effective amount of the active ingredient may be released from 1 to 21 days. In some instances, a therapeutically effective amount of the active ingredient may be released up to about 1 to 7 days, about 5 or 15 days, or about 8 to 29 days following administration. As still other examples, the active ingredient may be released at therapeutically effective amounts from the composition, following application to the subject, for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days. In some embodiments, only a single application of the composition to the subject may be needed. However, in other embodiments, the composition may be applied to the subject more than once, for example, in applications separated by any of the lengths of time discussed herein.

For example, in some cases, tₘₐₓ, or the time at which the maximum concentration of the active ingredient is present in the vagina (due to release by the composition) may be at least 1 day, at least 2 days, at least 3 days, etc., or other times such as those described above. In addition, in some embodiments, tₘₐₓ may be less than 30, 29, 28, 27, 26, 25, 24, 23, 22, 21, 20, 19, 18, 17, 16, 15, 14, 13, 12, 11, 10, 9, 8, 7, 6, 5, 4, 3, or 2 days. Combinations of any of these are also possible, e.g., tₘₐₓ may be between 1 and 3 days, or between 4 and 6 days, etc. Without wishing to be bound by any theory, it is believed that this may be due to the relatively high viscosity of the composition and/or slower release kinetics from the composition.

The active ingredient may be present within the composition at any suitable concentration. For example, the active ingredient may be present at at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, at least 10.5 wt %, at least 11 wt %, at least 11.5 wt %, at least 12 wt %, at least 12.5 wt %, at least 13 wt %, at least 13.5 wt %, at least 14 wt %, at least 14.5 wt %, at least 15 wt %, etc. In addition, in some cases, the active ingredient may be present at no more than 15 wt %, no more than 14.5 wt %, no more than 14 wt %, no more than 13.5 wt %, no more than 13 wt %, no more than 12.5 wt %, no more than 12 wt %, no more than 11.5 wt %, no more than 11 wt %, no more than 10.5 wt %, no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the active ingredient may be present at between 1 wt % and 5 wt %, between 0.5 wt % and 2 wt %, between 0.5 wt % and 5 wt %, between 2 wt % and 10 wt %, between 5 wt % and 15 wt %, between 7 wt % and 16 wt %, or the like.

In addition, in accordance with certain embodiments, the composition may release the active ingredient over an extended period of time. In some cases, this may be determined by determining a concentration of an active ingredient in the mucus of the vagina after a certain period of time, for instance, after 1 hour, 2 hours, 3 hours, 1 day, 2 days, 3 days, etc. For example, in one set of embodiments, the concentration of the active ingredient may be at least 100 micrograms/g, at least 200 micrograms/g, at least 300 micrograms/g, at least 400 micrograms/g, at least 500 micrograms/g, at least 600 micrograms/g, at least 700 micrograms/g, etc. The mucus may be sampled, for example, using a swab, or other techniques known to those of ordinary skill in the art.

Other components may be present as well within a composition. As a non-limiting example, in one set of embodiments, the composition may include citrate and/or a citrate salt. These may include, for example, citric acid, citric acid monohydrate, sodium citrate, sodium citrate dihydrate, or the like. Other examples include other suitable salts, e.g., to make citrate buffer such as sodium phosphate, potassium phosphate, or the like. Buffers such as these may be used, for example, to maintain the pH of the composition (for example, at around 4.5, or another suitable pH). As another example, the composition may include benzyl alcohol. Benzyl alcohol may be useful, for example, as a solvent or a preservative.

Components such as these may each independently be present in any suitable amount or concentration. For example, a component may be present at at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, etc. In addition, in some cases, a component may be present at no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the component may be present at between 1 wt % and 5 wt %, between 0.5 wt % and 2 wt %, between 0.5 wt % and 5 wt %, between 0.5 wt % and 2 wt %, or the like.

In addition, in one set of embodiments, water may be present within the composition. Any suitable amount of water may be present, for example, such that the composition forms a gel, has a relatively high viscosity as discussed herein, or the like. For example, in some cases, at least 50 wt %, at least 55 wt %, at least 60 wt %, at least 65 wt %, at least 70 wt %, at least 75 wt %, at least 80 wt %, at least 85 wt %, at least 90 wt %, or at least 95 wt % of the composition may be water.

In one set of embodiments, the composition is a gel. In one set of embodiments, the composition is a hydrogel. The gel or hydrogel may be semi-solid material that includes a relatively large amount or concentration of water, e.g., as noted above. In some cases, the polymer (e.g., one or more poloxamers) may from a scaffold structure that contains the water within the gel.

The gel or hydrogel or other composition, e.g., as described herein, may have a relatively high viscosity, at least in one set of embodiments. Those of ordinary skill in the art will be aware of techniques for determining viscosity of a sample, for example, using devices such as a rheometers, viscometers, etc.

In some cases, the composition may have a viscosity at room temperature of at least 800,000 cP, at least 900,000 cP, at least 1 million cP, at least 1.1 million cP, at least 1.2 million cP, at least 1.3 million cP, at least 1.4 million cP, at least 1.5 million cP, at least 1.6 million cP, at least 1.8 million cP, at least 2 million cP, etc. In addition, in certain embodiments, the composition may have a viscosity of no more than 2.0 million cP, no more than 1.8 million cP, no more than 1.6 million cP, no more than 1.5 million cP, no more than 1.4 million cP, no more than 1.3 million cP, no more than 1.2 million cP, no more than 1.1 million cP, no more than 1.0 million cP, no more than 900,000 cP, no more than 800,000 cP, etc. Combinations of any of these are also possible, for example, the composition may exhibit a viscosity of between 800,000 cP and 1 million cP, between 900,000 cP and 1.1 million cP, between 1 million cP and 1.6 million cP, between 1.1 million cP and 1.8 million cP, etc.

In some cases, the composition may have a viscosity at 5° C. of at least 2,000 cP, at least 5,000 cP, at least 10,000 cP, at least 25,000 cP, at least 50,000 cP, at least 75,000 cP, at least 100,000 cP, at least 125,000 cP, at least 150,000 cP, at least 175,000 cP, at least 200,000 cP, at least 220,000 cP, etc. In addition, in certain embodiments, the composition may have a viscosity of no more than 220,000 cP, no more than 200,000 cP, no more than 175,000 cP, no more than 150,000 cP, no more than 100,000 cP, no more than 75,000 cP, no more than 50,000, no more than 25,000 cP, no more than 10,000 cP, no more than 5,000 cP, no more than 2,000 cP, etc. Combinations of any of these are also possible, for example, the composition may exhibit a viscosity of between 2,000 cP and 50,000 cP, between 10,000 cP and 100,000 cP, between 50,000 cP and 150,000 million cP, between 75,000 cP and 220,000 cP, etc.

In some cases, the composition may have a viscosity at 37° C. of at least 800,000 cP, at least 900,000 cP, at least 1 million cP, at least 1.1 million cP, at least 1.2 million cP, at least 1.3 million cP, at least 1.4 million cP, etc. In addition, in certain embodiments, the composition may have a viscosity of no more than 1.4 million cP, no more than 1.3 million cP, no more than 1.2 million cP, no more than 1.1 million cP, no more than 1.0 million cP, no more than 900,000 cP, no more than 800,000 cP, etc. Combinations of any of these are also possible, for example, the composition may exhibit a viscosity of between 800,000 cP and 1 million cP, between 900,000 cP and 1.1 million cP, between 1 million cP and 1.3 million cP, between 1.1 million cP and 1.4 million cP, etc.

In some cases, the composition may contain xanthan gum and/or another stabilization polymer, and a polymer such as a poloxamer, which may cause the composition to have a relatively high viscosity. In some embodiments, the composition may contain no other component that changes the viscosity of said composition at room temperature by more than +/−100,000 centipoise.

Due to the natural acidity of the vagina, vaginal compositions typically aim for a pH of about 4.5. In embodiments, the pH of the composition may be between about 4 and about 7. In embodiments, the pH of the composition may be between about 4 and about 5.5. In embodiments, the pH of the composition may be between about 4 and about 5. In embodiments, the pH of the composition may be between about 4.5 and about 5.5. For example, the composition may have pH about 4, 4.1, 4.2, 4.3, 4.4, 4.5, 4.6, 4.7, 4.8, or 4.9. In embodiments, vaginal compositions as described herein (e.g., using diclofenac) have up to about pH 5.5, for example pH 5.0, 5.1, 5.2, 5.3, 5.4, or 5.5. In embodiments, vaginal compositions as described herein (e.g., using diclofenac) have up to about pH 7, for example pH 5.6, 5.7, 5.8, 5.9, 6.0, 6.1, 6.2, 6.3, 6.4, 6.5, 6.6, 6.7, 6.8, 6.9, or 7.0.

In embodiments, a composition as described herein may release about 50% of the drug within the composition in about 30 minutes to about 4 hours. In embodiments, a composition as described herein may release about 50% of the drug within the composition in about 1 hour to about 80 hours. In embodiments, a composition as described herein may release about 50% of the drug within the composition in about 10 hours to about 70 hours. In embodiments, a composition as described herein may release about 50% of the drug within the composition in about 20 hours to about 70 hours. In embodiments, a composition as described herein may release about 50% of the drug within the composition in about 10 hours to about 60 hours. In embodiments, a composition as described herein may release about 50% of the drug within the composition in about 10 hours to about 50 hours. In embodiments, a composition as described herein may release about 50% of the drug within the composition in about 10 hours to about 40 hours. In embodiments, a composition as described herein may release about 50% of the drug within the composition in about 10 hours to about 30 hours. In embodiments, a composition as described herein may release about 50% of the drug within the composition in about 15 hours to about 60 hours. In embodiments, a composition as described herein may release about 50% of the drug within the composition in about 15 hours to about 50 hours. In embodiments, a composition as described herein may release about 50% of the drug within the composition in about 15 hours to about 40 hours. In embodiments, a composition as described herein may release about 50% of the drug within the composition in about 15 hours to about 30 hours. In embodiments, a composition as described herein may release about 50% of the drug within the composition in about 20 hours to about 60 hours. In embodiments, a composition as described herein may release about 50% of the drug within the composition in about 20 hours to about 50 hours. In embodiments, a composition as described herein may release about 50% of the drug within the composition in about 20 hours to about 40 hours. In embodiments, a composition as described herein may release about 50% of the drug within the composition in about 20 hours to about 30 hours. The time may be any value or subrange within the recited ranges.

In embodiments, a composition as described herein may release about 80% of the drug within the composition in about 30 minutes to about 4 hours. In embodiments, a composition as described herein may release about 80% of the drug within the composition in about 1 hour to about 80 hours. In embodiments, a composition as described herein may release about 80% of the drug within the composition in about 10 hours to about 70 hours. In embodiments, a composition as described herein may release about 80% of the drug within the composition in about 20 hours to about 70 hours. In embodiments, a composition as described herein may release about 80% of the drug within the composition in about 10 hours to about 60 hours. In embodiments, a composition as described herein may release about 80% of the drug within the composition in about 10 hours to about 50 hours. In embodiments, a composition as described herein may release about 80% of the drug within the composition in about 10 hours to about 40 hours. In embodiments, a composition as described herein may release about 80% of the drug within the composition in about 10 hours to about 30 hours. In embodiments, a composition as described herein may release about 80% of the drug within the composition in about 15 hours to about 60 hours. In embodiments, a composition as described herein may release about 80% of the drug within the composition in about 15 hours to about 50 hours. In embodiments, a composition as described herein may release about 80% of the drug within the composition in about 15 hours to about 40 hours. In embodiments, a composition as described herein may release about 80% of the drug within the composition in about 15 hours to about 30 hours. In embodiments, a composition as described herein may release about 80% of the drug within the composition in about 20 hours to about 60 hours. In embodiments, a composition as described herein may release about 80% of the drug within the composition in about 20 hours to about 50 hours. In embodiments, a composition as described herein may release about 80% of the drug within the composition in about 30 hours to about 60 hours. In embodiments, a composition as described herein may release about 80% of the drug within the composition in about 40 hours to about 50 hours. The time may be any value or subrange within the recited ranges.

In embodiments, a composition as described herein may release about 100% of the drug within the composition in about 30 minutes to about 4 hours. In embodiments, a composition as described herein may release about 100% of the drug within the composition in about 1 hour to about 80 hours. In embodiments, a composition as described herein may release about 100% of the drug within the composition in about 10 hours to about 70 hours. In embodiments, a composition as described herein may release about 100% of the drug within the composition in about 20 hours to about 70 hours. In embodiments, a composition as described herein may release about 100% of the drug within the composition in about 10 hours to about 60 hours. In embodiments, a composition as described herein may release about 100% of the drug within the composition in about 10 hours to about 50 hours. In embodiments, a composition as described herein may release about 100% of the drug within the composition in about 10 hours to about 40 hours. In embodiments, a composition as described herein may release about 100% of the drug within the composition in about 10 hours to about 30 hours. In embodiments, a composition as described herein may release about 100% of the drug within the composition in about 15 hours to about 60 hours. In embodiments, a composition as described herein may release about 100% of the drug within the composition in about 20 hours to about 60 hours. In embodiments, a composition as described herein may release about 100% of the drug within the composition in about 30 hours to about 60 hours. In embodiments, a composition as described herein may release about 100% of the drug within the composition in about 40 hours to about 60 hours. In embodiments, a composition as described herein may release about 100% of the drug within the composition in about 45 hours to about 60 hours. The time may be any value or subrange within the recited ranges.

In embodiments, a composition as described herein may release about 25 μg/cm² to about 235 μg/cm² of the drug in 1 hour. In embodiments, a composition as described herein may release about 65 μg/cm² to about 375 μg/cm² of the drug in 2 hours. In embodiments, a composition as described herein may release about 130 μg/cm² to about 490 μg/cm² of the drug in 3 hours. In embodiments, a composition as described herein may release about 200 μg/cm² to about 585 μg/cm² of the drug in 4 hours. In embodiments, a composition as described herein may release about 280 μg/cm² to about 675 μg/cm² of the drug in 5 hours.

In embodiments, a composition at pH 3.5 as described herein may release about 280 μg/cm² of the drug in 5 hours. In embodiments, a composition at pH 3.5 as described herein may release about 250 μg/cm², about 300 μg/cm², or about 350 μg/cm² of the drug in 5 hours.

In embodiments, a composition at pH 4.5 as described herein may release about 440 μg/cm² of the drug in 5 hours. In embodiments, a composition at pH 4.5 as described herein may release about 400 μg/cm², about 425 μg/cm², about 450 μg/cm², or about 500 μg/cm² of the drug in 5 hours.

In embodiments, a composition at pH 5.5 as described herein may release about 510 μg/cm² of the drug in 5 hours. In embodiments, a composition at pH 5.5 as described herein may release about 500 μg/cm², about 525 μg/cm², about 550 μg/cm², or about 600 μg/cm² of the drug in 5 hours.

In embodiments, a composition at pH 6.5 as described herein may release about 675 μg/cm² of the drug in 5 hours. In embodiments, a composition at pH 6.5 as described herein may release about 600 μg/cm², about 625 μg/cm², about 650 μg/cm², or about 700 μg/cm² of the drug in 5 hours.

In embodiments, a composition at pH 3.5 as described herein may release the drug at about 260 μg/hr/cm². In embodiments, a composition at pH 3.5 as described herein may release the drug at about 200 μg/hr/cm², about 220 μg/hr/cm², about 240 μg/hr/cm², about 260 μg/hr/cm², about 280 μg/hr/cm², or about 300 μg/hr/cm².

In embodiments, a composition at pH 4.5 as described herein may release the drug at about 305 μg/hr/cm². In embodiments, a composition at pH 4.5 as described herein may release the drug at about 275 μg/hr/cm², about 300 μg/hr/cm², about 320 μg/hr/cm², about 340 μg/hr/cm², about 360 μg/hr/cm², or about 400 μg/hr/cm².

In embodiments, a composition at pH 5.5 as described herein may release the drug at about 340 μg/hr/cm². In embodiments, a composition at pH 5.5 as described herein may release the drug at about 300 μg/hr/cm², about 320 μg/hr/cm², about 340 μg/hr/cm², about 360 μg/hr/cm², about 400 μg/hr/cm², or about 430 μg/hr/cm².

In embodiments, a composition at pH 6.5 as described herein may release the drug at about 365 μg/hr/cm². In embodiments, a composition at pH 6.5 as described herein may release the drug at about 300 μg/hr/cm², about 320 μg/hr/cm², about 340 μg/hr/cm², about 360 μg/hr/cm², about 400 μg/hr/cm², or about 450 μg/hr/cm².

In embodiments, the average release rate of a composition as described herein may have a positive correlation with the pH of the composition. In embodiments, a composition at pH 3.5 as described herein may release the drug at between about 25 μg/cm² and about 280 g/cm². In embodiments, a composition at pH 4.5 as described herein may release the drug at between about 60 g/cm² and about 440 μg/cm². In embodiments, a composition at pH 5.5 as described herein may release the drug at between about 115 μg/cm² and about 510 μg/cm². In embodiments, a composition at pH 6.5 as described herein may release the drug at between about 225 μg/cm² and about 680 μg/cm².

In embodiments, the average release rate of an active ingredient in a composition as described herein may have a negative correlation with the particle size of the active ingredient. In embodiments, particles of an active ingredient in a composition as described herein may be a D10 particle size. In embodiments, particles of an active ingredient in a composition as described herein may be a D50 particle size. In embodiments, particles of an active ingredient in a composition as described herein may be a D90 particle size. In embodiments, the particles comprise an average particle size, e.g., D50 particle size, of about 0.5 microns (0.5 μm) to about 20 microns (20 μm). In embodiments, the particles comprise an average particle size, e.g., D50 particle size, of about 1 micron (1 μm) to about 17 microns (17 μm). In embodiments, the particles comprise an average particle size, e.g., D50 particle size, of about 2 microns (2 μm) to about 20 microns (20 μm). In embodiments, the particles comprise an average particle size, e.g., D50 particle size, of about 1 microns (1 μm) to about 10 microns (10 μm). In embodiments, the particles comprise an average particle size, e.g., D50 particle size, of about 1 micron (1 μm). In embodiments, the particles comprise an average particle size of about 1.5 microns (1.5 μm). In embodiments, the particles comprise an average particle size of about 2 microns (2 μm). In embodiments, the particles comprise an average particle size of about 2.5 microns (2.5 μm). In embodiments, the particles herein comprise an average particle size of about 3 microns (3 μm). In embodiments, the particles comprise an average particle size of about 3.5 microns (3.5 μm). In embodiments, the particles comprise an average particle size of about 4 microns (4 μm). In some embodiments, the active ingredient is micronized.

In addition, in one set of embodiments, the composition may have a relatively low concentration or amount of air. For instance, in one embodiment, the composition is substantially free of air. In some cases, during manufacture, a large amount of air may be introduced into the composition, e.g., as foam or bubbles, etc. However, this air may be undesirable in accordance with certain embodiments, and accordingly, the composition may be prepared by also including a step of removing air from the composition that has been introduced during manufacture.

In some embodiments, the composition, after removing at least some of the air may contain no more than 20 vol %, no more than 15 vol %, no more than 12 vol %, no more than 10 vol %, no more than 8 vol %, no more than 6 vol %, no more than 5 vol %, no more than 4 vol %, no more than 3 vol %, no more than 2 vol %, or no more than 1 vol %, etc. of air. In some cases, the air may be removed such that no air bubbles are visually present within the composition.

Without wishing to be bound by any theory, it is believed that the presence of air may reduce the viscosity of the composition, e.g., making it easier for the composition to flow. Accordingly, in some embodiments, any air that is introduced may be removed, thereby increasing the viscosity of the composition, e.g., to at least 800,000 cP, or other ranges of viscosities, such as any of those described herein. Furthermore, in some cases, removing the air may also increase the density of the final composition. For instance, the density of the composition may be at least 0.98 g/cm³, at least 0.99 g/cm³, at least 1 g/cm³, at least 1.01 g/cm³, at least 1.02 g/cm³, at least 1.03 g/cm³, at least 1.05 g/cm³, at least 1.1 g/cm³, etc. In some cases, the density of the composition may be no more than 1.1 g/cm³, no more than 1.05 g/cm³, no more than 1.03 g/cm³, no more than 1.02 g/cm³, no more than 1.01 g/cm³, etc. Furthermore, in some embodiments, combinations of any of these ranges are possible. For example, the final density of the composition may be between 1.00 g/cm³ and 1.01 g/cm³, between 0.99 g/cm³ and 1.02 g/cm³, etc. In embodiments, the density of the composition may be about 0.976 g/cm³. In embodiments, the density of the composition may be about 0.993 g/cm³. In embodiments, the density of the composition may be about 1.00 g/cm³, 1.01 g/cm³, or 1.02 g/cm³.

In one embodiment, air may be removed from a composition, e.g., during or after formation, by applying a pressure less than atmospheric or ambient pressure to the composition. For instance, the pressure that is applied may be less than 1 bar, less than 800 mbar, less than 600 mbar, less than 500 mbar, less than 400 mbar, less than 300 mbar, less than 200 mbar, less than 100 mbar, less than 75 mbar, less than 60 mbar, less than 50 mbar, less than 40 mbar, less than 30 mbar, less than 20 mbar, less than 10 mbar, less than 5 mbar, less than 3 mbar, less than 2 mbar, less than 1 mbar, etc. It should be noted that 1 atmosphere is approximately 1 bar, and that these pressures are absolute pressures (i.e., a pressure of less than about 1 bar means a pressure lower than atmospheric pressure, i.e., a vacuum pressure). Such pressures may be applied for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc. In addition, in some cases, the pressure may be applied until the composition comprises less than a certain amount of air, e.g., less than 15 vol %, or other percentages such as those described herein. As yet another example, in some cases, a solution may be caused to form a gel by removing a certain amount of air from the solution.

In certain embodiments, as another example, the air may be removed from the composition using a Versator or other deaerator, degasser, and/or defoamer. In a Versator, a material such as a liquid is spread onto the inside of a rotating Versator disc under vacuum to remove entrapped air, foam, gas, etc. While the liquid travels across the disc, the high vacuum draws off the bubbles, etc., from the liquid. Versators may be obtained from several commercial sources. Accordingly, in some embodiments, a composition such as described herein may be treated using a Versator for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc., and/or until the composition comprises less than a certain amount of air, e.g., less than 15 vol %, or other percentages such as those described herein

As yet another example, in some cases, the air may be removed from the composition using centrifugation. Without wishing to be bound by any theory, it is believed that by centrifuging the composition, air (being less dense) may be forced out of the composition. Thus, for example, the material may be centrifuged at any suitable speed, e.g., at least at least 500 RPM, at least 1,000 RPM, at least 2,000 RPM, at least 3,000 RPM, at least 5,000, at least 10,000 RPM, or the like, for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc., and/or until the composition comprises less than a certain amount of air, e.g., less than 15 vol %, or other percentages such as those described herein.

Furthermore, it should be understood that these techniques are non-limiting, and that other methods of removing air, besides centrifuges or Versators, are also possible in still other embodiments

In embodiments, the product can be manually or filled using an automated filling machine. In some embodiments, the automated filling machine (e.g., a Capmatic automated filing machine) is especially designed to fill applicator-based products, such as vaginal applicator-based products. As the viscosity of the product decreased at refrigerated temperatures, e.g., at temperatures of about 4° C. (e.g., a reduction in viscosity of 2-3×), the product may become suitable for automated filling into a vaginal applicator. In some embodiments, a cooled, jacketed vessel, feeding cooled product into the filler hopper to fill the applicators is used.

### Methods of Use

Accordingly, as discussed above, a variety of compositions are covered in various embodiments, including any suitable combinations of any of the above-described components, such as poloxamer, xanthan gum and/or another stabilization polymer, an active ingredient, and water. For example, in one set of embodiments, at least 50 wt %, at least 60 wt %, at least 70 wt %, at least 80 wt %, at least 90 wt %, at least 95 wt %, or at least 99 wt % of the composition comprises or consists essentially of polymer such as a poloxamer, xanthan gum and/or another stabilization polymer, an active ingredient, and water. The active ingredient may include any of those described herein, for example diclofenac, etc., as well as salts thereof.

In some embodiments, between about 1 ml and about 10 ml of a composition described herein is administered to a subject. In some embodiments, between about 1 ml and about 6 ml of a composition described herein is administered to a subject. In some embodiments, about 1 ml of a composition described herein is administered to a subject. In some embodiments, about 1.5 ml of a composition described herein is administered to a subject. In some embodiments, about 2 ml of a composition described herein is administered to a subject. In some embodiments, about 2.5 ml of a composition described herein is administered to a subject. In some embodiments, about 3 ml of a composition described herein is administered to a subject. In some embodiments, about 3.5 ml of a composition described herein is administered to a subject. In some embodiments, about 4 ml of a composition described herein is administered to a subject. In some embodiments, about 4.5 ml of a composition described herein is administered to a subject. In some embodiments, about 5 ml of a composition described herein is administered to a subject. In some embodiments, about 5.5 ml of a composition described herein is administered to a subject. In some embodiments, about 6 ml of a composition described herein is administered to a subject.

Furthermore, as mentioned, certain aspects as described herein are generally directed to compositions and methods for applying such compositions for the treatment or prevention of indications such as any of those described herein, e.g., to the vagina of a subject, such as a human. The subject can also be a non-human animal. In embodiments, the subject is female.

In another set of embodiments, a composition such is described herein may be used to treat a subject having or at risk of dysmenorrhea or menstrual cramps. These are often caused by uterine contractions during menstruation. Although some menstrual pain is normal, subjects with dysmenorrhea often experience pain so severe that it prevents them from doing normal activities for several days, often lasting 3, 4, or more days. In some cases, a subject may be treated by applying a composition such as is described herein to the vagina to treat the dysmenorrhea. The composition may have a suitable active ingredient, such as diclofenac and/or a salt thereof, and/or other active ingredients such as those described herein. After insertion, the active ingredient may be released, e.g., into the vagina or cervix to treat or prevent dysmenorrhea.

In another set of embodiments, a composition such as described herein may be used to treat a subject having or at risk of having menorrhagia. Menorrhagia is heavy menstrual bleeding. It is a common disorder in adolescents and reproductive age women and if untreated, can result in anemia, work and school absenteeism and other disruptions to activities of daily living. Dysmenorrhea and menorrhagia are also common side effects of the copper (Cu) intrauterine device (IUD). First line, non-hormonal treatments of menorrhagia include administration of oral non-steroidal anti-inflammatory drugs (NSAIDs), such as diclofenac. Data support that oral NSAIDs reduce menorrhagia by similar mechanisms which also result in reduced dysmenorrhea, including reduction in endometrial leukotrienes and inflammation. Tranexamic acid is also a non-hormonal medication which significantly reduces menorrhagia, however, this non-hormonal agent cannot be used long term due to the risk of thrombosis.

The advantage of oral NSAIDs is that they are generally available over-the-counter and are effective in reducing menorrhagia and dysmenorrhea, even in Cu IUD users. However, common dose-dependent side effects of oral NSAIDs include gastrointestinal bleeding, diarrhea, and nausea. In addition, taking daily oral NSAIDs for several months to manage menorrhagia and or dysmenorrhea and or side effects of Cu IUDs requires high adherence, especially difficult in younger populations and most importantly long term systemic use of NSAIDs can result in renal damage. Oral NSAIDs are also a first line non-hormonal treatment for the management of generalized pelvic pain, acute pelvic pain and chronic pelvic pain, which can result from endometriosis, adenomyosis, ovulatory pain (Mittleschmirtz), ovarian cyst pain and/or pelvic muscle associated pain.

The oral NSAID dose and the frequency of oral dosing are the same for the treatment of menorrhagia, dysmenorrhea, generalized pelvic pain, acute pelvic pain, ovulatory pain, ovarian cyst pain and pelvic muscle associated pain. Oral NSAIDs are most commonly given as needed or “prn” for the treatment of pain. The duration of treatment for oral NSAIDs is generally shorter for acute pelvic pain, ovulatory pain, dysmenorrhea and menorrhagia. Longer, chronic administration of NSAIDs is generally required for chronic conditions such as endometriosis, adenomyosis, and generalized pelvic pain. It is expected that the treatment duration for the diclofenac vaginal hydrogel would follow the same flexible dosing schedule, based on the individual's specific pain levels and causative factors.

### Local Genital Delivery of NSAIDS for Dysmenorrhea and Menorrhagia

Local delivery of active pharmaceutical ingredients through the vaginal mucosa is ideal because lower doses can be used to target local genital tissues and the myometrium. Data consistently support that drugs delivered vaginally concentrate 10-100 times higher in genital tissues and fluids compared to systemic blood concentrations, which reduces systemic side effects. This is particularly important when the expectation is that a patient will need longer term or chronic NSAID therapy for conditions such as endometriosis or generalized pelvic pain.

In another set of embodiments, a composition such as described herein may be used to treat a subject having or at risk of having generalized pelvic pain, acute pelvic pain and chronic pelvic pain. Without wishing to be bound by theory, these can result from endometriosis, adenomyosis, ovulatory pain, ovarian cyst pain and or pelvic muscle associated pain.

Dosing may or may not be different depending on the underlying cause. In some embodiments, the only difference in dosing is the duration of treatment, e.g., where chronic pelvic pain from chronic conditions (such as endometriosis) likely needs longer durations of treatment (e.g. weeks to months) whereas acute pain or pain flares (e.g. dysmenorrhea, ovulatory pain) likely requires a shorter duration of pain. Most of the time oral NSAIDs are dosed as needed, for example, for dysmenorrhea this is normally during the premenstrual time, when uterine contractions are beginning and for maybe the first few days of menses.

In embodiments, the composition is administered to a subject between one and ten times. In embodiments, the composition is administered to a subject between one and ten times per menstrual cycle. In embodiments, the composition is administered to a subject between one and five times per menstrual cycle. In embodiments, the composition is administered to a subject between one and three times per menstrual cycle. In embodiments, the composition is administered to a subject one time per menstrual cycle. In embodiments, the composition is administered to a subject twice per menstrual cycle.

In embodiments, the composition is administered daily. In embodiments, the composition is administered every two days. In embodiments, the composition is administered every 3, 4, 5, 6, 7, 8, 9, 10, 20, 28, or 30 days. In embodiments, the composition is administered as needed, for example when the subject is in pain.

Terms such as “treat,” “treatment,” “treating,” etc. comprise therapeutic treatment of subjects having already developed a disease, in particular in manifest form. Therapeutic treatment may be symptomatic treatment in order to relieve the signs and/or symptoms of the disease or causal treatment in order to reverse, partially reverse, stop, or slow down the progression of the disease. Thus, the compositions and methods of the present disclosure may be used, for instance, as therapeutic treatment (e.g., for acute or chronic therapy).

Additionally, terms such as “prevent,” “preventing,” or “prevention” generally refer to the reduction of the occurrence of the disease, and/or a sign and/or symptom thereof, in the treated sample relative to an untreated control sample, or delays the onset of one or more signs and/or symptoms of the disease relative to the untreated control sample, in a statistically significant manner. Preventing the disease, and/or a sign and/or a symptom thereof, includes preventing or delaying the initiation of the disease, sign, and/or symptom. Prevention also includes preventing a recurrence of the disease, sign, and/or symptom.

In certain aspects, the composition can be applied to a subject, e.g., to the vagina of a subject, and/or to another body cavity, for example, the mouth or the rectum. Any suitable technique may be used to apply the composition to the subject. For instance, the composition may be free or mass flowing, e.g., so that it may be administered through an applicator or other suitable device. Thus, in some embodiments, the composition may be contained within applicator, such as a vaginal applicator or a syringe, which can be applied, e.g., by the subject, or by another person.

The subject may be, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., monkeys such as cynomolgus monkeys or rhesus monkeys, chimpanzees, etc.); commercially relevant mammals such as cattle, pigs, horses, sheep, rabbits, mice, rats, goats, cats, dogs, etc.) and birds (e.g., commercially relevant birds such as chickens, ducks, geese, turkeys, etc.). In certain embodiments, the subject is a mammal. The subject may be a male or female and at any stage of development. A non-human animal may be a transgenic animal.

In one set of embodiments, as discussed, the composition is applied to treat the subject with a therapeutically effective amount of an active ingredient, such as any of those described herein. The therapeutically effective amount may be an amount which, when administered to a subject for treating or preventing a disease, is sufficient to effect such treatment or prevention for the disease, for example, any of those described herein. The therapeutically effective amount may also be an amount sufficient to elicit a desired biological response, i.e., alleviating a symptom. The therapeutically effective amount may vary depending on such factors as the desired biological endpoint, the mode of administration, and/or the age and health of the subject.

### Kits

Still another aspect of the present disclosure is directed to kits. The kit may include a package or an assembly including one or more of the compositions as described herein, and/or other compositions. Each of the compositions of the kit may be provided in liquid form (e.g., in solution), or in solid form (e.g., a dried powder), or in gaseous form in some cases. In certain cases, some of the compositions may be constitutable or otherwise processable (e.g., to an active form), for example, by the addition of a suitable solvent or other species, which may or may not be provided with the kit. Examples of other components include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

A kit may, in some cases, include instructions in any form that are provided in connection with the compositions described herein in such a manner that one of ordinary skill in the art would recognize that the instructions are to be associated with those compositions. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In an embodiment, provided herein are kits comprising the compositions and gels described herein. For example, the kit includes an applicator suitable for vaginal application. In other examples, the applicator is pre-filled with the compositions or the gels described herein. In other embodiments, the applicator is not-prefilled with the compositions or the gels described herein. In some embodiments, the compositions or gels described herein are at refrigerated temperatures (e.g., about 4° C.) prior to pre-filling the applicator. In some embodiments, the kit includes an applicator that is pre-filled with the composition, where the composition includes a poloxamer, a stabilization polymer, and an active ingredient (e.g., diclofenac or any other therapeutic agent for treating dysmenorrhea or treating primary dysmenorrhea).

In some embodiments, the kit further includes one or more instructions for inserting the applicator into the vagina. In other embodiments, the kit includes instructions for treating dysmenorrhea or treating primary dismenorrhea by applying the applicator filled with the compositions or gels described herein. In some embodiments, the kit further includes instructions for filling the applicator with the composition or gel.

In some embodiments, the compositions described herein are stored at room temperature (either pre-filled in an applicator or a separate storage container). In some embodiments, the compositions can be stored for 1 day, 2 days, 10 days, 2 weeks, 1 month, 2 months, 3 months, 4 months, 6 months, 7 months, 8 months, 9 months, 10 months, 11 months, 1 year, or more at room temperature.

### EXAMPLES

The following examples are intended to illustrate certain embodiments of the present disclosure, but do not exemplify the full scope of the disclosure.

### Example 1: Composition

In this prophetic example, a composition in accordance with one embodiment is as follows (all components USP grade). The active ingredient may be diclofenac, or the like.

TABLE 3
composition

| Material | g/100 g |
|---|---|
| Poloxamer 407 | 16.50 |
| Xanthan gum | 2.00 |
| Active ingredient | 2.00 |
| Citric acid monohydrate | 0.60 |
| Sodium citrate dihydrate | 0.55 |
| Benzyl alcohol | 1.00 |
| Purified water (qs) | 77.35 |
| Total | 100 |

### Example 2: Method of Making the Composition

This prophetic example illustrates a method of making the composition described in Example 1, in accordance with another embodiment.

Purified water is added to a kettle (or other suitable container), which is placed under a dissolver (30-60 HP) with a 12-inch stand dissolver blade. The dissolver is started, and an active ingredient is added to the kettle. The dissolver is allowed to mix for at least 10 minutes, or until the active ingredient has visually dissolved in the water.

Afterwards, mixing is continued while citric acid monohydrate and sodium citrate dihydrate are added to the kettle. The kettle is then cooled with chilled cooling water, e.g., a temperature of 8° C. Mixing is continued for at least 5 minutes, or until these have visually dissolved.

Mixing and cooling then continues while benzyl alcohol and poloxamer 407 is added. These are mixed until the water is clear and everything has visually dissolved.

Mixing and cooling continues while xanthan gum is slowly added. Mixing of the xanthan gum continues for at least 10 minutes, or until the composition is visually uniform. The speed of mixing may be adjusted, for example, as the composition thickens and its viscosity increases, and/or to avoid trapping too many air bubbles within the composition.

Next, the composition is transferred into a round-bottom, jacketed, stainless steel pressure/vacuum kettle, or another suitable container. The kettle is cooled as before, e.g., using chilled cooling water at 8° C. Mixing in the kettle is started and the batch is slowly recirculated under a vacuum. Mixing, cooling with chilled cooling water, and recirculating under vacuum occurs for at least 30 minutes, or until the composition has been sufficiently deaerated.

It should be understood that the above is by way of example only, and that other techniques for making the compositions described herein are also contemplated. For example, the addition sequence of poloxamer 407 and xanthan gum may be reversed, with the poloxamer going into the mixing first. Air may be removed using a centrifuge, a Versator, or the like.

### Example 3: Method of Making the Composition

This prophetic example illustrates a method of making a composition in accordance with another embodiment.

The primary compounding phase in this example is prepared by adding 154 kg of Purified Water USP to a 270 L kettle. The kettle is placed under a dissolver equipped with a 10-inch standard blade. Mixing commences at about 600 rpm. 4.86 kg of an active ingredient is added and mixed for 10 minutes until visually dissolved. Mixing continues and 1200 g citric acid and 1100 g of sodium citrate are added and mixed for 6 min until visually dissolved. Cooling of the batch is started during this step by flowing chilled water through the kettle jacket.

Mixing continues and 2000 g of benzyl alcohol and 33.0 kg of poloxamer 407 are added. Cooling and mixing continue until the solution is clear and the poloxamer is visually dissolved. The batch temperature reaches approximately 11° C. during this step. This step creates foam on the surface of the batch and the mixing speed is decreased to allow the foam to dissipate. Mixing speed is then increased, eventually reaching 1200 rpm for addition of the 4.00 kg of xanthan gum. After the addition of the xanthan gum, the product rapidly increases in viscosity and the original mixing blade size is switched to a 12-inch blade to better facilitate mixing at this stage of the process. A 14-inch blade may also be used at this scale.

After this step in the process, the product has generated a large amount of foaming. The foaming can be reduced by transferring the product into a cooling, jacketed vacuum vessel and recirculate the product for several passes to help remove the air. Another approach is to use a Versator, which is a device for removing air from liquids and semi-solids. The product was passed through the Versator several times until all air was removed. Measurements of the viscosity after Versator treatment rose to over 3 million cP, from 1.2 million cP at room temperature prior to using the Versator.

### Example 4: Viscosity of the Composition

The present example shows the viscosity of a composition in accordance with one embodiment as a function of temperature. It will be noted that, even at room temperature (about 25° C.), the composition has a viscosity of over 1 million cP.

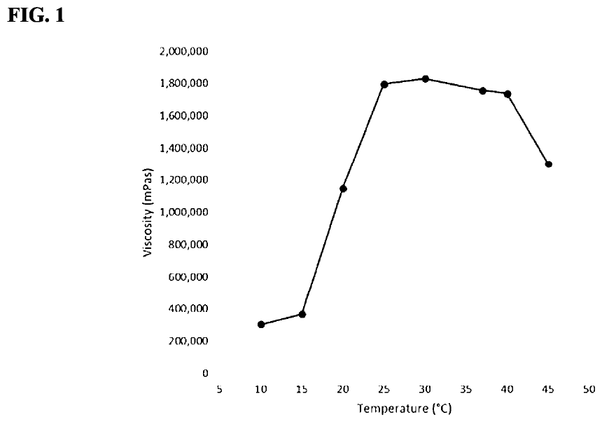

FIG. 1 illustrates the viscosity (in mPA*s, equilavent to cP) plotted as a function of temperature for the composition shown in Example 1. Viscosity was measured using a Brookfield Viscometer.

In this experiment, the viscosity at 25° C. was found to be 1,798,000 cP, while the viscosity at 37° C. (body temperature) was found to be 1,759,000 cP.

### Example 5: In Vitro Release Testing (IVRT) Results

In vitro release testing (IVRT) was evaluated for diclofenac sodium gel 1%, 2%, and 3% at n=6 using a PION Rainbow® in situ fiber optic probes with PDA (200-720 nm) with 2 mm stainless steel probes. The results for the average release and release rate (slope) are presented in Table 4 (below).

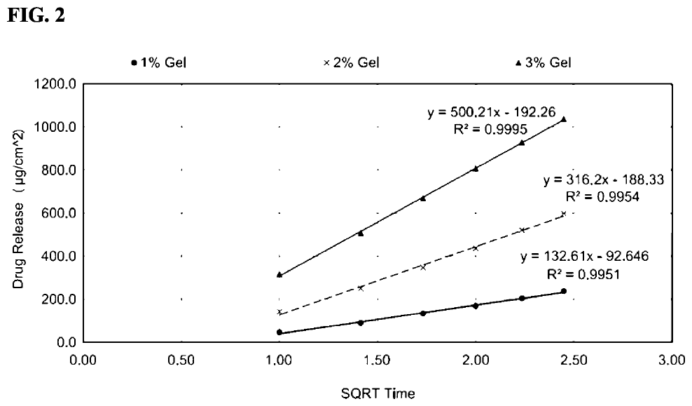

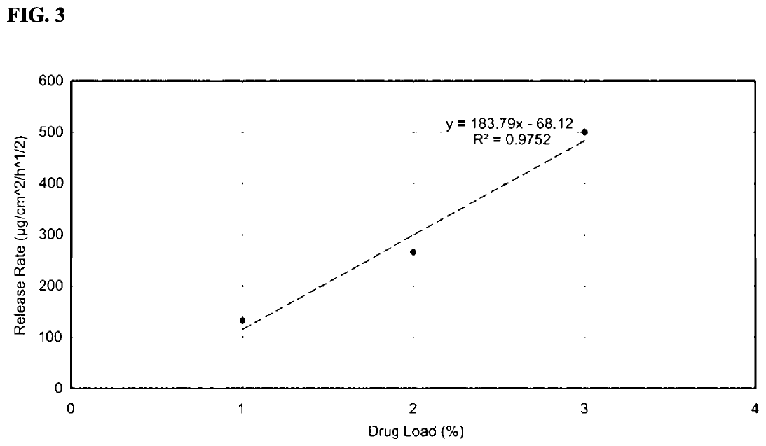

The release rate increased proportionally with the dose (FIG. 2 and FIG. 3). The release rate did not strictly follow the classical Higuchi model for drug release of suspensions from the matrix, which may be explained by the additional changes in the rheology which correlate to the increase solids content in the formulation. The result describes a more complex relationship between the formulation composition and the drug release.

TABLE 4
IVRT Results

| Time | SQRT | Average Drug Release (μg/cm²) | | |
|---|---|---|---|---|
| (hours) | Time | 1% Gel | 2% Gel | 3% Gel |
| 1 | 1.00 | 46.4 | 142.5 | 316.6 |
| 2 | 1.41 | 89.9 | 250.0 | 507.5 |
| 3 | 1.73 | 133.6 | 346.6 | 668.4 |
| 4 | 2.00 | 168.9 | 436.7 | 806.6 |
| 5 | 2.24 | 203.9 | 521.1 | 928.3 |
| 6 | 2.45 | 237.9 | 598.3 | 1037.5 |

| | | | |
|---|---|---|---|
| Slope (Rate) | 132.6 | 316.2 | 500.2 |
| R² | 0.995 | 0.995 | 0.999 |
| % RSD | 2.2 | 4.3 | 2.5 |

The release rates obtained during IVRT for the three test batches were used to calculate the estimated time for 50, 80, and 100% drug release (Table 5, below) based on a dose coverage of ~0.23 g/cm² assuming a constant release rate. Typically, the release rate slowed as the dose is depleted so it would be expected that the actual times for 80% and 100% drug release would be longer than provided by this estimate.

There are at least two notable aspects of this release data. First, diclofenac is in suspension, not solution, within the gel. As compared to poloxamer gel containing a solubilized active agent (e.g., clindamycin), diclofenac would be expected to take longer to release out of an equivalent amount of gel matrix. Second, a relationship between pH and diclofenac release rate has been observed. It is implicit in the data in Table 4 showing a non-linear variance in release rate as the API concentration goes up. When combined with the data in Table 17 that the pH goes up as the API concentration goes up, it supports the conclusion that higher pH supports faster release. Table 5 further supports this by showing that the 3% gel releases 50%, 80% and 100% faster than 1% or 2% diclofenac compositions.

TABLE 5
Estimated time for diclofenac release

| | Time (hours) | | |
|---|---|---|---|
| | 50% Drug | 80% Drug | 100% Drug |
| Sample | Release | Release | Release |
| 1% Gel | 30 | 47 | 59 |
| 2% Gel | 25 | 40 | 50 |
| 3% Gel | 24 | 40 | 47 |

### IVRT for Diclofenac Gel at 1% and 3% w/w

Additional IVRT was performed for diclofenac vaginal gel 1% and 3% w/w. For this analysis the receptor media was changed from phosphate buffer pH 7 to simulated vaginal fluid. Sampling was executed every 60 minutes for 6 hours, and a single point at T=24 hours. Total volume replacement at each time point was implemented due the low solubility of the drug substance in the simulated vaginal fluid (~16 μg/mL).

The results for both dose strengths followed the square root (t) model through 24 hours. The drug release was linear with time through at least 6 hours, and then began to taper off slightly by 24 hours. This demonstrated a typical release profile for a drug substance suspended in a viscoelastic matrix. The vessel-to-vessel variance (% RSD) was <4% for both runs, and the R² values for the SQRT (t) was >0.99 through 24 hours.

From the data collected through 24 hours the drug release versus time for the applied dose (~0.4 g) was estimated according to the Higuchi square root equation based on the total surface area of the IVRT membrane, and the average surface area of the vagina (see, P. Pendergrass, M Belovicz. Surface Area of the Human Vagina as Measured from Vinyl Polysiloxane Casts. February 2003. Gynecologic and Obstetric Investigation 55 (2): 110-3, incorporated herein by reference in its entirety). The percent drug release at T=24 hours (4.3% and 2.4% respectively) was included in the estimate for comparison to the measured result to the regression model. Tables 6 and 7 below show the estimated relates for the 1% diclofenac gel and 3% diclofenac gel, respectively.

TABLE 6
Estimated Release for the 1% Diclofenac Vaginal Gel

| | | | Est. In |
|---|---|---|---|
| | Total μg | IVRT hours | Vivo hours |
| % Release | Release | (1.77 cm\hat{ }2) | (87.5 cm\hat{ }2) |
| 100 | 4000 | 10272.3 | 4.2 |
| 80 | 3200 | 6574.2 | 2.7 |
| 50 | 2000 | 2568.1 | 1.1 |
| 25 | 1000 | 642.0 | 0.3 |
| 4.3 | 174 | 19.4 | 0.0 |

TABLE 7
Estimated Release for the 3% Diclofenac Vaginal Gel

| | | | Est. In |
|---|---|---|---|
| | Total μg | IVRT hours | Vivo hours |
| % Release | Release | (1.77 cm\hat{ }2) | (87.5 cm\hat{ }2) |
| 100 | 12000 | 46358.5 | 19.0 |
| 80 | 9600 | 29669.5 | 12.1 |
| 50 | 6000 | 11589.6 | 4.7 |
| 25 | 3000 | 2897.4 | 1.2 |
| 2.4 | 287 | 26.5 | 0.01 |

The estimated time assumes the parameters of the Higuchi model are maintained throughout the drug release and do not account for total dose depletion, pH changes, swelling of the matrix, or dissolution of the gel. As an estimate the relationship between the total dose and drug release time can be modeled as the change in dose squared:

$$R T 2 = {( \frac{Q 2}{Q 1} )}^{2} * R T 1$$

### Where:

RT is the release time
Q1 is the reference dose
Q2 is the new dose

Example: Change in 100% release time of a 1% gel for a 0.4 g dose to a 1 g dose based on a surface area of 87.5 cm².

$$R T = {( \frac{1.0 * 10000}{0.4 * 10000} )}^{2} * 4.2 hours = 26.3 hours$$

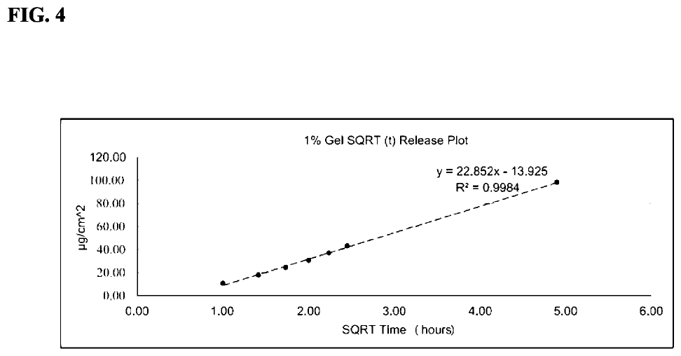

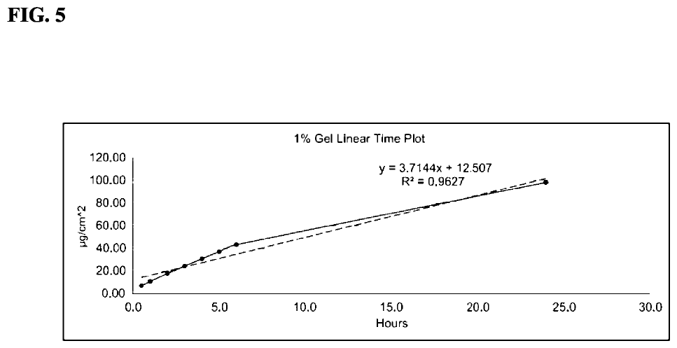

TABLE 8
IVRT Results for 1% w/w Diclofenac Gel (see also FIG. 4 and FIG. 5)

| | sqrt | μg/cm² | | | | | | Avg. |
|---|---|---|---|---|---|---|---|---|
| Hours | (hrs) | 1 | 2 | 3 | 4 | 5 | 6 | μg/cm² |
| 0.5 | 0.71 | 7.13 | 6.96 | 8.85 | 6.53 | 5.53 | 7.34 | 7.06 |
| 1.0 | 1.00 | 10.61 | 10.51 | 13.11 | 10.59 | 8.45 | 11.21 | 10.75 |
| 2.0 | 1.41 | 17.17 | 17.26 | 20.14 | 18.20 | 15.19 | 18.49 | 17.74 |
| 3.0 | 1.73 | 23.39 | 23.51 | 26.76 | 25.33 | 21.61 | 25.23 | 24.30 |
| 4.0 | 2.00 | 29.43 | 29.54 | 33.54 | 32.28 | 27.80 | 31.68 | 30.71 |
| 5.0 | 2.24 | 35.50 | 35.50 | 40.22 | 39.21 | 33.79 | 38.01 | 37.04 |
| 6.0 | 2.45 | 41.78 | 41.37 | 46.71 | 45.84 | 39.58 | 44.12 | 43.23 |
| 24.0 | 4.90 | 96.08 | 93.23 | 100.52 | 103.37 | 95.95 | 100.18 | 98.22 |

| | | | | | | | | |
|---|---|---|---|---|---|---|---|---|
| Linear Slope | 3.6 | 3.5 | 3.7 | 3.9 | 3.7 | 3.8 | 3.71 | |
| R² | 0.967 | 0.962 | 0.956 | 0.958 | 0.970 | 0.962 | Avg. Slop | 22.3 |
| | | | | | | | (SQRT (t) | |
| SQRT (t) Slope | 21.8 | 21.1 | 22.4 | 23.7 | 22.2 | 22.7 | Linear Slope | 3.8 |
| | | | | | | | % RSD | |
| R² | 0.995 | 0.996 | 0.996 | 0.996 | 0.994 | 0.996 | SQRT(t) Slope | 3.9 |
| | | | | | | | % RSD | |

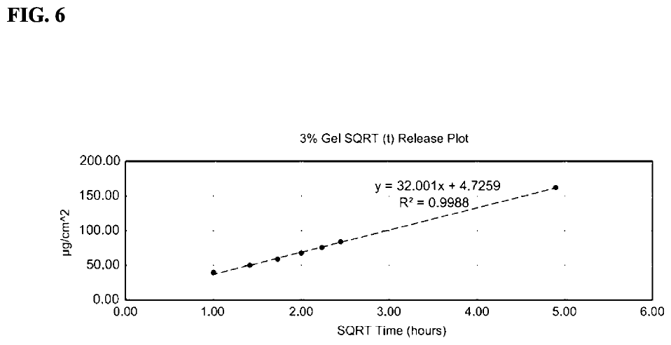

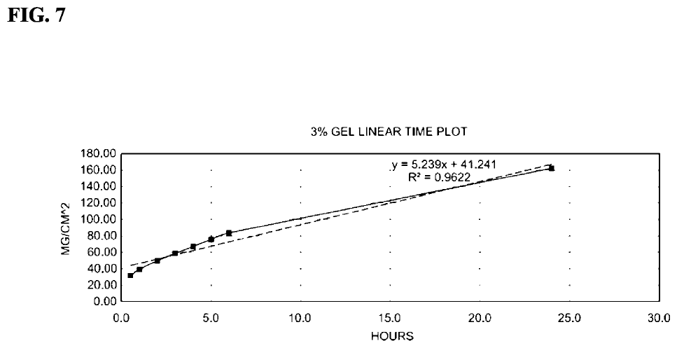

TABLE 9
IVRT Results for 3% w/w Diclofenac Gel (see also FIGS. 6 and 7)

| | sqrt | μg/cm\hat{ }2 | | | | | | |
|---|---|---|---|---|---|---|---|---|
| Hours | (hrs) | 1 | 2 | 3 | 4 | 5 | 6 | Avg. |
| 0.5 | 0.71 | 32.25 | 32.24 | 25.21 | 24.28 | 45.12 | 47.44 | 31.82 |
| 1.0 | 1.00 | 40.23 | 39.11 | 32.87 | 31.08 | 53.23 | 92.29 | 39.30 |
| 2.0 | 1.41 | 51.22 | 48.89 | 43.60 | 41.66 | 63.28 | 129.12 | 49.73 |
| 3.0 | 1.73 | 60.10 | 57.60 | 52.78 | 51.37 | 71.94 | 195.33 | 58.76 |
| 4.0 | 2.00 | 68.68 | 65.73 | 61.55 | 59.95 | 80.32 | 265.81 | 67.25 |
| 5.0 | 2.24 | 76.73 | 73.43 | 69.78 | 68.91 | 89.05 | 334.58 | 75.58 |
| 6.0 | 2.45 | 84.76 | 81.15 | 77.64 | 77.22 | 97.69 | 407.06 | 83.69 |
| 24.0 | 4.90 | 161.91 | 154.95 | 159.56 | 162.58 | 171.88 | 532.51 | 162.18 |

| | | | | | | | | |
|---|---|---|---|---|---|---|---|---|
| Linear Slope | 5.2 | 4.9 | 5.4 | 5.6 | 5.1 | 18.4 | 5.2 | |
| R² | 0.958 | 0.962 | 0.965 | 0.969 | 0.956 | 0.709 | Avg. | 31.5 |
| | | | | | | | SQRT(t) | |
| | | | | | | | Slope | |
| SQRT (t) Slope | 31.2 | 29.7 | 32.4 | 33.5 | 30.6 | 120.6 | Linear Slope | 5.1 |
| | | | | | | | % RSD | |
| R² | 0.999 | 0.998 | 0.997 | 0.996 | 0.998 | 0.873 | SQRT(t) Slope | 4.9 |
| | | | | | | | % RSD | |

### Example 6: Release Rate Evaluation

This study evaluated the release rate of diclofenac by in-vitro release testing of diclofenac 1% and 3% gel in simulated vaginal fluid. Two liters of simulated vaginal fluid was prepared and the release rate of diclofenac from 1% and 3% gels was evaluated. The formulation composition for simulated vaginal fluid is shown in Table 10 below. To prepare the simulated vaginal fluid, ~1700 mL of purified water was transferred into a tared 2 L beaker with a stir bar, the components were mixed until dissolved, the pH was adjusted to 4.5 with 1.0N HCl/NaOH, the solution was transfer into a 2 L volumetric flask and diluted volumetrically with purified water and mix well, and the solution was degased for 30 minutes prior to use.

TABLE 10
Formulation Composition of Simulated Vaginal Fluid

| | | | | Amount/ | Amount |
|---|---|---|---|---|---|
| Item | | | | Batch | Weighed |
| No. | Ingredient | % w/w | mg/mL | (g) | (g) |
| 1 | Sodium Chloride | 0.351 | 3.510 | 7.020 | |
| 2 | Potassium | 0.140 | 1.400 | 2.800 | |
| | Hydroxide | | | | |
| 3 | Calcium | 0.022 | 0.222 | 0.444 | |
| | Hydroxide | | | | |
| 4 | Bovine Serum | 0.002 | 0.018 | 0.036 | |
| | Albumin | | | | |
| 5 | Lactic Acid | 0.200 | 2.000 | 4.000 | |
| 6 | Acetic Acid | 0.100 | 1.000 | 2.000 | |
| 7 | Glycerol | 0.016 | 0.160 | 0.320 | |
| 8 | Urea | 0.040 | 0.400 | 0.800 | |
| 9 | D(+)-Glucose | 0.500 | 5.000 | 10.000 | |
| 10 | pH to 4.5 | QS | QS | QS | |
| | (HCl/NaOH) | | | | |
| 11 | Water | 98.629 | 986.29 | 1972.580 | |
| | Total | 100.000 | 1000.000 | 2000.000 | |

The results of the testing are shown in the tables below, where 178003-2201 corresponds to 1% w/w diclofenac gel, and 178003-2203 corresponds to 3% w/w/diclofenac gel.

TABLE 11
Release data for 178003-2201 (1% w/w diclofenac gel)
30 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.63 | 20.0 | 19.521 | 0.00 | 7.13 |
| 2 | 0.62 | 20.0 | 19.353 | 0.00 | 6.96 |
| 3 | 0.78 | 20.0 | 19.666 | 0.00 | 8.85 |
| 4 | 0.58 | 20.0 | 19.427 | 0.00 | 6.53 |
| 5 | 0.49 | 20.0 | 17.948 | 0.00 | 5.53 |
| 6 | 0.65 | 20.0 | 19.407 | 0.00 | 7.34 |

60 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.32 | 20.0 | 19.633 | 12.32 | 10.61 |
| 2 | 0.33 | 20.0 | 19.424 | 11.92 | 10.51 |
| 3 | 0.39 | 20.0 | 19.675 | 15.40 | 13.11 |
| 4 | 0.38 | 20.0 | 19.557 | 11.23 | 10.59 |
| 5 | 0.31 | 20.0 | 19.162 | 8.78 | 8.45 |
| 6 | 0.36 | 20.0 | 19.488 | 12.61 | 11.21 |

120 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.59 | 20.0 | 19.589 | 18.66 | 17.17 |
| 2 | 0.61 | 20.0 | 19.371 | 18.41 | 17.26 |
| 3 | 0.63 | 20.0 | 19.568 | 23.07 | 20.14 |
| 4 | 0.68 | 20.0 | 19.473 | 18.58 | 18.20 |
| 5 | 0.61 | 20.0 | 19.450 | 14.70 | 15.19 |
| 6 | 0.65 | 20.0 | 19.565 | 19.65 | 18.49 |

180 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.56 | 20.0 | 19.476 | 30.16 | 23.39 |
| 2 | 0.57 | 20.0 | 19.412 | 30.17 | 23.51 |
| 3 | 0.60 | 20.0 | 19.533 | 35.38 | 26.76 |
| 4 | 0.65 | 20.0 | 19.466 | 31.86 | 25.33 |
| 5 | 0.59 | 20.0 | 19.685 | 26.54 | 21.61 |
| 6 | 0.61 | 20.0 | 19.501 | 32.45 | 25.23 |

240 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.55 | 20.0 | 19.476 | 41.10 | 29.43 |
| 2 | 0.55 | 20.0 | 19.368 | 41.27 | 29.54 |
| 3 | 0.61 | 20.0 | 19.472 | 47.08 | 33.54 |
| 4 | 0.63 | 20.0 | 19.484 | 44.50 | 32.28 |
| 5 | 0.56 | 20.0 | 19.682 | 38.06 | 27.80 |
| 6 | 0.59 | 20.0 | 19.461 | 44.36 | 31.68 |

300 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.55 | 20.0 | 19.435 | 51.80 | 35.50 |
| 2 | 0.55 | 20.0 | 19.404 | 51.94 | 35.50 |
| 3 | 0.61 | 20.0 | 19.517 | 59.04 | 40.22 |
| 4 | 0.63 | 20.0 | 19.474 | 56.81 | 39.21 |
| 5 | 0.54 | 20.0 | 19.707 | 49.02 | 33.79 |
| 6 | 0.58 | 20.0 | 19.488 | 55.76 | 38.01 |

360 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.57 | 20.0 | 19.300 | 62.52 | 41.78 |
| 2 | 0.54 | 20.0 | 19.435 | 62.52 | 41.37 |
| 3 | 0.59 | 20.0 | 19.566 | 70.90 | 46.71 |
| 4 | 0.60 | 20.0 | 19.523 | 69.08 | 45.84 |
| 5 | 0.52 | 20.0 | 19.714 | 59.64 | 39.58 |
| 6 | 0.56 | 20.0 | 19.524 | 66.99 | 44.12 |

24 hours

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 4.83 | 20.0 | NA | 73.54 | 96.08 |
| 2 | 4.61 | 20.0 | NA | 72.92 | 93.23 |
| 3 | 4.78 | 20.0 | NA | 82.43 | 100.52 |
| 4 | 5.11 | 20.0 | NA | 80.85 | 103.37 |
| 5 | 5.00 | 20.0 | NA | 69.91 | 95.95 |
| 6 | 4.98 | 20.0 | NA | 77.83 | 100.18 |

TABLE 12
Release data for 178003-2203 (3% w/w diclofenac gel)
30 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 2.85 | 20.0 | 19.640 | 0.00 | 32.25 |
| 2 | 2.85 | 20.0 | 19.336 | 0.00 | 32.24 |
| 3 | 2.23 | 20.0 | 19.496 | 0.00 | 25.21 |
| 4 | 2.15 | 20.0 | 19.559 | 0.00 | 24.28 |
| 5 | 3.99 | 20.0 | 19.154 | 0.00 | 45.12 |
| 6 | 4.20 | 20.0 | 19.231 | 0.00 | 47.44 |

60 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.76 | 20.0 | 19.628 | 56.05 | 40.23 |
| 2 | 0.70 | 20.0 | 19.380 | 55.17 | 39.11 |
| 3 | 0.73 | 20.0 | 19.621 | 43.50 | 32.87 |
| 4 | 0.65 | 20.0 | 19.399 | 42.03 | 31.08 |
| 5 | 0.89 | 20.0 | 19.644 | 76.48 | 53.23 |
| 6 | 4.13 | 20.0 | 19.410 | 80.73 | 92.29 |

120 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.99 | 20.0 | 19.556 | 70.93 | 51.22 |
| 2 | 0.89 | 20.0 | 19.548 | 68.79 | 48.89 |
| 3 | 0.96 | 20.0 | 19.566 | 57.90 | 43.60 |
| 4 | 0.96 | 20.0 | 19.130 | 54.62 | 41.66 |
| 5 | 0.91 | 20.0 | 18.984 | 93.91 | 63.28 |
| 6 | 3.38 | 20.0 | 19.475 | 160.91 | 129.12 |

180 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.81 | 20.0 | 19.594 | 90.21 | 60.10 |
| 2 | 0.79 | 20.0 | 19.401 | 86.13 | 57.60 |
| 3 | 0.83 | 20.0 | 19.558 | 76.76 | 52.78 |
| 4 | 0.90 | 20.0 | 19.317 | 72.91 | 51.37 |
| 5 | 0.81 | 20.0 | 19.562 | 111.09 | 71.94 |
| 6 | 5.95 | 20.0 | 19.476 | 226.76 | 195.33 |

240 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.78 | 20.0 | 19.316 | 106.04 | 68.68 |
| 2 | 0.74 | 20.0 | 19.407 | 101.48 | 65.73 |
| 3 | 0.80 | 20.0 | 19.126 | 93.05 | 61.55 |
| 4 | 0.79 | 20.0 | 19.530 | 90.32 | 59.95 |
| 5 | 0.76 | 20.0 | 19.614 | 126.97 | 80.32 |
| 6 | 6.39 | 20.0 | 19.770 | 342.62 | 265.81 |

300 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.74 | 20.0 | 19.519 | 121.03 | 76.73 |
| 2 | 0.70 | 20.0 | 19.464 | 115.89 | 73.43 |
| 3 | 0.76 | 20.0 | 19.136 | 108.26 | 69.78 |
| 4 | 0.81 | 20.0 | 19.544 | 105.74 | 68.91 |
| 5 | 0.79 | 20.0 | 19.817 | 141.88 | 89.05 |
| 6 | 6.16 | 20.0 | 19.651 | 469.01 | 334.58 |

360 Minutes

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 0.73 | 20.0 | 19.542 | 135.46 | 84.76 |
| 2 | 0.70 | 20.0 | 19.392 | 129.60 | 81.15 |
| 3 | 0.73 | 20.0 | 19.634 | 122.86 | 77.64 |
| 4 | 0.75 | 20.0 | 19.395 | 121.59 | 77.22 |
| 5 | 0.77 | 20.0 | 19.646 | 157.47 | 97.69 |
| 6 | 6.52 | 20.0 | 19.358 | 590.06 | 407.06 |

24 hours

| | | Cell | mL of | Total μg | Total |
|---|---|---|---|---|---|
| | Measured | Volume | Sample | Previously | μg/cm\hat{ }2 |
| Cell | μg/mL | (mL) | collected | Removed | Released |
| 1 | 7.23 | 18.9 | NA | 149.69 | 161.91 |
| 2 | 6.94 | 18.9 | NA | 143.21 | 154.95 |
| 3 | 7.88 | 18.4 | NA | 137.15 | 159.56 |
| 4 | 8.31 | 18.2 | NA | 136.22 | 162.58 |
| 5 | 6.93 | 19.0 | NA | 172.64 | 171.88 |
| 6 | 12.07 | 18.8 | NA | 716.31 | 532.51 |

### Example 7: Compatibility Assay of Diclofenac Gel in Gel Formulation 1

The results of analysis for the compatibility of 1%, 2%, and 3% Diclofenac Sodium Gel in Gel Formulation 1 composition are provided herein. The formulation composition of each Diclofenac Sodium Gel is outlined in Tables 13-15 below.

TABLE 13
Composition of 1% Diclofenac Sodium Gel Batch 178003-2201

| | | | | Amount/Batch |
|---|---|---|---|---|
| Item No. | Ingredient | % w/w | mg/g | (g) |
| 1 | Poloxamer F127 | 16.50 | 165.00 | 82.50 |
| 2 | Xanthan Gum, USP | 2.00 | 20.00 | 10.00 |
| 3 | Diclofenac Sodium | 1.00 | 10.00 | 5.00 |
| 4 | Citric Acid | 0.60 | 6.00 | 3.00 |
| | Monohydrate | | | |
| 5 | Sodium Citrate | 0.55 | 5.50 | 2.75 |
| | Dihydrate | | | |
| 6 | Benzyl Alcohol | 1.00 | 10.00 | 5.00 |
| 7 | Purified Water | 74.35 | 743.50 | 371.75 |
| 8 | Purified Water QS | 4.00 | 40.00 | 20.00 |
| 9 | Total | 100.00 | 1000.00 | 500.00 |

TABLE 14
Composition of 2% Diclofenac Sodium Gel Batch 178003-2202

| | | | | Amount/Batch |
|---|---|---|---|---|
| Item No. | Ingredient | % w/w | mg/g | (g) |
| 1 | Poloxamer F127 | 16.00 | 160.00 | 80.00 |
| | (Portion A) | | | |
| 2 | Poloxamer F127 | 0.50 | 5.00 | 2.50 |
| | (Portion B) | | | |
| 3 | Xanthan Gum, USP | 2.00 | 20.00 | 10.00 |
| 4 | Diclofenac Sodium | 2.00 | 20.00 | 10.00 |
| 5 | Citric Acid | 0.60 | 6.00 | 3.00 |
| | Monohydrate | | | |
| 6 | Sodium Citrate | 0.55 | 5.50 | 2.75 |
| | Dihydrate | | | |
| 7 | Benzyl Alcohol | 1.00 | 10.00 | 5.00 |
| 8 | Purified Water | 73.35 | 733.50 | 366.75 |
| 9 | Purified Water QS | 4.00 | 40.00 | 20.00 |
| 10 | Total | 100.00 | 1000.00 | 500.00 |

TABLE 15
Composition of 3% Diclofenac Sodium Gel Batch 178003-2203

| | | | | Amount/Batch |
|---|---|---|---|---|
| Item No. | Ingredient | % w/w | mg/g | (g) |
| 1 | Poloxamer F127 | 16.00 | 160.00 | 80.00 |
| | (Portion A) | | | |
| 2 | Poloxamer F127 | 0.5 | 5.00 | 2.50 |
| | (Portion B) | | | |
| 3 | Xanthan Gum, USP | 2.00 | 20.00 | 10.00 |
| 4 | Diclofenac Sodium | 3.00 | 30.00 | 15.00 |
| 5 | Citric Acid | 0.60 | 6.00 | 3.00 |
| | Monohydrate | | | |
| 6 | Sodium Citrate | 0.55 | 5.50 | 2.75 |
| | Dihydrate | | | |
| 7 | Benzyl Alcohol | 1.00 | 10.00 | 5.00 |
| 8 | Purified Water | 72.35 | 723.50 | 361.75 |
| 9 | Purified Water QS | 4.00 | 40.00 | 20.00 |
| 10 | Total | 100.00 | 1000.00 | 500.00 |

### Viscosity

The viscosity was evaluated using a calibrated parallel plate oscillating rheometer, model MCR 102 by Anton Paar, and a PP 25 measuring device. A single point viscosity was measured in duplicate at 0.22 l/s for 60 seconds at 25° C. and 37° C. Due to excessive air in the drug product a portion of each batch was centrifuged at 6,000 RPM for 10 minutes prior to measuring viscosity. The results outlined in 15 presents the average viscosity. Batches 178003-2201 and 178003-2202 exhibited a significant decrease in viscosity at 37° C. compared to the viscosity at 25° C. Batch 178003-2203 exhibited some thermo-reversible gel properties with a 17% increase in viscosity at 37° C.

The relationship (correlation) between the temperature and viscosity of the composition was important. Normally, viscosity of poloxamer gels increases with temperature. However, with the compositions described herein, the viscosity levels off or decreases. Without being bound by theory, it is believed this may be related to viscosity at room temperature (e.g., gel having higher viscosity at room temperature is more likely to have velocity decrease with increased temperature).

TABLE 16
Results for Viscosity at 0.22 1/s

| | | | Average | Average |
|---|---|---|---|---|
| | | | Viscosity at | Viscosity at |
| | Shear Rate | Duration | 25° C. | 37° C. |
| Batch No: | 1/s | Seconds | mPa*s | mPa*s |
| 178003-2201 | 0.22 | 60.0 | 1,652,400 | 996,155 |
| (1% diclofenac) | | | | |
| 178003-2202 | 0.22 | 60.0 | 895,255 | 827,575 |
| (2% diclofenac) | | | | |
| 178003-2203 | 0.22 | 60.0 | 953,035 | 1,135,450 |
| (3% diclofenac) | | | | |

pH

Due to the viscous nature of the drug product, pH was evaluated by diluting a portion of the drug product with an equal amount by weight of Total Organic Carbon (TOC) water and mixed until uniform. The pH results for each prototype are presented in Table 17, below. These results demonstrated a significant increase in pH with the increase in diclofenac sodium concentration. In some embodiments, this correlation may be due to the chemical nature of the ingredients.

Diclofenac solubility was shown to depend on pH, with higher solubility correlated with higher pH of the gel.

Due to the natural acidity of the vagina, vaginal compositions typically aim for a pH of about 4.5. Vaginal compositions described herein (e.g., using diclofenac) may have pH values up to about 5.5. In some embodiments, gels described herein containing diclofenac may require additional buffer (e.g., increased sodium citrate concentration) as compared to the gel containing other active agents.

TABLE 17
Results for pH

| Batch | pH |
|---|---|
| 178003-2201 | 5.04 |
| (1% diclofenac) | |
| 178003-2202 | 5.84 |
| (2% diclofenac) | |
| 178003-2203 | 6.54 |
| (3% diclofenac) | |

### Appearance

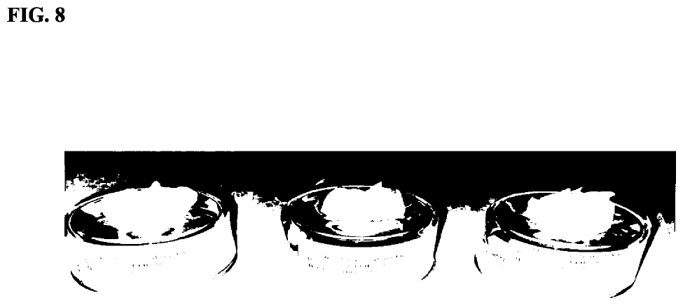

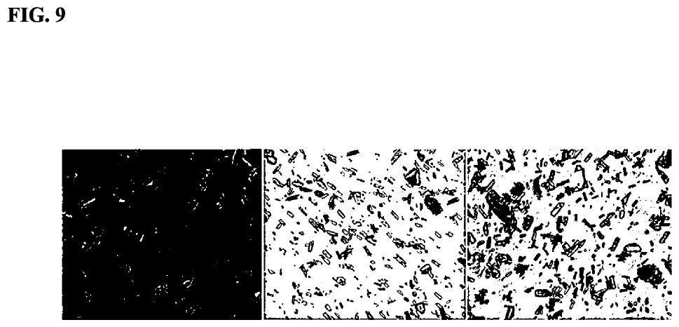

Appearance was evaluated visually based on color and general physical properties such as absence of agglomerates and absence of particulate matter. The results for appearance are presented in Table 18 and FIG. 8 and FIG. 9.

TABLE 18
Appearance results

| | Appearance | | | |
|---|---|---|---|---|
| | | | Absence of | |
| | | Absence of | Particulate | |
| Batch No. | Color | Agglomerates | Matter | Comments |
| 178003-2201 | Off White/ | Conforms | Conforms | Contains |
| (1% diclofenac) | Opaque | | | excessive |
| | | | | amounts of air |
| 178003-2202 | White/ | Conforms | Conforms | Contains |
| (2% diclofenac) | Slightly | | | excessive |
| | Opaque | | | amounts of air |
| 178003-2203 | White | Conforms | Conforms | Contains |
| (3% diclofenac) | | | | excessive |
| | | | | amounts of air |

The rheological comparison between the three prototypes indicated that batch 178003-2203 exhibited some thermos-reversible gel properties with a 17% increase in the average viscosity at 37° C. Variation in pH was reported between prototypes but was expected due to the chemical nature of the API. Appearance results indicated a slight difference in color between prototypes with batch 178003-2203 exhibiting a more pronounced white due to the higher concentrations of API.

Based on these data Diclofenac Sodium is compatible with the Gel Formulation 1 composition.

### Example 8: Uniformity and Viscosity

The results for assay of diclofenac sodium and benzyl alcohol were within the expected range. Uniformity for batches 2201 (1% diclofenac gel) and 2203 (3% diclofenac gel) was similar to the values obtained for assay and were within 2% RSD which is the verified precision of the method. Changes were observed in the rheology which correlated to the increased API (diclofenac) concentration. Particularly, these effects were observed in the shear stress of the flow point and in the thermal viscosity. These changes may be due to differences in the solids content and/or the water in the formulation. Surprisingly, a composition having diclofenac in suspension (not solution), was expected to take longer to release an equivalent amount of gel matrix. Also, a pH effect on release rate described herein. It is implicit in the data in Table 4 showing a non-linear variance in release rate as the API concentration goes up. When combined with the data in Table 17 that the pH goes up as the API concentration goes up, it supports the conclusion that higher pH supports faster release. Table 5 further supports this by showing that the 3% gel releases 50%, 80% and 100% faster than 1% or 2% diclofenac compositions.

### Uniformity

Uniformity was evaluated and the results are provided in Table 19 below

TABLE 19
uniformity results

| | Assay of | | Assay of | | |
|---|---|---|---|---|---|
| | Diclofenac | | Benzyl | % Related | |
| Batch Number | Sodium | Uniformity | Alcohol | Compounds | pH |
| 178003-2201 | 99.4% | 96.0% | 105.5% | Indv. Imp: ND | 5.0 |
| (1% diclofenac) | | 95.6% | | Total Imp: ND | |
| | | 95.6% | | | |
| | %RSD | 0.2% | | | |
| 178003-2202 | 97.0% | 90.4% | 102.6% | Indv. Imp: ND | 5.8 |
| (2% diclofenac) | | 99.0% | | Total Imp: ND | |
| | | 91.3% | | | |
| | %RSD | 5.1% | | | |
| 178003-2203 | 96.7% | 96.3% | 102.4% | Indv. Imp: ND | 6.5 |
| (3% diclofenac) | | 97.5% | | Total Imp: ND | |
| | | 95.3% | | | |
| | %RSD | 1.1% | | | |

### Rheology

Testing was performed at 25° C., 10 rad/s, from 0.1-100% amplitude (logarithmic ramp) and results are shown in Table 20, below.

TABLE 20
Rheology

| | LVER G' | LVER | Flow Point (Pa) | |
|---|---|---|---|---|
| Batch Number | (Pa) | G'/G” (Pa) | Tau (Pa) | G' (Pa) |
| 178003-2201 | 8790 | 9.1 | 307 | 2286 |
| (1% diclofenac) | | | | |
| 178003-2202 | 10100 | 9.1 | 276 | 2294 |
| (2% diclofenac) | | | | |
| 178003-2203 | 9930 | 11.0 | 215 | 2304 |
| (3% diclofenac) | | | | |

### Thermal Viscosity

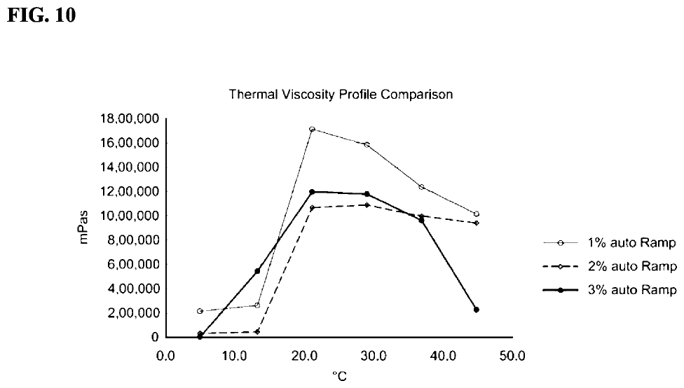

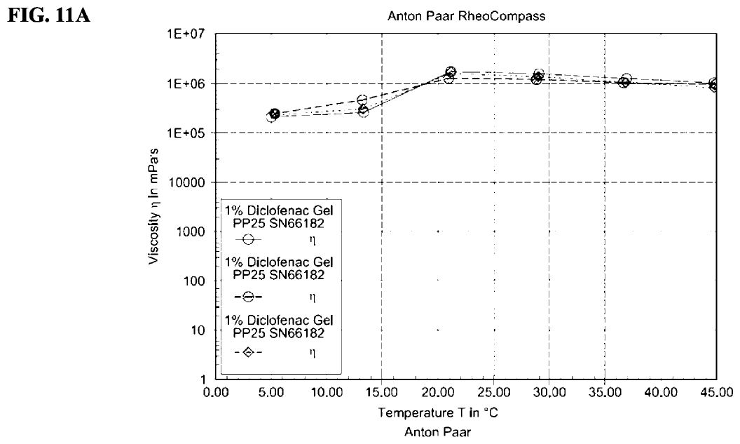

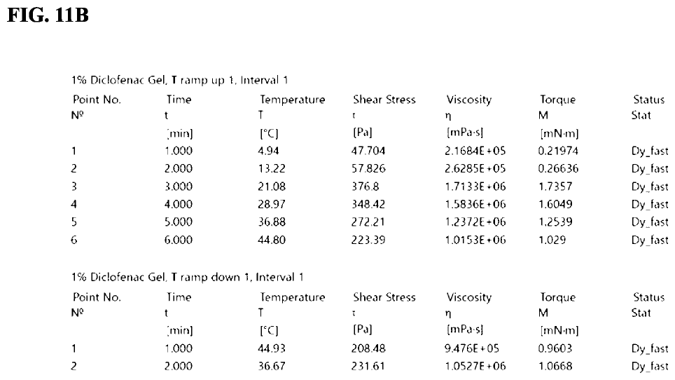

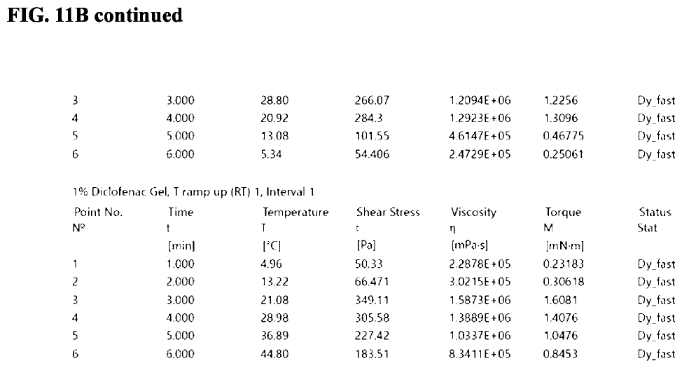

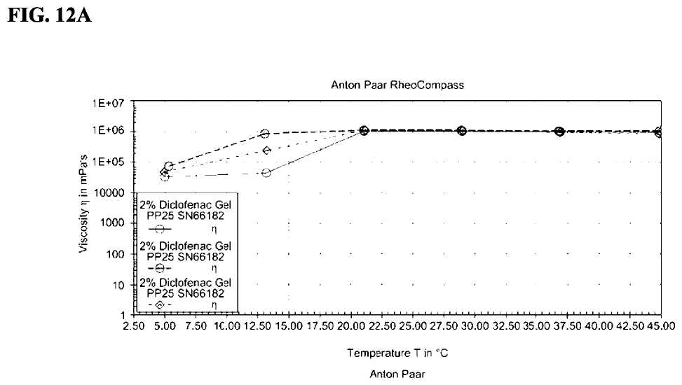

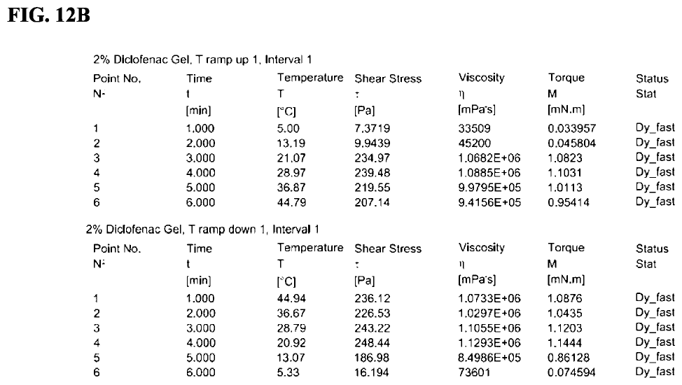

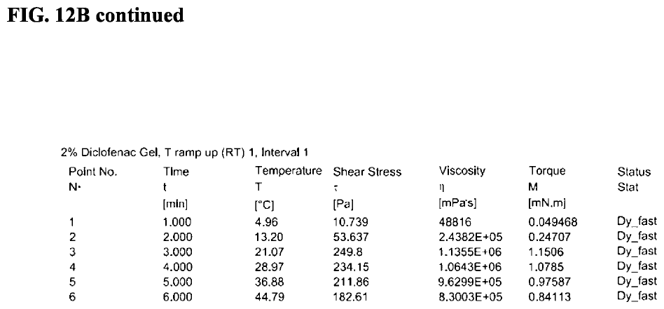

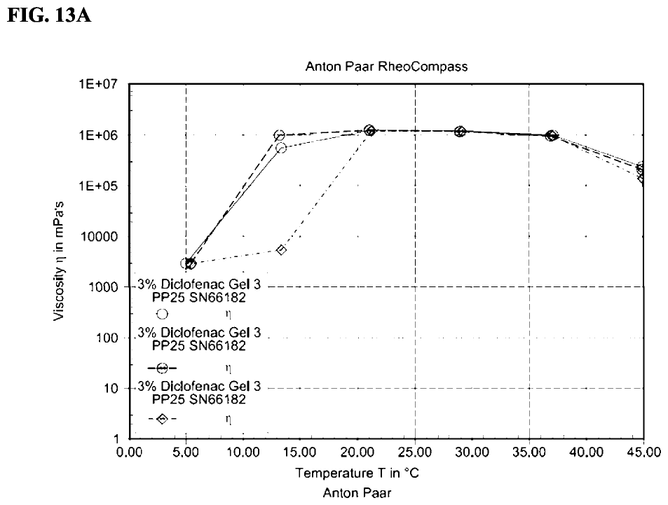

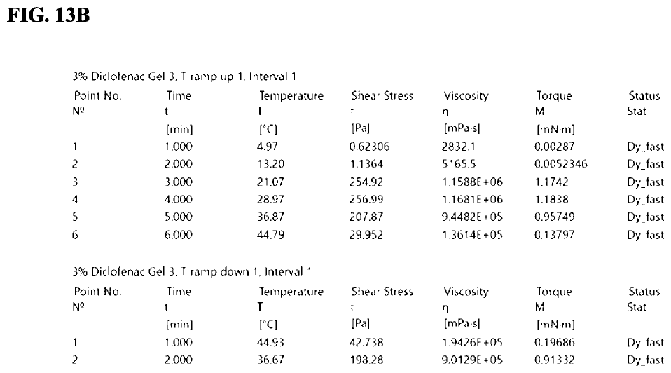

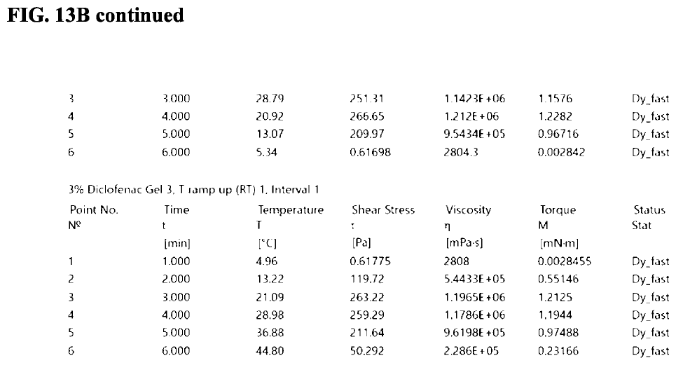

Thermal viscosity testing was performed at 0.22l/s, 5° C.-45° C., at 1-minute intervals, pre shear 3 l/s at 60 seconds and results are shown in FIG. 10 and Table 21 below. Thermal ramp up reports are shown in FIGS. 11A-11B, 12A-12B, and 13A-13B.

Surprisingly, the viscosity of the gel containing diclofenac was less viscous at room temperature than the gel alone or with other active ingredients. For example, the viscosity of the gel containing clindamycin at 25° C. is about 1,798,000 cP.

TABLE 21
Thermal viscosity

| | mPa*s | | |
|---|---|---|---|
| ° C. | 1% | 2% | 3% |
| 4.9 | 216,800 | 33,509 | 2,808 |
| 13.2 | 262,850 | 45,200 | 544,330 |
| 21.1 | 1,713,300 | 1,068,200 | 1,196,500 |
| 29.0 | 1,583,600 | 1,088,500 | 1,178,600 |
| 36.9 | 1,237,200 | 997,950 | 961,980 |
| 44.8 | 1,015,300 | 941,560 | 228,600 |

### Example 9: Air Removal

In this example, a manufacturing process is outlined describing air removal from the composition, as well as filing the composition into vaginal applicators is provided.

During mixing, 2000 g of benzyl alcohol and 33.0 kg of poloxamer 407 were added. Cooling and mixing continued until the solution was clear and the poloxamer was visually dissolved. The batch temperature reached approximately 11° C. during this step. This step created foam on the surface of the batch and the mixing speed was decreased to allow the foam to dissipate. Mixing speed was then increased, eventually reaching 1200 rpm for addition of the 4.00 kg of xanthan gum. After the addition of the xanthan gum, the product rapidly builds viscosity and the original mixing blade size proved inadequate. A switch was made to a 12-inch blade to better facilitate mixing at this stage of the process.

After this step in the process, the product generated a large amount of foaming. Initially, the product was transferred into a cooling, jacketed vacuum vessel and recirculated the product for several passes to help remove the air. A second approach used a Versator, which is a device especially designed to remove air from liquids and semi-solids. The product was passed through the Versator several times until all air was removed. Measurements of the viscosity after Versator treatment rose from 1.2 Cp (prior to use of the Versator) to over 3M Cp at room temperature.

Following this step, the product can be manually or filled using an automated filling machine. In some embodiments, the automated filling machine (e.g., a Capmatic automated filing machine) is especially designed to fill vaginal applicator-based products. As the viscosity of the product decreased at refrigerated temperatures, e.g., at temperatures of about 4° C. (e.g., a reduction in viscosity of 2-3×) provides a product suitable for automated filling into a vaginal applicator. In some embodiments, a cooled, jacketed vessel, feeding cooled product into the filler hopper to fill the applicators is used.

### Example 10: PH Effects on Release Rate

It was expected that the 2% diclofenac composition would release about 2× the amount of drug at a given time compared to the 1%, or conversely 3× that of the 3%. Surprisingly, however this was not observed. The higher concentration released much faster, and in fact the 2% diclofenac composition released all the active component faster than the 1% diclofenac composition released all of its active component.

The purpose of this study was to evaluate effect of the matrix pH on the release rate (diffusion) of diclofenac from the drug product.

### Objectives

Prepare 3× 100 g batches of Diclofenac 3% Gel at pH 3.5, 5.5, and 6.5
Evaluate the release rate of each pH adjusted batch by IVRT as per the test method RD-0240 over 24 hours.
Perform IVRT in simulated vaginal fluid for pH 3.5 and 5.5 over 24 hours.
Determine the effects of pH on the release rate of Diclofenac Vaginal Gel.

### Study Design

| Spl. ID# | Receptor Media | Drug Product pH |
|---|---|---|
| 2215-01 | 50 mM Phosphate Buffer pH 7.0 | 3.5 |
| 2215-02 | 50 mM Phosphate Buffer pH 7.0 | 5.5 |
| 2215-03 | 50 mM Phosphate Buffer pH 7.0 | 6.5 |
| 2215-04 | Simulated Vaginal Fluid pH 4.5 | 3.5 |
| 2215-05 | Simulated Vaginal Fluid pH 4.5 | 6.5 |

Testing

Assay of Diclofenac: 90.0-110.0% of LC
IVRT: Report Release Rate μg/hr/cm²
Rheology: Thermal Rheology, Amplitude Sweep, Flow Curve
pH: Report Results
Appearance: Report Results

The results for the assay of diclofenac and benzyl alcohol were within the range of 90.0-110.0%. Related compounds/impurities evidenced no trend in relation to pH. The results for amplitude sweep evidenced no statistically significant difference in the viscoelastic properties of the drug product over the evaluated pH range (pH 3.5-6.5). There was no statistically significant trend observed between thermal viscosity and the drug product's pH.

The solubility of the API (diclofenac) was assessed in pH buffered water in the same pH range as the test batches (pH 3.5 to 6.5) and a trend in relation to pH and API solubility was observed (Table 22A), demonstrating an exponential increase over the evaluated pH range.

TABLE 22A
Assay Results for pH Adjusted Diclofenac Vaginal Gel 3%

| | | % | | | | |
|---|---|---|---|---|---|---|
| | | Assay | % Related Compounds | | | |
| | | of | Unk | Total | Diclofenac | |
| | % Assay of | Benzyl | RRT | % | Solubility | Measured |
| Batch No. | Diclofenac | Alcohol | 0.74 | Imp | (μg/mL) | pH |
| 2218-01 (pH 3.5) | 98.0 | 104.8 | ND | ND | 1.0 | 3.6 |
| 178004-2209 (pH 4.5) | 98.3 | 100.2 | 0.11 | 0.11 | 5.0 | 4.6 |
| 2218-02 (pH 5.5) | 105.1 | 101.7 | <LOQ | <LOQ | 61.0 | 5.7 |
| 2218-03 (pH 6.5) | 104.6 | 104.4 | <LOQ | <LOQ | 547.0 | 6.5 |

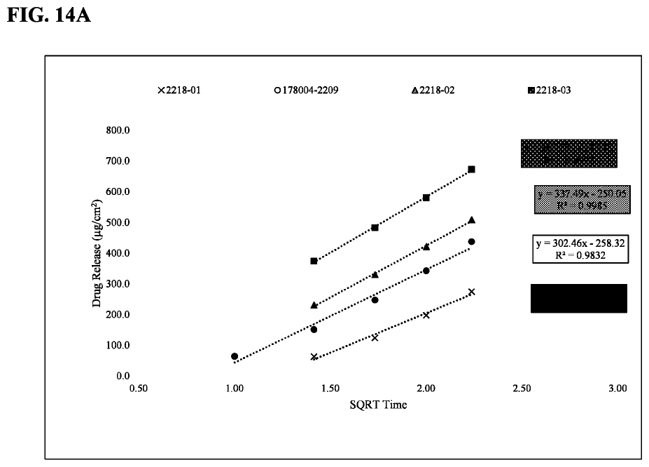

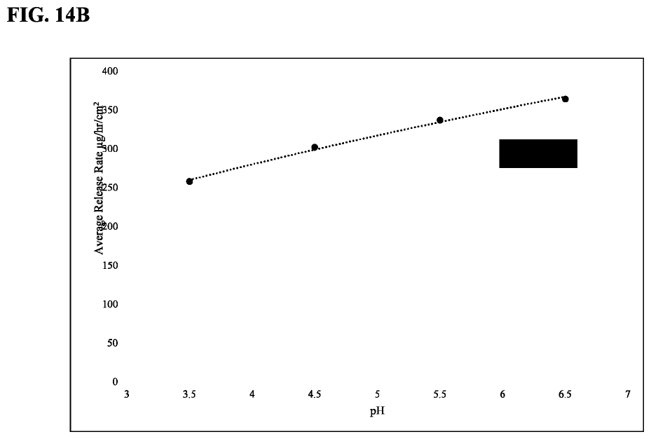

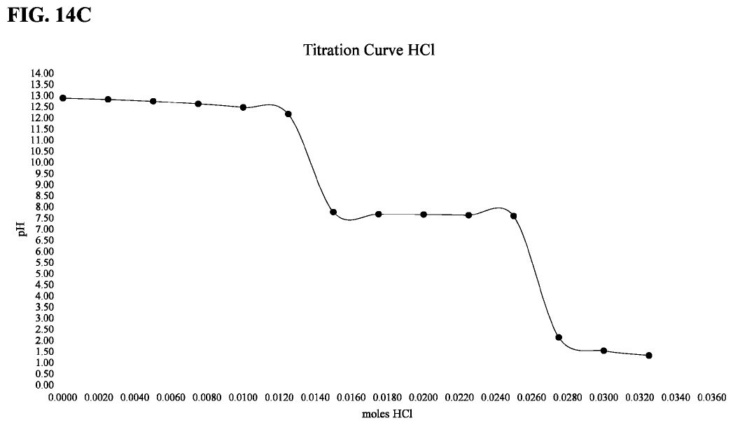

A trend between the diclofenac vaginal gel pH and drug release was identified. As the pH of the drug product increased the average drug release rate proportionally increased (Table 22B, FIGS. 14A and 14B). Statistical analysis was performed on the drug release of diclofenac vaginal gel at pH 3.5, 4.5, 5.5, and 6.5 using Minitab™ statistical software. The probability plot indicated a normal distribution of these data with a p-value of 0.081 and an Anderson Darling statistic of 0.646. Regression analysis for the relationship between drug release and pH identified a positive correlation between the increasing pH of diclofenac vaginal gel and the increasing average drug release rate (r=0.99).

TABLE 22B
Assay Results for pH Adjusted Diclofenac Vaginal Gel 3%

| | | Average Drug Release (μg/cm²) | | | |
|---|---|---|---|---|---|
| Time | SQRT | 2218-01 | 178004-2209 | 2218-02 | 2218-03 |
| (hours) | Time | (pH 3.5) | (pH 4.5) | (pH 5.5) | (pH 6.5) |
| 1.0 | 1.00 | 28.7 | 64.4 | 119.4 | 231.2 |
| 2.0 | 1.41 | 63.3 | 151.3 | 231.1 | 373.9 |
| 3.0 | 1.73 | 124.5 | 247.2 | 329.8 | 483.5 |
| 4.0 | 2.00 | 198.5 | 343.0 | 421.6 | 580.9 |
| 5.0 | 2.24 | 275.3 | 437.8 | 508.8 | 673.3 |

| | | | | |
|---|---|---|---|---|
| Slope (Rate) | 257.7 | 302.5 | 337.5 | 363.6 |
| R² | 0.986 | 0.983 | 0.998 | 0.999 |
| %RSD | 9.3 | 5.7 | 8.9 | 1.9 |

### Example 11: pH Adjustment Evaluation

The purpose of this study is to evaluate of hydrochloric acid needed to neutralize the basic properties of the sodium salt for the diclofenac; and achieve a pH of 4.5 for the final drug product. 5N Hydrochloric Acid (HCl) and 5N Sodium Hydroxide (NaOH) were prepared and a pH curve of 4% diclofenac sodium in water was observed in Table 22C.

TABLE 22C
Results for pH Curve of Diclofenac Sodium

| Step | 5N NaOH | 5N HCl | Total | | |
|---|---|---|---|---|---|
| No. | added (mL) | added (mL) | (mL) | moles | pH |
| 1 | 0.0 | 0.0 | 0.0 | 0.0000 | 7.85 |
| 2 | 1.0 | 0.0 | 1.0 | 0.0050 | 12.50 |
| 3 | 0.5 | 0.0 | 1.5 | 0.0075 | 12.65 |
| 4 | 0.5 | 0.0 | 2.0 | 0.0100 | 12.75 |
| 5 | 0.5 | 0.0 | 2.5 | 0.0125 | 12.83 |
| 6 | 0.5 | 0.0 | 3.0 | 0.0150 | 12.89 |
| 7 | 0.0 | 0.0 | 0.0 | 0.0000 | 12.89 |
| 8 | 0.0 | 0.5 | 0.5 | 0.0025 | 12.83 |
| 9 | 0.0 | 0.5 | 1.0 | 0.0050 | 12.75 |
| 10 | 0.0 | 0.5 | 1.5 | 0.0075 | 12.63 |
| 11 | 0.0 | 0.5 | 2.0 | 0.0100 | 12.47 |
| 12 | 0.0 | 0.5 | 2.5 | 0.0125 | 12.17 |
| 13 | 0.0 | 0.5 | 3.0 | 0.0150 | 7.77 |
| 14 | 0.0 | 0.5 | 3.5 | 0.0175 | 7.68 |
| 15 | 0.0 | 0.5 | 4.0 | 0.0200 | 7.66 |
| 16 | 0.0 | 0.5 | 4.5 | 0.0225 | 7.63 |
| 17 | 0.0 | 0.5 | 5.0 | 0.0250 | 7.59 |
| 18 | 0.0 | 0.5 | 5.5 | 0.0275 | 2.14 |
| 19 | 0.0 | 0.5 | 6.0 | 0.0300 | 1.54 |
| 20 | 0.0 | 0.5 | 6.5 | 0.0325 | 1.33 |

The titration curve (FIG. 14C) was used to determine the buffering capacity of the diclofenac and the amount of 5N HCl to neutralize the diclofenac. Based on these data the diclofenac showed 2 equivalence points with a pKa of ~7.7. The buffering capacity (moles/mole) to adjust the pH by 1 unit was determined to be 0.625 mMoles of strong acid or base.

The pH of diclofenac in water is around 7.8; from this the titration curve was used to determine the moles of HCl to neutralize diclofenac and obtain a pH of 3.8. The molar ratio (diclofenac: HCl) is (1.023) and the amount of acid to neutralize the diclofenac can be calculated as: Grams diclofenac/(318.13 g/mole*1.023)/5*1000=mL of 5N HCl

For diclofenac formulations, 8.50e10-5 moles (~0.62 mL) of 5N HCl is added per gram of diclofenac sodium to achieve a pH of 3.8.

### Example 12: R&D Stability

Two formulation of diclofenac vaginal gel (1% and 5%) and one placebo were prepared and evaluated over 3 months at 25° C./60% relative humidity (RH) and 40° C./75% RH stored upright. All formulations were tested at time zero, 1 month, and 3 months.

TABLE 23
Batch 178004-2212 Diclofenac Vaginal Gel 1% Formulation Composition

| Item No. | Ingredient | % w/w | mg/g |
|---|---|---|---|
| 1 | Poloxamer 407, USP (Portion A) | 16.00 | 160.00 |
| 2 | Poloxamer 407, USP (Portion A) | 0.50 | 5.00 |
| 3 | Xanthan gum, USP | 2.00 | 20.00 |
| 4 | Diclofenac Sodium* | 1.07 | 10.72 |
| 5 | Citric acid monohydrate, USP | 0.90 | 9.00 |
| 6 | Sodium citrate dihydrate, USP | 0.83 | 8.25 |
| 7 | Benzyl Alcohol, USP | 1.00 | 10.00 |
| 8 | 5N HCl | 0.11 | 1.12 |
| 9 | Purified Water, USP | 70.09 | 700.91 |
| 10 | Purified Water QS | 7.50 | 75.00 |
| | Total | 100.00 | 1000.00 |

*Amount adjusted for Diclofenac free base and purity.

TABLE 24
Batch 178004-2213 Diclofenac Vaginal Gel 5% Formulation Composition

| Item No. | Ingredient | % w/w | mg/g |
|---|---|---|---|
| 1 | Poloxamer 407, USP (Portion A) | 16.00 | 160.00 |
| 2 | Poloxamer 407, USP (Portion A) | 0.50 | 5.00 |
| 3 | Xanthan gum, USP | 2.00 | 20.00 |
| 4 | Diclofenac Sodium* | 5.36 | 53.60 |
| 5 | Citric acid monohydrate, USP | 0.90 | 9.00 |
| 6 | Sodium citrate dihydrate, USP | 0.83 | 8.25 |
| 7 | Benzyl Alcohol, USP | 1.00 | 10.00 |
| 8 | 5N HCl | 0.56 | 5.62 |
| 9 | Purified Water, USP | 65.35 | 653.53 |
| 10 | Purified Water QS | 7.50 | 75.00 |
| | Total | 100.00 | 1000.00 |

*Amount adjusted for Diclofenac free base and purity.

TABLE 25
Batch 178004-2214P Vaginal Gel Placebo Formulation Composition

| Item No. | Ingredient | % w/w | mg/g |
|---|---|---|---|
| 1 | Poloxamer 407, | 16.50 | 165.00 |
| | USP (Portion A) | | |
| 2 | Xanthan gum, USP | 2.00 | 20.00 |
| 3 | Citric acid monohydrate, | 0.90 | 9.00 |
| | USP | | |
| 4 | Sodium citrate dihydrate, | 0.83 | 8.25 |
| | USP | | |
| 5 | Benzyl Alcohol, USP | 1.00 | 10.00 |
| 6 | 5M HCl | QS to pH 4.5 | QS to pH 4.5 |
| 7 | Purified Water, USP | 71.28 | 712.75 |
| 8 | Purified Water QS | 7.50 | 75.00 |
| | Total | 100.00 | 1000.00 |

The samples will be stored at the upright under the following conditions for 3 months.

25° C./60% RH: 25 applicators total (for each active formulation)
7 applicators at T=0 and 3 months, 5 applicators at 1-month analytical testing
6 contingency applicators
Placebo: 3 applicators per time point for analytical testing and 1 contingency applicator

40° C./75% RH: 25 applicators total (for each active formulation)
7 applicators at T=0 and 3 months, 5 applicators at 1-month analytical testing
6 contingency applicators
Placebo: 3 applicators per time point for analytical testing and 1 contingency applicator

TABLE 26
Storage Stability Schedule and Testing Schedule

| | | Time Point | | | |
|---|---|---|---|---|---|
| Test | Test Method | T-0 | 1M | 3M | Target Criteria |
| Assay of Diclofenac | RD-0237 V1 | X | A, B | A, B | 90.0-110.0% of LC |
| Absence of Active | RD-0237 V1 | X | C, D | C, D | Absent for Diclofenac |
| USP Content Uniformity | RD-0240 V1 | X | — | — | Report average, % RSD |
| | | | | | and AV value at n = 10 |
| Related Compounds | RD-0237 V1 | X | A, B, C, D | A, B, C, D | Report Results >0.05%, NMT |
| | | | | | 0.5% RCA, NMT 0.2% for any |
| | | | | | unspecified imp, NMT 1.0% |
| | | | | | total impurities |
| Assay of Benzyl Alcohol | RD-0237 V1 | X | A, B C, D | A, B, C, D | 90.0-110.0% |
| Benzaldehyde | RD-0237 V1 | X | — | A,B, C, D | Report Results (for information only) |
| IVRT | RD-0240 V1 | X | — | A, B | Report Results, NMT = 15% from T = 0 |
| Viscosity | Rotational Rheometery | X | A ,B, C, D | A, B, C, D | Report Results |
| Thermal Viscosity | Rotational Rheometery | X | — | A, B, C, D | Report Results |
| Flow Point | Oscillating Rheometery | X | — | A, B, C, D | Report Tau and Viscosity at the |
| | | | | | flow point |
| L VER | Oscillating Rheometery | X | — | A, B, C, D | Report Tau and Viscosity |
| pH | USP<791> | X | A, B, C, D | A, B, C, D | Report Results |
| Appearance | Visual | X | A, B, C, D | A, B, C, D | White off white gel with no |
| | | | | | indication of phase separation, |
| | | | | | agglomerates, or particulate matter |
| PSD | RD-0254 V1 | X | A, B | A, B | Report d10, d50, and d90 |
| Density | Pycnometery | X¹ | — | — | Report Results |

1: perform on the bulk drug product
X: Initial
A: 25° C./60% RH (Active)
B: 40° C./75% RH (Active)
C: 25° C./60% RH (Placebo)
D: 40° C./75% RH (Placebo)

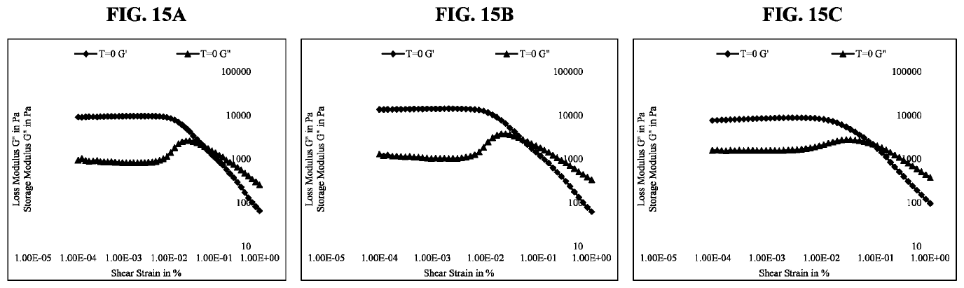

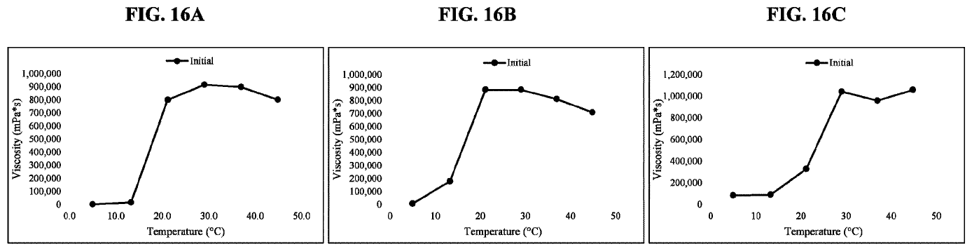

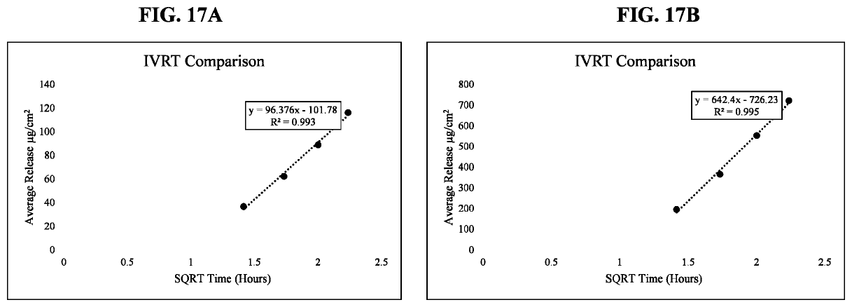

R&D stability batches were evaluated for assay of diclofenac (Table 27), uniformity (Tables 28 and 29), benzyl alcohol (Table 30), benzaldehyde (Table 31), amplitude sweep (Table 32 and FIG. 15A-15C), thermal viscosity (Tables 33-35 and FIG. 16A-16C), single point viscosity (Table 36), IVRT (Tables 37-39 and FIG. 17A-17B), pH and appearance (Table 40), and density (Table 41) for batch 178004-2212 (1% diclofenac gel composition), batch 178004-2213 (5% diclofenac gel composition), batch 178004-2214P (placebo gel composition).

The rheological method for diclofenac vaginal gel was evaluated using a calibrated parallel plate oscillating rheometer, model MCR 102 by Anton Paar, and a PP25 measuring device at 25° C. The specific tests used to evaluate the rheological properties of the drug product were amplitude sweep, single point viscosity, and thermal rheology.

Amplitude sweep uses controlled strain with increasing amplitude to determine the product's linear viscoelastic region (LVER). The LVER indicates the range in which the test can be carried. The viscoelastic behavior of a sample can be characterized by the storage modulus G′ and the loss modulus G″ in the LVER region. The storage modulus G′ relates to the materials ability to store energy elastically. The loss modulus G″ of a material is the ratio of the viscous component to the stress and is related to the material's ability to dissipate stress through heat.

Amplitude sweep test parameters:
Temperature: 25° C.; Plate: PP25; Data Points: 50; Wait/recovery time: 60 seconds
Amplitude—Variable: Shear Strain (Oscillating); Ramp Logarithmic Profile; Initial 1.00% and Final 100.00%
Frequency—Variable: Angular Frequency; Ramp Logarithmic Profile; Initial 1.0 (l/s) and Final 10.0 (l/s)

The single point viscosity of the drug product was performed at 10 l/s for 20 seconds. Thermal viscosity of the drug product was performed at 0.22 l/s from 5° C.-45° C., at 1-minute intervals, and a pre shear condition of 3 l/s for 60 seconds.

TABLE 27
Assay of Diclofenac Results for R&D Stability

| | | % Assay | % Assay Weight |
|---|---|---|---|
| Batch | Time Point | of LC | Adjusted |
| 178004-2212 | T = 0 | 101.3 | |
| | 1M 40° C./75% RH | 90.3 | 99.7 |
| | 3M 40° C./75% RH | | |
| | 1M 25° C./60% RH | 89.4 | 99.0 |
| | 3M 25° C./60% RH | | |
| 178004-2213 | T = 0 | 100.1 | |
| | 1M 40° C./75% RH | 96.1 | 99.9 |
| | 3M 40° C./75% RH | | |
| | 1M 25° C./60% RH | 100.1 | 100.2 |
| | 3M 25° C./60% RH | | |

Content uniformity was evaluated for batches 2212 and 2213 on 10 applicators (3 applicators from the beginning, 4 from the middle, and 3 from the end of fill), results in Table 28 and Table 29.

TABLE 28
178004-2212 Content Uniformity Results

| | Sample | % | Spl. | Weight |
|---|---|---|---|---|
| No. | Location | LC | Wght. (g) | Assay |
| 1 | Beginning | 88.9 | 2.275 | 97.7 |
| 2 | Beginning | 87.4 | 2.082 | 105.0 |
| 3 | Beginning | 88.3 | 2.275 | 97.0 |
| 4 | Middle | 92.2 | 2.332 | 98.9 |
| 5 | Middle | 90.6 | 2.306 | 98.2 |
| 6 | Middle | 92.3 | 2.236 | 103.2 |
| 7 | Middle | 91.7 | 2.301 | 99.6 |
| 8 | End | 91.9 | 2.301 | 99.8 |
| 9 | End | 92.0 | 2.279 | 100.9 |
| 10 | End | 91.6 | 2.303 | 99.4 |

| | | | |
|---|---|---|---|
| Average | 90.7 | 2.3 | 100.0 |
| StDev | 1.8 | 0.1 | 2.5 |
| % RSD | 2.0 | 3.1 | 2.5 |

TABLE 29
178004-2213 Content Uniformity Results

| No. | Sample Location | % LC | Sample Weight (g) |
|---|---|---|---|
| 1 | Beginning | 101.0 | 2.520 |
| 2 | Beginning | 101.3 | 2.524 |
| 3 | Beginning | 100.6 | 2.508 10 |
| 4 | Middle | 101.9 | 2.548 |
| 5 | Middle | 101.2 | 2.514 |
| 6 | Middle | 101.0 | 2.516 |
| 7 | Middle | 101.8 | 2.530 |
| 8 | End | 100.6 | 2.499 |
| 9 | End | 98.6 | 2.458 |
| 10 | End | 98.6 | 2.459 |

| | | |
|---|---|---|
| Average | 100.7 | 2.508 15 |
| StDev | 1.16 | 0.03 |
| % RSD | 1.15 | 1.16 |

TABLE 30
Benzyl Alcohol Results for R&D Stability

| Batch | Time Point | % Assay |
|---|---|---|
| 178004-2212 | T = 0 | 100.3 |
| | 1M 40° C./75% RH | 91.2 |
| | 3M 40° C./75% RH | |
| | 1M 25° C./60% RH | 96.2 |
| | 3M 25° C./60% RH | |
| 178004-2213 | T = 0 | 100.3 |
| | 1M 40° C./75% RH | 91.4 |
| | 3M 40° C./75% RH | |
| | 1M 25° C./60% RH | 96.3 |
| | 3M 25° C./60% RH | |
| 178004-2214P | T = 0 | 98.3 |
| | 1M 40° C./75% RH | 91.5 |
| | 3M 40° C./75% RH | |
| | 1M 25° C./60% RH | 96.7 |
| | 3M 25° C./60% RH | |

TABLE 31
Results for Benzaldehyde R&D Stability

| Batch | Time Point | Benzaldehyde (mg/g) |
|---|---|---|
| 178004-2212 | T = 0 | 0.019 |
| | 1M 40° C./75% RH | 0.019 |
| | 3M 40° C./75% RH | |
| | 1M 25° C./60% RH | 0.020 |
| | 3M 25° C./60% RH | |
| 178004-2213 | T = 0 | 0.027 |
| | 1M 40° C./75% RH | 0.025 |
| | 3M 40° C./75% RH | |
| | 1M 25° C./60% RH | 0.027 |
| | 3M 25° C./60% RH | |
| 178004-2214P | T = 0 | 0.019 |
| | 1M 40° C./75% RH | 0.018 |
| | 3M 40° C./75%RII | |
| | 1M 25° C./60% RH | 0.017 |
| | 3M 25° C./60% RH | |

TABLE 32
Amplitude Sweep Results R&D Stability

| | | LVER Limit | | Flow Point τ y | |
|---|---|---|---|---|---|
| | | Shear | Storage | Shear | Storage |
| | | Stress | Modulus | Stress | Modulus |
| Batch | | τ | G′ | τ | G′ |
| No. | Storage Condition | (Pa) | (Pa) | (Pa) | (Pa) |
| 178004- | T = 0 | 9.7 | 9,520 | 163.6 | 2,018 |
| 2212 | 1M 40° C./75% RH | NT | | | |
| | 1M 25° C./60% RH | NT | | | |
| | 3M 40° C./75% RH | | | | |
| | 3M 25° C./60% RH | | | | |
| 178004- | T = 0 | 14.4 | 14,100 | 194.7 | 2,981 |
| 2213 | 1M 40° C./75% RH | NT | | | |
| | 1M 25° C./60% RH | NT | | | |
| | 3M 40° C./75% RH | | | | |
| | 3M 25° C./60% RH | | | | |
| 178004- | T = 0 | 0.793 | 8,200 | 270.8 | 2,233 |
| 2214P | 1M 40° C./75% RH | NT | | | |
| | 1M 25° C./60% RH | NT | | | |
| | 3M 40° C./75% RH | | | | |
| | 3M 25° C./60% RH | | | | |

TABLE 33
178004-2212 Thermal Viscosity Results

| | mPa*s at 0.22 1/s | | |
|---|---|---|---|
| | | 3 Month | 3 Month |
| 0° C. | Initial | 40° C./75% RH | 25° C./60% RH |
| 4.9 | 1,242 | | |
| 13.2 | 15,256 | | |
| 21.1 | 800,280 | | |
| 29.0 | 915,110 | | |
| 36.9 | 898,405 | | |
| 44.8 | 800,930 | | |

TABLE 34
178004-2213 Thermal Viscosity Results

| | mPa*s at 0.22 1/s | | |
|---|---|---|---|
| | | 3 Month | 3 Month |
| ° C. | Initial | 40° C./75% RH | 25° C./60% RH |
| 4.9 | 5.947 | | |
| 13.2 | 178,984 | | |
| 21.1 | 883,350 | | |
| 29.0 | 883,560 | | |
| 36.9 | 811,865 | | |
| 44.8 | 709,250 | | |

TABLE 35
178004-2214P Thermal Viscosity Results

| | mPa*s at 0.22 1/s | | |
|---|---|---|---|
| | | 3 Month | 3 Month |
| ° C. | Initial | 40° C./75% RH | 25° C./60% RH |
| 4.9 | 84,520 | | |
| 13.2 | 90,733 | | |
| 21.1 | 328,280 | | |
| 29.0 | 1,042,950 | | |
| 36.9 | 959,495 | | |
| 44.8 | 1,058,900 | | |

TABLE 36
Results for Single Point Viscosity R&D Stability

| Batch | Time Point | Viscosity at 10 1/s (mPa*s) |
|---|---|---|
| 178004-2212 | T = 0 | 30,180 |
| | 1M 40° C./75% RH | |
| | 3M 40° C./75% RH | |
| | 1M 25° C./60% RH | |
| | 3M 25° C./60% RH | |
| 178004-2213 | T-0 | 32,850 |
| | 1M 40° C./75% RH | |
| | 3M 40° C./75% RH | |
| | 1M 25° C./60% RH | |
| | 3M 25° C./60% RH | |
| 178004-2214P | T-0 | 37,720 |
| | 1M 40° C./75% RH | |
| | 3M 40° C./75% RH | |
| | 1M 25° C./60% RH | |
| | 3M 25° C./60% RH | |

TABLE 37
178004-2212 Initial in vitro Release Testing (IVRT) Results R&D Stability

| | sqrt | μg/cm² | | | | | |
|---|---|---|---|---|---|---|---|
| Hours | (hrs) | 1 | 2 | 3 | 4 | 5 | 6 |
| 1.0 | 1.00 | — | 22.49 | 21.68 | 22.54 | 18.97 | 19.09 |
| 2.0 | 1.41 | — | 50.14 | 46.33 | 42.37 | 39.44 | 42.90 |
| 3.0 | 1.73 | — | 83.97 | 78.04 | 67.42 | 68.87 | 75.36 |
| 4.0 | 2.00 | — | 118.85 | 111.88 | 95.25 | 99.25 | 108.30 |
| 5.0 | 2.24 | — | 153.41 | 145.77 | 123.66 | 131.79 | 143.12 |

| | | | | | | |
|---|---|---|---|---|---|---|
| Slope | — | 106.2 | 100.6 | 81.7 | 91.3 | 100.4 |
| Rsq | — | 0.976 | 0.970 | 0.967 | 0.964 | 0.969 |

TABLE 38
178004-2213 Initial IVRT Results R&D Stability

| | sqrt | μg/cm² | | | | | |
|---|---|---|---|---|---|---|---|
| Hours | (hrs) | 1 | 2 | 3 | 4 | 5 | 6 |
| 1.0 | 1.00 | 95.94 | 87.18 | 87.73 | 93.17 | 77.33 | 81.52 |
| 2.0 | 1.41 | 211.65 | 198.93 | 186.78 | 210.21 | 188.21 | 182.18 |
| 3.0 | 1.73 | 395.93 | 372.29 | 338.45 | 383.06 | 370.05 | 336.25 |
| 4.0 | 2.00 | 606.27 | 561.10 | 512.14 | 574.28 | 558.26 | 507.06 |
| 5.0 | 2.24 | 798.84 | 724.86 | 681.38 | 747.15 | 715.68 | 663.76 |

| | | | | | | |
|---|---|---|---|---|---|---|
| Slope | 575.1 | 524.3 | 482.9 | 535.2 | 527.8 | 476.4 |
| Rsq | 0.993 | 0.965 | 0.956 | 0.965 | 0.967 | 0.962 |

TABLE 39
Particle Size Distribution (PSD)

| Batch | Time Point | d10 (μm) | d50 (μm) | d90 (μm) |
|---|---|---|---|---|
| 178004-2212 | T = 0 | 1.7 | 15.4 | 47.0 |
| | 1M 40° C./75% RH | 1.3 | 8.6 | 47.7 |
| | 3M 40° C./75% RH | | | |
| | 1M 25° C./60% RH | 1.7 | 12.7 | 42.1 |
| | 3M 25° C./60% RH | | | |
| 178004-2213 | T = 0 | 1.8 | 9.4 | 26.1 |
| | 1M 40° C./75% RH | 2.9 | 11.6 | 36.3 |
| | 3M 40° C./75% RH | | | |
| | 1M 25° C./60% RH | 2.4 | 10.9 | 33.4 |
| | 3M 25° C./60% RH | | | |

TABLE 40
pH and Appearance Results for R&D Stability

| | | | Appearance | | |
|---|---|---|---|---|---|
| | | | | Absence | Absence of |
| | | | | of | Agglomerates/ |
| | | | | Phase | Particulate |
| Batch | Time Point | pH | Color | Separation | Matter |
| 178004- | T = 0 | 4.3 | White | Conforms | Conforms |
| 2212 | 1M 40° C./75% RH | 4.3 | White | Conforms | Conforms |
| | 3M 40° C./75% RH | | | | |
| | 1M 25° C./60% RH | 4.3 | White | Conforms | Conforms |
| | 3M 25° C./60% RH | | | | |
| 178004- | T = 0 | 4.3 | White | Conforms | Conforms |
| 2213 | 1M 40° C./75% RH | 4.4 | White | Conforms | Conforms |
| | 3M 40° C./75% RH | | | | |
| | 1M 25° C./60% RH | 4.3 | White | Conforms | Conforms |
| | 3M 25° C./60% RH | | | | |
| 178004- | T = 0 | 4.5 | Clear | Conforms | Conforms |
| 2214P | 1M 40° C./75% RH | 4.6 | Clear | Conforms | Conforms |
| | 3M 40° C./75% RH | | | | |
| | 1M 25° C./60% RH | 4.6 | Clear | Conforms | Conforms |
| | 3M 25° C./60% RH | | | | |

TABLE 41
Density Results for R&D Stability

| Batch | Time Point | Density (g/mL) |
|---|---|---|
| 178004-2212 | Initial | 1.01 |
| 178004-2213 | Initial | 1.02 |
| 178004-2214P | Initial | 1.00 |

### Example 13: Formulations for Animal Toxicology Studies

Two formulations of diclofenac vaginal gel and one placebo were prepared in support of animal toxicology studies. Each batch was evaluated for assay, related compounds, preservatives, viscosity, pH, and appearance. Batch preparation was performed according to the methods described above.

TABLE 42
Formulation Composition of Diclofenac Vaginal Gel
10% Batch 178004-2206

| | | | | Amount/ |
|---|---|---|---|---|
| Item | | | | Batch |
| No. | Ingredient | % w/w | mg/g | (g) |
| 1 | Poloxamer 407, USP (Portion A) | 16.00 | 160.00 | 40.00 |
| 2 | Poloxamer 407, USP (Portion A) | 0.50 | 5.00 | 1.25 |
| 3 | Xanthan gum, USP | 2.00 | 20.00 | 5.00 |
| 4 | Diclofenac Sodium* | 10.77 | 107.75 | 26.94 |
| 5 | Citric acid monohydrate, USP | 0.90 | 9.00 | 2.25 |
| 6 | Sodium citrate dihydrate, USP | 0.83 | 8.25 | 2.06 |
| 7 | Benzyl Alcohol, USP | 1.00 | 10.00 | 2.50 |
| 8 | Purified Water, USP | 64.00 | 640.00 | 160.00 |
| 9 | Purified Water QS | 4.00 | 40.00 | 10.00 |
| | Total | 100.00 | 1000.00 | 250.00 |

*Amount adjusted for Diclofenac free base and purity.

TABLE 43
Formulation Composition of Diclofenac Vaginal Gel 3% Batch 178004-2209

| | | | | Amount/ |
|---|---|---|---|---|
| Item | | | | Batch |
| No. | Ingredient | % w/w | mg/g | (g) |
| 1 | Poloxamer 407, USP | 16.00 | 160.00 | 40.00 |
| | (Portion A) | | | |
| 2 | Poloxamer 407, USP | 0.50 | 5.00 | 1.25 |
| | (Portion B) | | | |
| 3 | Xanthan gum, USP | 2.00 | 20.00 | 5.00 |
| 4 | Diclofenac Sodium* | 3.23 | 32.32 | 8.08 |
| 5 | Citric acid monohydrate, | 0.90 | 9.00 | 2.25 |
| | USP | | | |
| 6 | Sodium citrate dihydrate, | 0.83 | 8.25 | 2.06 |
| | USP | | | |
| 7 | Benzyl Alcohol, USP | 1.00 | 10.00 | 2.50 |
| 8 | 5N HCl | QS to | QS to | QS to |
| | | pH 4.2 | pH 4.2 | pH 4.2 |
| 9 | Purified Water, USP | 70.54 | 705.43 | 176.36 |
| 10 | Purified Water QS | 5.00 | 50.00 | 12.50 |
| | Total | 100.00 | 1000.00 | 250.00 |

*Amount adjusted for Diclofenac free base and purity.

TABLE 44
Formulation Composition of Vaginal Gel Placebo Batch 178004-2210P

| | | | | Amount/ |
|---|---|---|---|---|
| Item | | | | Batch |
| No. | Ingredient | % w/w | mg/g | (g) |
| 1 | Poloxamer 407, USP | 16.50 | 165.00 | 41.25 |
| | (Portion A) | | | |
| 2 | Xanthan gum, USP | 2.00 | 20.00 | 5.00 |
| 3 | Citric acid monohydrate, | 0.90 | 9.00 | 2.25 |
| | USP | | | |
| 4 | Sodium citrate dihydrate, | 0.83 | 8.25 | 2.06 |
| | USP | | | |
| 5 | Benzyl Alcohol, USP | 1.00 | 10.00 | 2.50 |
| 6 | 5N HCl | QS to | QS to | QS to |
| | | pH 4.2 | pH 4.2 | pH 4.2 |
| 7 | Purified Water, USP | 73.78 | 737.75 | 184.44 |
| 8 | Purified Water QS | 5.00 | 50.00 | 12.50 |
| | Total | 100.00 | 1000.00 | 250.00 |

Toxicology batches were evaluated for assay of diclofenac as described above. Placebo batch 2210P was evaluated for absence of diclofenac. These data (Table 45) were within the acceptable range of 90.0-110.0%. The placebo was confirmed for the absence of the drug.

TABLE 45
Results for Assay of Diclofenac

| Batch | Initial | Post Study |
|---|---|---|
| 178004-2206 | 99.8% | 98.7% |
| 178004-2209 | 98.3% | 105.2% |
| 178004-2210P | Conforms | NT |

Toxicology batches were evaluated for assay of benzyl alcohol as described above. These data (Table 46) were within the acceptable range of 90.0-110.0% with no significant change between the initial and post study evaluations.

TABLE 46
Results for Assay of Benzyl Alcohol

| Batch | Initial | Post Study |
|---|---|---|
| 178004-2206 | 98.8 | 97.0 |
| 178004-2209 | 100.2 | 98.9 |
| 178004-2210P | 102.5 | NT |

Toxicology batches were evaluated for related compounds/impurities as described above. These data (Table 47) evidenced no significant change between the initial and post study analysis.

TABLE 47
Results for Related Compounds

| Batch | Initial | Post Study |
|---|---|---|
| 178004-2206 | Benzaldehyde: 0.47% | Benzaldehyde: 0.65% |
| | UNK Imp: ND | UNK Imp: <LOQ |
| | Total % Imp: 0.47% | Total % Imp: 0.65% |
| 178004-2209 | Benzaldehyde: 0.47% | Benzaldehyde: 0.70% |
| | UNK RRT 0.70: 0.11% | UNK RRT 1.39: <LOQ |
| | Total % Imp: 0.58% | Total % Imp: 0.70% |
| 178004-2210P | ND | NT |

pH and appearance were evaluated for the three test batches using a calibrated pH meter. The pH was measured by diluting approximately 1.0 g of gel with 10.0 g of carbon dioxide free water. The results for pH (Table 48) and appearance (Table 49) evidenced no significant change between the initial and post study evaluation.

TABLE 48
Results for Related Compounds

| Batch | Initial | Post Study |
|---|---|---|
| 178004-2206 | 4.6 | 4.6 |
| 178004-2209 | 4.6 | 4.5 |
| 178004-2210P | 4.6 | NT |

TABLE 49
Appearance Results

| | Initial Appearance | | | Post Study Appearance | | |
|---|---|---|---|---|---|---|
| | | Absence | Absence of | | Absence | Absence of |
| | | of | Agglomerates/ | | of | Agglomerates/ |
| | | Phase | Particulate | | Phase | Particulate |
| Batch No. | Color | Separation | Matter | Color | Separation | Matter |
| 178004-2206 | White | Conforms | Conforms | White | Conforms | Conforms |
| 178004-2209 | White | Conforms | Conforms | White | Conforms | Conforms |
| 178004-2210P | Clear | Conforms | Conforms | NT | NT | NT |

These data show there was no significant change between the initial and post study evaluations of the compositions.

### Example 14: In Vitro Vaginal Mucosa Permeation and Penetration Testing of Diclofenac Formulations

Three strengths of diclofenac formulations (DARE-PDM1 at 1%, 3%, and 5%) were evaluated for penetration and permeation in reconstituted vaginal epithelia (RVE) constructs.

The diclofenac formulations are evaluated for treatment of pain associated with menstruation in the absence of pelvic pathology (primary dysmenorrhea). The aim of this study is to perform in vitro permeation testing of diclofenac formulations when applied to reconstructed vaginal epithelium. A summary of the formulations evaluated in the Study Plan is in Table 50.

TABLE 50
Summary of formulations

| | | | API | | |
|---|---|---|---|---|---|
| | Figure | Active | Concentration | | |
| Name | Label | Compound | {% w/v} | Lot/Batch | Expiration |
| 1% | 1% | Diclofenac | 1% | 178004-2201 | Mar. 15, 2023 |
| Diclofenac | Diclofenac | | | | |
| Vaginal Gel | | | | | |
| 3% | 3% | Diclofenac | 3% | 178004-2209 | Jun. 30, 2023 |
| Diclofenac | Diclofenac | | | | |
| Vaginal Gel | | | | | |
| 5% | 5% | Diclofenac | 5% | 178004-2211 | Jul. 14, 2023 |
| Diclofenac | Diclofenac | | | | |
| Vaginal Gel | | | | | |

Reconstructed vaginal epithelial (RVE) was cultured per the manufacturer's instructions. This tissue model exhibits in vivo-like morphological and functional characteristics. Dulbecco's Modified Eagle Medium (DMEM) was used as a receptor solution in which diclofenac is stable with and without the presence of vaginal tissue construct. 80% acetonitrile was used as the extraction fluid in which diclofenac can be recovered from the tissue.

A small-scale in vitro permeation study was performed using translucent polycarbonate

transwell® permeable to determine time points for calculating the release rate of diclofenac to be evaluated in the full-scale study. Diclofenac vaginal gel formulation (3%) was used during the small-scale permeation investigation (n=2 per active [diclofenac], n=1 per control). Experimental conditions for the small-scale and full-scale studies are shown in Table 51.

TABLE 51
Experimental conditions for the in vitro permeation studies

| Group | Small Scale | Full Scale |
|---|---|---|
| Tissue type | Reconstructed | Reconstructed |
| | Vaginal Epithelium | Vaginal Epithelium |
| Receptor solution | DMEM | DMEM |
| No. formulations | 1 | 3 |
| No. replicates | 2 | 5 |
| No. controls | 1 | 2 |
| Dose amount | 19 μL/well (0.6 cm²) | 19 μl/well (0.6 cm²) |
| Sampling Volume | 1 mL | 1 mL |
| TEER measurement | 3 hr prior to dosing | 3 hr prior to dosing |
| RS collection times | 1, 2, 4, 6, 8, 21, 22, 24 h | 1, 2, 4, 6, 8, 21, 22, 24 h |

### Study Procedure:

In the small-scale study, a single diclofenac formulation (3%) was analyzed at 8 time points (e.g., 1, 2, 4, 6, 8, 21, 22, 24 h) to observe the amount of diclofenac permeated through RVE into the receptor solution.

Each formulation was applied to the surface of the reconstructed vaginal epithelium (RVE) tissue described above. The permeation of diclofenac was measured by monitoring the rate of appearance of diclofenac in the receptor solution sampled underneath the tissue. Translucent polycarbonate Transwell® permeable inserts with an average surface area of approximately 0.33 cm² and a volume of 0.5 mL were used. Approximately 3 hours prior to dosing, a Transepithelial/transendothelial electrical resistance (TEER) measurement was taken from each RVE tissue sample. The basolateral side was filled with fresh DMEM and the diclofenac formulation (3%) was applied to the apical side. The contents of the basolateral side were collected at each time point and replaced with fresh media. At the end of the study (following the 24 h time point), RVE samples were harvested and diclofenac extracted by lysis in two test extraction fluids (e.g., methanol or 80% acetonitrile).

In the full-scale study, a reconstructed vaginal epithelium permeation and penetration study was performed to compare three diclofenac gel formulations (1%, 3%, 5%) with n=5 per formulation. Diclofenac in the tissue was extracted from the RVE sample following the last time point as described above.

The cumulative amount of diclofenac per cm² that permeated into the receptor solution will be calculated from diclofenac concentrations measured in the receptor solution at each time point. The cumulative amount of diclofenac per cm² at the final time point was calculated for each replicate. Means and standard deviations were calculated and reported in figures and tables described herein. The data was log transformed, and the mean responses from the formulations were statistically compared. Flux rates, where possible, were calculated from the diclofenac concentrations measured in the receptor solution. Flux data, where possible, are represented as μg/cm²/hr. The data was log transformed, and the mean responses from the formulations were statistically compared.

### Results: Small-Scale Study

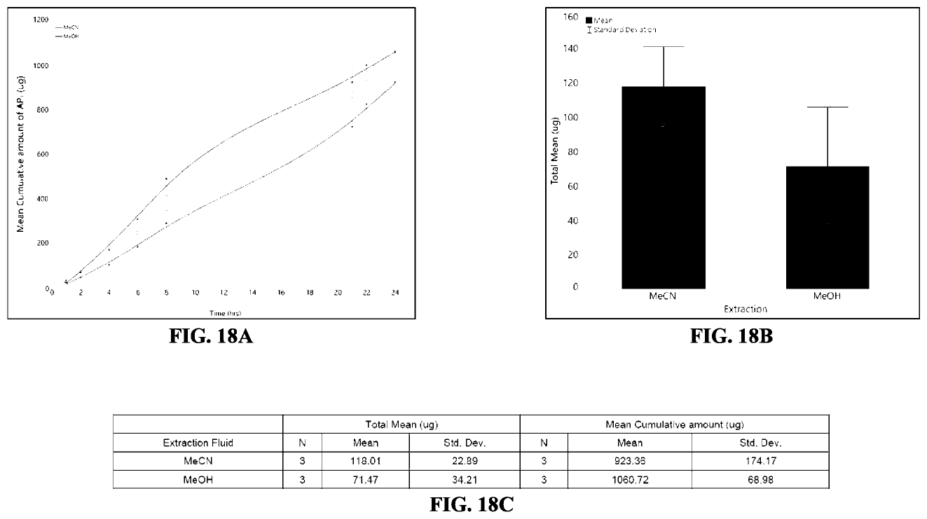

The mean cumulative amount (μg) of diclofenac which permeated into the receptor solution (DMEM) over 24 hours following the application of 3% diclofenac vaginal gel and extracted by MeCN and MeOH is shown in FIG. 18A and described in FIG. 18C. The total mean (μg) of diclofenac extracted by MeCN and MeOH is shown in FIG. 18B.

### Results: Full-Scale Study

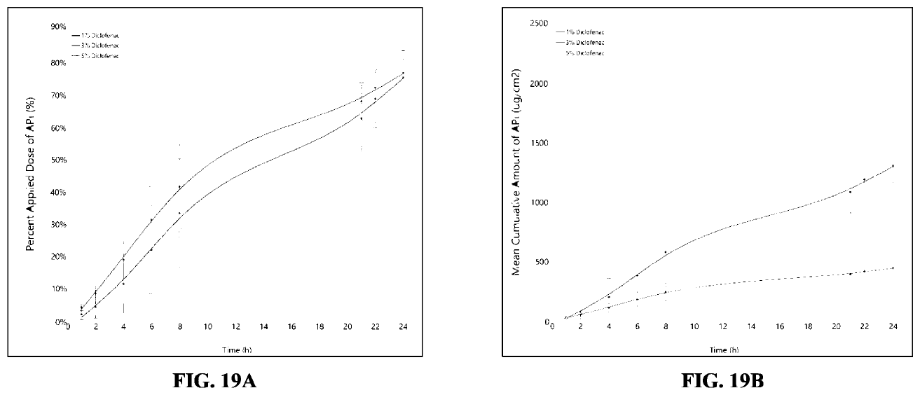

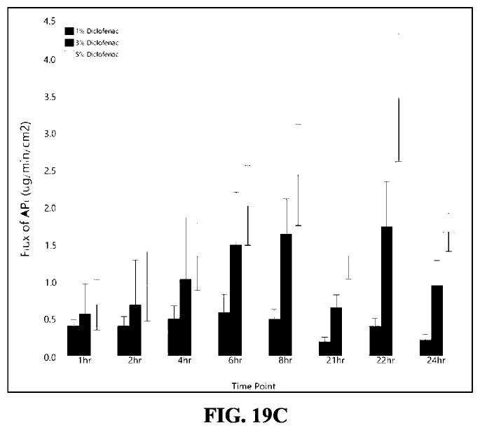

Three diclofenac formulations (1%, 3%, 5%) were evaluated at n=5, and receptor solution was collected at 1, 2, 4, 6, 8, 21, 22, and 24 hours post-dosing. At the 24 h time point, the formulations delivered a total of 0.44, 1.31, and 2.14 mg/cm² diclofenac (FIG. 19B) through the epithelium for the 1%, 3%, and 5% formulations, respectively. These differences were significant with p<0.0001 for each comparison evaluated. The total amount permeated correlated closely with diclofenac concentration in the formulation, resulting in consistent applied dose permeated for each formulation: 76.9%, 75.6%, and 74.4% for 1%, 3%, and 5% formulations, respectively). A biphasic flux pattern was observed, with each formulation showing two peaks in flux at approximately 6-8 h and approximately 22 h post-dosing. The percent applied dose (%) is shown in FIG. 19A, mean cumulative amount (μg/cm²) is shown in FIG. 19B, and flux (μg/min/cm²) of diclofenac is shown in FIG. 19C. Table 52 shows the percent applied dose of diclofenac (%) delivered to the receptor solution at 24 h post application of the 3 formulations. Table 53 shows the mean cumulative amount of diclofenac (μg/cm²) delivered to the receptor solution at 24 h post application of the 3 formulations. Table 54 shows the flux of diclofenac (μg/cm²) delivered to the receptor solution at each time point post-application of the 3 formulations.

TABLE 52
Percent applied dose of diclofenac (%)

| | time | Percent Applied Dose (%) | | |
|---|---|---|---|---|
| Formulation | (h) | N | Mean | Std Dev |
| 1% Diclofenac | 1 | 6 | 3.52% | 1.91% |
| | 2 | 6 | 7.07% | 3.94% |
| | 4 | 6 | 15.82% | 9.23% |
| | 6 | 6 | 26.10% | 15.85% |
| | 8 | 6 | 34.71% | 20.61% |
| | 21 | 6 | 56.80% | 28.29% |
| | 22 | 6 | 60.28% | 29.86% |
| | 24 | 6 | 64.09% | 31.63% |
| 3% Diclofenac | 1 | 6 | 1.63% | 1.53% |
| | 2 | 6 | 3.63% | 3.63% |
| | 4 | 6 | 9.64% | 9.44% |
| | 6 | 6 | 18.33% | 15.23% |
| | 8 | 6 | 27.86% | 20.29% |
| | 21 | 6 | 52.39% | 27.25% |
| | 22 | 6 | 57.45% | 29.27% |
| | 24 | 6 | 62.97% | 31.74% |
| 5% Diclofenac | 1 | 6 | 1.20% | 0.86% |
| | 2 | 6 | 2.84% | 2.06% |
| | 4 | 6 | 7.50% | 4.85% |
| | 6 | 6 | 14.55% | 8.55% |
| | 8 | 6 | 23.04% | 13.10% |
| | 21 | 6 | 50.12% | 25.09% |
| | 22 | 6 | 56.17% | 27.95% |
| | 24 | 6 | 61.98% | 30.78% |

TABLE 53
Mean cumulative amount of diclofenac (ug/cm²)

| | Mean Cumulative Amount | | |
|---|---|---|---|
| | (ug/cm2) | | |
| Formulation | N | Mean | Std Dev |
| 1% Diclofenac | 5 | 442.83 | 24.35 |
| 3% Diclofenac | 5 | 1305.27 | 143.85 |
| 5% Diclofenac | 5 | 2141.28 | 161.40 |

TABLE 54
Flux of diclofenac (ug/cm²)

| | | Flux (ug/min/cm2) | | |
|---|---|---|---|---|
| Formulation | TP | N | Mean | Std Dev |
| 1% Diclofenac | 1 hr | 5 | 0.41 | 0.09 |
| | 2 hr | 5 | 0.41 | 0.12 |
| | 4 hr | 5 | 0.50 | 0.17 |
| | 6 hr | 5 | 0.59 | 0.24 |
| | 8 hr | 5 | 0.50 | 0.14 |
| | 21 hr | 5 | 0.20 | 0.06 |
| | 22 hr | 5 | 0.40 | 0.11 |
| | 24 hr | 5 | 0.22 | 0.07 |
| 3% Diclofenac | 1 hr | 5 | 0.56 | 0.42 |
| | 2 hr | 5 | 0.69 | 0.61 |
| | 4 hr | 5 | 1.04 | 0.82 |
| | 6 hr | 5 | 1.50 | 0.71 |
| | 8 hr | 5 | 1.65 | 0.47 |
| | 21 hr | 5 | 0.65 | 0.18 |
| | 22 hr | 5 | 1.75 | 0.61 |
| | 24 hr | 5 | 0.95 | 0.34 |
| 5% Diclofenac | 1 hr | 5 | 0.69 | 0.34 |
| | 2 hr | 5 | 0.95 | 0.47 |
| | 4 hr | 5 | 1.34 | 0.46 |
| | 6 hr | 5 | 2.03 | 0.53 |
| | 8 hr | 5 | 2.45 | 0.68 |
| | 21 hr | 5 | 1.20 | 0.16 |
| | 22 hr | 5 | 3.48 | 0.86 |
| | 24 hr | 5 | 1.67 | 0.26 |

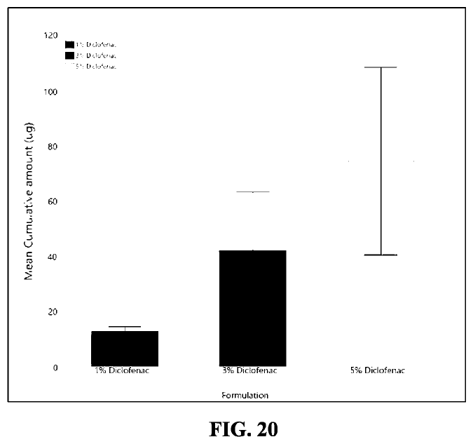

To evaluate diclofenac tissue penetration, the mean cumulative amount of diclofenac (μg) present in the reconstructed vaginal tissues is shown in FIG. 20 and Table 55. Total amount of drug in tissue after 24 h of treatment also measured: 12.81, 42.15, and 74.64 μg total diclofenac was extracted from tissues treated with the 1%, 3%, or 5% formulations, respectively.

TABLE 55
Mean cumulative amount of diclofenac (μg) delivered
to the reconstructed vaginal tissues after 24 h.

| | Mean Cumulative amount | | |
|---|---|---|---|
| | (ng) | | |
| Formulation | N | Mean | Std Dev |
| 1% Diclofenac | 4 | 12.80 | 1.82 |
| 3% Diclofenac | 5 | 42.15 | 21.19 |
| 5% Diclofenac | 5 | 74.64 | 34.13 |

### CONCLUSIONS

Three strengths of diclofenac formulations (1%, 3%, and 5%) were evaluated for penetration and permeation in reconstituted vaginal epithelia (RVE) constructs. A small-scale study was conducted over a 24 h period to assess time points and extraction methods using the 3% formulation at n=3. Acetonitrile was selected as the extraction fluid, and the same 24 h time course was selected for the full-scale study.

All three formulations were tested at n=5, and receptor solution was collected at 1, 2, 4, 6, 8, 21, 22, and 24 hours post-dosing. At the 24 h time point, the formulations delivered a total of 0.44, 1.31, and 2.14 mg/cm² through the epithelium for the 1%, 3%, and 5% formulations, respectively. These differences were significant at p<0.0001 for each comparison. A biphasic flux pattern was observed, with each formulation showing two peaks in flux at approximately 6-8 h and at approximately 22 h. Total amount of diclofenac in tissue after 24 h of treatment was also measured, 12.81, 42.15, 74.64 μg total diclofenac was extracted from the tissues treated with the 1%, 3%, or 5% formulations, respectively.

### Example 15: Diclofenac: Vaginal Irritation Study in Rabbits

The objective of the study was to determine the potential vaginal irritation of diclofenac, when administered intravaginally to female rabbits for 7 Days and to assess the reversibility, persistence, or delayed occurrence of toxic effects following a 14-Day recovery period.

In this study, fifteen female rabbits (New Zealand White) were randomly assigned to 3 groups of 5 rabbits/group (3 rabbits in dosing phase and 2 rabbits in recovery phase). Animals were given Vaginal Gel Placebo, 3%, and 10% Diclofenac Vaginal Gel at a dose volume of 1 mL/animal intravaginally once daily for 7 days. At the initiation of dosing, animals were approximately 3 months old and with body weights ranging from 2.5 to 2.9 kg. Females were nulliparous and non-pregnant.

Criteria for evaluation included mortality, clinical observations, scoring of vaginal irritation reactions, body weight, clinical pathology (serum chemistry), gross pathology and histopathological evaluation. There were no unscheduled deaths during the course of study. There were no test article (diclofenac)-related changes to clinical signs, body weight, or Creatine Kinase (CK) observed in this study.

There were no macroscopic findings in the vagina at the scheduled termination. The test article was considered a non-irritant based on the histopathology results and the Irritation Index. Intravaginal administration with Vaginal Gel Placebo, 3%, and 10% Diclofenac Vaginal Gel once daily for 7 days to the rabbit at dose volume of 1 mL/animal following a 14-Day recovery period was well tolerated. There was no evidence of any test article related vaginal irritation observed at each dose level.

### Study Design

Fifteen female rabbits were randomly assigned to 3 groups of 5 per group to determine the vaginal irritation of diclofenac vaginal gel (DARE-PDM1) when administered intravaginally once daily for 7 days. The control group was administered control (placebo) formulation. Animals were randomly assigned to groups based on body weight. 2 rabbits per group were allocated for recovery. The study design is summarized in Table 56.

### Administration

The test article (diclofenac) was administered intravaginally. The test or control formulation were instilled in the vaginal vault of the rabbits. The syringe was filled with the test or control formulation with one syringe prepared for each animal. The head of the syringe was inserted slowly and gently (approximately 4 cm deep). The dosage volume was 1.0 mL/animal. Then the animal was put back in the cage. After vaginal administration, Elizabeth collars were used for 4 hours after dosing to prevent the rabbits from ingesting gel that might leak from the vagina. The acclimation to Elizabeth collars were conducted prior to the start of treatment. The acclimation time was respectively set as initially 30 min then moving up to 1 h, 3 h, and 5 h.

TABLE 56
Study Design

| | | DiclofenacDoses ^{a} | | | | Numbering of Animals | |
|---|---|---|---|---|---|---|---|
| Group | Group | Dose | Volume (mL/ | Conc. (mg/ | Conc. | Dosing Phase | Recovery |
| Numbers | Designation | (mg day) | animal) | mL) | (w/w) | Female | Female |
| 1 | Control | 0 | 1.0 | 0 | 0 | 1501-1503 | 1504-1505 |
| 2 | Low Dose | 30 | 1.0 | 30 | 3% | 2501-2503 | 2504-2505 |
| 3 | High Dose | 100 | 1.0 | 100 | 10% | 3501-3503 | 3504-3505 |

Note:
In this report “dose level” and “dosage” are used interchangeably.
^{a} Doses represent active ingredient.
Conc. = Concentration

### Results

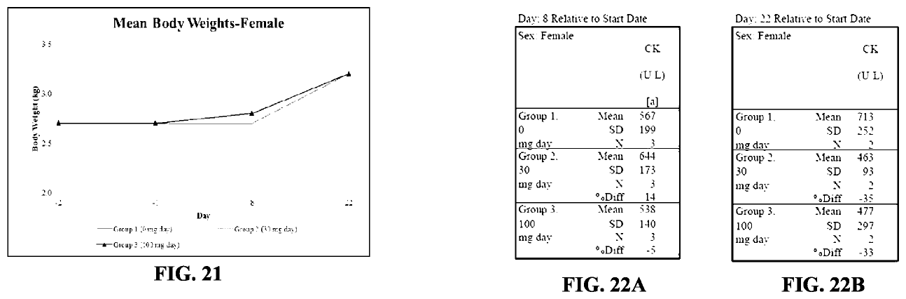

There were no unscheduled deaths in this study. All study animals survived to the scheduled necropsy dates. There was no test article (diclofenac) related vaginal irritation observed in the study. There were no test article related changes on body weight (FIG. 21). The variations in body weight observed were consistent with those seen in untreated rabbits. There was no test article related changes in serum Creatine Kinase (CK) observed in the study (FIG. 22A and FIG. 22B), the variations in CK observed were consistent with those seen in untreated rabbits. There were no test article-related macroscopic changes to examined tissues in any treatment groups at the end of the dosing and recovery phases.

In the microscopic evaluations of histopathology, after subtracting the control group average score, the Irritation Index in group 2 (3% diclofenac) and group 3 (10% diclofenac) were −0.34 or −0.45, respectively in the dosing phase. The test article was considered non-irritant based on the histopathology results and the Irritation Index, shown in Table 57.

TABLE 57
Irritation Score in the Dosing Phase

| Group | 1 | | | 2 | | | 3 | | |
|---|---|---|---|---|---|---|---|---|---|
| Sex | Female | | | Female | | | Fernale | | |
| Animal | 1501 | 1502 | 1503 | 2501 | 2502 | 2503 | 3501 | 3502 | 3503 |
| No. | | | | | | | | | |
| Total | 3 | 3 | 1 | 0 | 1 | 3 | 2 | 0 | 1 |
| Score | | | | | | | | | |

| | | | |
|---|---|---|---|
| Group | 7 | 4 | 3 |
| Total | | | |
| Score | | | |
| Group | 0.78 (7/9) | 0.44 (4/9) | 0.33 (3/9) |
| Average | | | |
| Score | | | |
| Irritation | NA | −0.34 | −0.45 |
| Index | | | |

In the microscopic evaluations of histopathology, after subtracting the control group average score, the Irritation Index in group 2 (3% diclofenac) and group 3 (10% diclofenac) were 0.00 or −0.17, respectively in the recovery phase. The test article was considered non-irritant based on the histopathology results and the Irritation Index, shown in Table 58.

TABLE 58
Irritation Score in the Recovery Phase

| Group | 1 | | 2 | | 3 | |
|---|---|---|---|---|---|---|
| Sex | Female | | Female | | Female | |
| Animal No. | 1504 | 1505 | 2504 | 2505 | 3504 | 3505 |
| Total Score | 0 | 1 | 1 | 0 | 0 | 0 |

| | | | |
|---|---|---|---|
| Group Total Score | 1 | 1 | 0 |
| Group Average Score | 0.17 (1/6) | 0.17(1/6) | 0(0/6) |
| Irritation Index | NA | 0.00 | −0.17 |

In conclusion, intravaginal administration with Vaginal Gel Placebo, 3%, and 10% Diclofenac Vaginal Gel once daily for 7 days to the rabbit at dose volume of 1 mL/animal following a 14-Day recovery period was well tolerated. There was no evidence of any test article (diclofenac) related vaginal irritation observed at any dose level.

### Example 15: a Phase 1, Multi-Site, Randomized, Placebo-Controlled, Double-Blind Study to Evaluate the Pharmacokinetics, Safety and Preliminary Efficacy of Two Strengths of Dare-PDM1 (1% Diclofenac or 3% Diclofenac) Versus Placebo Among Women with Symptomatic Primary Dysmenorrhea

This Phase 1 study aims to evaluate the in vivo pharmacokinetics (PK) in the systemic (plasma) and genital (vaginal fluid) compartments, after a single dose or 3 daily doses of either 1% or 3% diclofenac in 2.5 mL of DARE-PDM1 hydrogel or placebo hydrogel for the treatment of primary dysmenorrhea. Preliminary efficacy (changes during 7-day observation windows in daily DAP scores at the pelvic/vaginal and low back anatomic sites) will be assessed. Preliminary efficacy will also be measured by recording the use of study provided rescue oral paracetamol or other non-pharmacologic pain-relieving measures. The study will be conducted among healthy premenopausal women with primary dysmenorrhea who are not taking hormonal contraception.

### Primary Objectives of the Study:

Pharmacokinetics (PK): To evaluate the systemic and local vaginal fluid genital PK of diclofenac after a single dose and multiple (three) doses of placebo or DARE-PDM1 (in two different strengths, 1% or 3% diclofenac in 2.5 mL hydrogel).
Safety: To evaluate genital and systemic safety of DARE-PDM1 (in two different strengths, 1% or 3% diclofenac in 2.5 mL of hydrogel) versus placebo given as a single dose or multiple doses.

### Exploratory Objective of the Study:

Preliminary Efficacy: To evaluate the preliminary efficacy of DARE-PDM1, given in two different strengths (1% or 3% diclofenac in 2.5 mL of hydrogel), versus placebo, compared to a baseline, no treatment cycle, in reducing dysmenorrhea associated pain (DAP) from primary dysmenorrhea.
Use of rescue pain medications or alternative pain relief methods: To evaluate the number of rescue (study-supplied) oral pain medication doses (paracetamol) or other pain-relieving modalities (e.g., heating pad) needed post gel dosing by participants in all treatment groups (placebo versus DARE-PDM1 1% Strength versus DARE-PDM1 3% Strength) compared to a baseline, control, no treatment menstrual cycle.

### Primary Endpoints:

Systemic and Vaginal Pharmacokinetics (PK): Plasma and vaginal fluid concentrations of diclofenac will be measured in the DARE-PDM1 1% Strength and DARE-PDM1 3% Strength and Placebo groups to describe the following:

### Plasma

Single dose: PK parameters for diclofenac in plasma (Cmax, C24, tₘₐₓ, AUC0-24, AUClast and t½, eff) following a single vaginal dose administration (PK sampling up to 120 hr)
Single dose: Blood will be obtained for plasma diclofenac concentrations prior to dosing (to confirm negative) and at 0.5, 1, 2, 4, 8 and 12 (±15 minutes) post single dose. Participants will return to the CRU for visits 4-8 at 24, 48, 72, 96 and 120 hours (±4 hours) respectively, after the single dose administration for plasma PK sampling.

### Vaginal Fluid

Single dose: Vaginal fluid concentrations of diclofenac following a single vaginal dose administration (PK sampling up to 120 hr)
Single dose: Vaginal fluid for diclofenac concentration will be obtained, using a blind vaginal swab, prior to dosing (to confirm negative) and at 1, 2, 4 and 8 hours (±15 minutes) post single dose administration. Participants will return to the CRU for visits 4-8 at 24, 48, 72, 96 and 120 hours (±4 hours) respectively, after the single dose administration for vaginal fluid sampling.
Multiple dose: Trough vaginal fluid concentration of diclofenac 24 hours after the third and final dose of the 3-day multi-dose (MD) regimen.

### Duration of Study

Participant participation is expected to last up to 2-3 months, including a screening visit, enrollment visit, baseline menstrual cycle and post single dose and multiple dose treatment assessments.

### Safety:

Comparisons will be made from pre-dosing baseline to during treatment for each participant. Safety will be assessed by adverse events (AEs), collected from enrollment at visit 2 until the end of the study at visit 9. AEs occurring prior to and after dosing will be described and AEs will be compared between the three study drug groups. Relatedness of the AE to study products or study procedures will also be categorized and described per treatment group. Comparisons will also be made between the three independent product groups (Placebo, DARE-PDM1 1% strength, and DARE-PDM1 3% strength) in the following endpoints:
Treatment emergent adverse events (TEAEs) graded using the National Institutes of Health (NIH) Division of AIDS (DAIDS) Table for Grading the Severity of Adult and Pediatric Adverse Events (July 2017). Genitourinary symptoms graded using the NIH DAIDS Addendum 1, Female Genital Grading Table for Use in Microbicide Studies (September 2017).
Changes from baseline in screening safety laboratories.
Visualization of the cervico-vaginal (CV) mucosa on speculum exam

### Exploratory Endpoints:

### Preliminary Efficacy:

Dysmenorrhea associated pain (DAP) intensity will be assessed daily on an 11-point (0-10) scale by the following question: Rate the pain in the anatomical area (low back pain, pelvic/vaginal pain), during the past 24 hours, where 0=no pain and 10=worst imaginable pain. Participants will record daily DAP scores (scale 0-10) for two anatomic sites: low back pain and pelvic/vaginal pain. Two 7-day assessment windows, with day 1 being the first day that the participant records an increase in her DAP score over her a baseline at either the pelvic/vaginal location or low back location, will be compared, one during the baseline, no treatment, control, 7-day assessment period and a second during the post multiple dose treatment 7-day assessment period. During these 7-day assessment windows, the maximum DAP score, the cumulative 7-day DAP score, and the average daily DAP score will be calculated for both of the anatomic locations (pelvic/vaginal pain and low back pain). These DAP score indices will be compared between the baseline, no treatment, control 7-day assessment period and the post multiple dose treatment 7-day assessment period. Comparisons of DAP scores (pelvic/vaginal pain and low back pain) will also be made between the three independent study product groups during the post multiple dose treatment 7-day assessment window.

Participants will commence the multiple dose regimen by inserting their first dose of gel at home on the day when they first experience an increase in dysmenorrhea associated pain. This 7-day window will start when the participant administers the first of three daily gel doses at home. The first dose of the multiple dose regimen will be administered at home by the participant when the participant experiences an increase in dysmenorrhea, above her baseline at either anatomic site. In order to compare pain scores over an equal number of days in the control/baseline/no treatment and post multiple dose post treatment periods, the two 7-day observation periods will be selected and assessed from the daily recorded DAP score data. Because the single gel dose will not be timed according to dysmenorrhea symptoms, the post single dose period will only be used to assess safety and single dose PK (with up to 120 hours PK sampling) and not the exploratory endpoint of preliminary treatment efficacy.

Participants will commence reporting daily DAP scores for the low back and pelvic/vagina anatomic sites on the day after enrollment (visit 2). The study coordinator will counsel the patient to specifically focus on dysmenorrhea associated pain for this diary entry. Although pain scores will be collected daily, pain scores reported during two 7-day observation periods will be defined, as above, in the (1) control, baseline, no treatment observation period and (2) the post multiple dose treatment observation period. The 7-day observation periods for the control, baseline, no treatment cycle and the post multiple dose treatment period are timed to begin when the participant records an increase in her DAP above her baseline at either anatomic site.

For each of the two 7-day observation periods, the maximum DAP score during the 7 days, a cumulative 7-day DAP score, and an average DAP score over the 7 days, for each anatomic area (pelvic/vaginal pain and low back pain) will be reported and compared between the baseline cycle and the multiple dose treatment cycle.

### Use of Rescue Pain Medication or Alternative Pain Relieving Methods:

The number of doses of study-provided oral paracetamol and or other non-pharmaceutical pain-relieving measures (e.g., use of heating pad) required during the post multiple dose treatment 7-day assessment windows (as above) will be compared using independent group statistics, for the three treatment groups (1%, 3%, placebo) during the post multiple dose treatment 7-day observation period.

### Study Design:

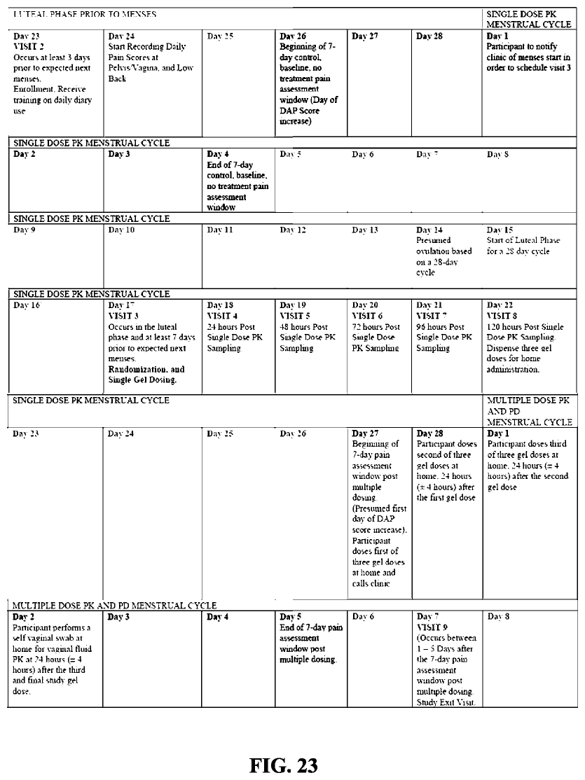

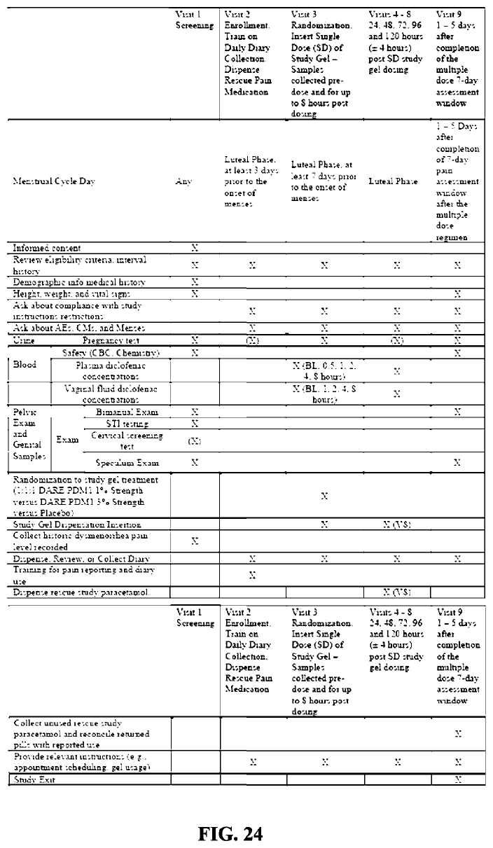

This is a Phase 1, multi-site, randomized, placebo-controlled, double-blind, 3 arm parallel group study of approximately 36 healthy, premenopausal women with primary dysmenorrhea. This study is designed to assess the systemic (plasma) and local mucosal (vaginal fluid) diclofenac PK and safety after a single dose and during three daily doses of vaginally administered DARE-PDM1, given in two different strengths (1% or 3% diclofenac in 2.5 mL of hydrogel) versus placebo. The study will also assess, as an exploratory endpoint, the preliminary dysmenorrhea treatment efficacy of DARE-PDM1, given in two different strengths (1% or 3% diclofenac in 2.5 mL of hydrogel) versus placebo, when dosed in three daily doses at the onset of dysmenorrhea symptoms, compared to a no-treatment, baseline, control cycle. The study observation period will encompass approximately three menstrual cycles (detailed in FIG. 23 and FIG. 24).

Randomization will occur at visit 3 prior to initiation of treatment and participants will be randomized in a 1:1:1 ratio to the treatment groups below (Table 58). DARE-PDM1 is administered as a vaginal hydrogel, 2.5 mL volume. This study will investigate a single dose, administered in the clinic and a 3-day multiple dose regimen, administered at home. Participants will therefore receive 4 total doses. The single dose will be self-administered in the clinic at visit 3. The three doses of the multiple dose regimen will be administered by the participant at home. The participant will be instructed to remain recumbent for 15 minutes after dosing of the gel.

TABLE 58
Treatment Groups

| Treatment Group and Number | Treatment |
|---|---|
| DARE-PDM1 1% Diclofenac | 4 total doses: 1 single dose and |
| in 2.5 mL Hydrogel (n = 12) | 3 multiple doses (once daily) |
| DARE-PDM1 3% Diclofenac | 4 total doses: 1 single dose and |
| in 2.5 mL Hydrogel (n = 12) | 3 multiple doses (once daily) |
| Placebo 2.5 mL Hydrogel | 4 total doses: 1 single dose and |
| (n = 12) | 3 multiple doses (once daily) |

In order to be enrolled, participants must report, for at least one of two anatomic sites (pelvic/vaginal pain or low back pain), past, historic, dysmenorrhea associated pain levels of ≥5 on a scale of 0-10, where 0 is no pain and 10 is the worst imaginable pain. They cannot have a chronic pain syndrome other than dysmenorrhea which could confound preliminary efficacy data (e.g. chronic low back pain unrelated to menses). The volunteer will report her average level of dysmenorrhea associated pain in the past, specifically the amount of pain she has when she is not using treatments for dysmenorrhea, which include NSAIDS and hormonal contraception. It will also be noted at baseline, whether the participant reports that dysmenorrhea significantly disrupts her life (having to miss school, miss work, or modify her daily activities due to dysmenorrhea pain on at least two occasions in the past).

During the study, the participant will record daily dysmenorrhea associated pain (DAP) scores at both the pelvic/vagina and low back locations using the same 11-point numeric rating scale (0-10).

Pharmacokinetics (PK) (primary objective): The in vivo PK profile will be described for both regimens (single dose and multiple dose) in this study. Preliminary dysmenorrhea treatment efficacy (pharmacodynamics, PD) will be an exploratory endpoint and will be assessed during the multiple dose regimen, as this regimen is started based on the onset of dysmenorrhea symptoms.

The following PK parameters for plasma diclofenac will be determined after the single dose administration:
C24h: time-weighted average observed plasma concentration over 24 hours (Day 1 PK profile)
Cmax: maximum observed plasma concentration after a single dose, for up to 120 hours post dosing
tₘₐₓ: the time that Cₘₐₓ was observed
AUC₀₋₂₄: area under the plasma concentration-time curve from time 0 to 24 hours; calculated using the linear/log trapezoid rule
AUC_{LAST}: area under the plasma concentration-time curve from time 0 (Day 1) to the time of the last quantifiable concentration up to 120 hours post single dosing; calculated using the linear/log trapezoid rule
t½_{eff}: effective half-life; calculated using AUC₀₋₂₄

Safety (primary objective) will be assessed by adverse events (AEs), collected from enrollment at visit 2 until the end of the study at visit 9. AEs occurring prior to and after dosing will be described and AEs will be compared between the three study drug groups. Relatedness of the AE to study products or study procedures will also be categorized and described per treatment group. Preliminary efficacy (exploratory objective) of the study product will be measured by reported daily DAP scores. Participants will record daily DAP scores throughout the study. For the exploratory endpoint of preliminary efficacy, DAP scores will be assessed during two 7-day observation periods, one in the control, baseline, no treatment, cycle and the other in the multiple dose cycle.

The participant will be randomized to study gel (1:1:1, DARE-PDM1 1% Strength versus DARE-PDM1 3% Strength versus placebo hydrogel) and will insert the study product in the clinic. The participant will remain recumbent for approximately 15 minutes after study gel dosing. Subjects will then have blood drawn for plasma diclofenac concentrations at 0.5, 1, 2, 4 and 8 hours (±15 minutes) post dosing. Participants will have blind vaginal swabs performed for vaginal fluid diclofenac concentrations at 1, 2, 4 and 8 hours (±15 minutes for each time point) post dosing.

They will continue to record in their daily diary AEs (safety), menstrual bleeding concomitant medications, and daily DAP scores at the 2 anatomic sites (preliminary efficacy). The participant will be instructed to not use tampons and to place nothing in the vagina and to avoid vaginal intercourse over the next 120 hours (until completion of visit 8), so that vaginal fluid PK measurements will not be confounded by the presence of semen. The participant will return to the clinic 24 hours (±4 hours) after the single vaginal dose for visit 4.

TABLE 59
Clinical Laboratory Tests

| Hematology (Complete Blood Count) | Chemistry Metabolic Panel |
|---|---|
| Hematocrit | Albumin |
| Hemoglobin | Alkaline phosphatase |
| Red blood cell count | Alanine aminotransferase |
| White blood cell count | Aspartate aminotransferase |
| Neutrophils (absolute) | Gamma glutamyl transferase |
| Lymphocytes (absolute) | Direct bilirubin |
| Monocytes (absolute) | Total bilirubin |
| Basophils (absolute) | Lactate dehydrogenase |
| Eosinophils (absolute) | Total protein |
| Platelet count (estimate not acceptable) | Blood urea nitrogen |
| | Creatinine |
| | Calcium |
| | Phosphate |
| | Sodium |
| | Potassium |
| | Bicarbonate |
| | Chloride |
| | Glucose |

### Adverse Events (AE)

The intensity of an AE will be graded according to the following definitions:
Grade 1: Mild; the participant experiences awareness of signs or symptoms but these are easily tolerated or managed without specific treatment.
Grade 2: Moderate; the participant experiences discomfort enough to cause interference with usual activity, and/or the condition requires specific treatment.
Grade 3: Severe; the participant is incapacitated with inability to work or do usual activity, and/or the event requires significant treatment measures.
Grade 4: Disabling or with life threatening consequences, urgent intervention indicated
Grade 5: Death

### Visits

Visit 1 (V1) is the screening visit. After written informed consent is obtained, participants will undergo screening procedures, including an assessment of eligibility as well as their past dysmenorrhea symptoms which they previously experienced while not using NSAIDs or hormonal contraception (since both are effective, off label dysmenorrhea treatments). The volunteer will be asked to recall her dysmenorrhea symptoms in the past, over an approximate 3-month period of time, when she was not using NSAIDs or hormonal contraception, at the two anatomic sites (pelvic/vaginal pain and low back pain). The participant will also be queried if her dysmenorrhea symptoms have caused lifestyle disruption on at least two occasions (e.g. missing work, missing school, missing social events or other disruptions to her lifestyle). At the screening visit, the participant's height, weight, and vital signs will be measured. The subject will also have a urine pregnancy test, testing for sexually transmitted infections (STIs), a speculum exam and a bimanual exam, and a cervical screening test, if indicated. Blood will be obtained for a non-fasting complete blood count (CBC) and blood chemistry panel. Upon completion of screening procedures and confirmation of eligibility, eligible participants will proceed to visit 2.

Visit 2 (V2) is the enrollment visit. V2 will occur in the luteal phase of the participant's menstrual cycle and at least 3 days prior to the start of her menses. The main purpose of visit 2 is to commence daily dysmenorrhea associated pain (DAP) diary scores, so that the control, baseline, no treatment, pain observation window can be defined. Given that some women experience dysmenorrhea prior to the onset of menses, visit 2 needs to occur prior to the onset of menses. Eligibility criteria and interval medical history will be reviewed. At V2 and all subsequent visits, the study coordinator will query the participant regarding any interval AEs or use of concomitant medications (CON MEDS), as well as timing of menses. At V2, the participant will be given a study diary which she will use to record daily DAP scores for pelvic/vaginal pain and low back pain, AEs, CON MEDS and other notes. The participant will commence collecting diary data on the day after V2 and will collect diary data daily until study exit. The participant may not use over-the-counter pain relievers or prescribed non-study pain medications. The participant will be told to call the study coordinator when her next menses begins, in order to correctly schedule visit 3.

Visit 3 (V3) is the randomization and single dose product dosing visit. As above, this visit will occur in the luteal phase of the menstrual cycle, at least 7 days prior to the subsequent, post single dose treatment menstrual cycle. PK samples will be collected for up to 120 hours after the single dose and menstrual bleeding needs to be avoided for the vaginal fluid PK samples. In addition, single dose PK with sampling up to 120 hours cannot overlap with the start of the multiple dosing (MD) period (discussed below). If the participant has started her menstrual cycle prior to presenting for visit 3, or if the participant is anticipating dysmenorrhea symptoms to begin within 7 days of visit 3, visit 3 will be re-scheduled. At visit 3, the study coordinator will ask the participant about AEs and CON MEDS and will review the participant's diary entries. The participant will provide urine for a pregnancy test. If the participant requests or the investigator deems this to be the most feasible and safe way to obtain multiple time point blood draws, a catheter may be inserted into the participant's vein by qualified study personnel. Pre-dosing vaginal fluid and plasma for diclofenac concentration will be collected, to assure negative values. The participant will be randomized to study gel (1:1:1, DARE-PDM1 1% Strength versus DARE-PDM1 3% Strength versus placebo hydrogel) and will insert the study product in the clinic.

Visit 4 (V4), Visit 5 (V5), Visit 6 (V6), Visit 7 (V7) and Visit 8 (V8): V4 will occur 24 hours (±4 hours) after the single vaginal dose. V5 will occur 48 hours (±4 hours) after the single vaginal dose. V6 will occur 72 hours (±4 hours) after the single vaginal dose. V7 will occur 96 hours (±4 hours) after the single vaginal dose. Finally, V8 will occur 120 hours (±4 hours) after the single vaginal dose. At all of these visits, the study coordinator will query the participant about treatment emergent adverse events (TEAEs) and CON MEDS and will review the participant's diary entries. At each of these visits, the participant will provide a urine pregnancy test if she has signs or symptoms of pregnancy or at the investigator's discretion. Blood and vaginal fluid (blind vaginal swab) will be taken for plasma and vaginal fluid diclofenac concentrations, respectively. At visit 8, the participant will be given three doses of the study product to which she was randomized, a vaginal swab to obtain a vaginal fluid PK sample at home and a 30-day supply of study rescue pain medication (650 mg oral paracetamol tablets). The participant will call the study coordinator on the day that she administers the first of three doses of gel, in order to schedule visit 9.

Visit 9 (V9) will occur between 1-5 days after the post-multiple dose 7-day pain observation assessment period is completed. This window will allow complete 7-day collection of daily diary data (safety and preliminary efficacy data) for the post multiple dose treatment cycle observation period. The study coordinator will query the participant about TEAEs and CON MEDS and will review the participant's diary entries and collect the participant diary. The study coordinator will also collect any unused study-provided paracetamol and reconcile the number of reported doses with the number of returned, unused doses. The participant's height, weight, and vital signs will be measured. The participant will provide a urine pregnancy test. A speculum exam and bimanual exam will be performed. Blood will be obtained for a non-fasting complete blood count and blood chemistry panel. The participant will exit from the study at V12 unless she has TEAEs which require further follow up.

### Participant Selection Criteria:

Approximately 36 women who meet eligibility criteria will be enrolled in the study.

### Inclusion Criteria:

1. Females ages 18-50 years old (inclusive)
2. Self-assessment of historic dysmenorrhea associated pain level of ≥5 on a scale of 0-10 in at least one of the following anatomic sites: pelvic/vaginal pain, low back pain, while not using NSAIDs or hormonal contraception.
3. Non-pregnant status
4. If applicable, agrees to be sexually abstinent and place nothing in the vagina during the 120 hours between visits 3 and 8 and the 3-day period of the multiple dose regimen.
5. Agrees to use adequate non-hormonal birth control during the trial (e.g. study provided male condoms without nonoxynol-9 lubricant, tubal sterilization, heterosexual abstinence)
6. Because hormonal birth control is a known off label treatment for dysmenorrhea, if the participant is on hormonal birth control other than Depo-Provera contraceptive injection, she agrees to discontinue it and have at least one spontaneous intervening menses before the start of the study period. If using Depo Provera, has not had an injection within the 4 months before Visit 1 and must have had a spontaneous menses prior to visit 2.
7. Provides informed consent for participating in the trial
8. Willingness to use only study-provided oral paracetamol as rescue pain medication for dysmenorrhea, if needed according to investigator's instruction.
9. Patient is fluent in the English language.
10. Patient is capable of understanding and complying with the protocol and agrees to sign the informed consent document.
11. Patient has had a cervical screen performed within five years prior to Visit 1 and can provide documentation indicating normal test results consistent with health guidelines. If the patient cannot provide documentation, a cervical screen will be performed at Visit 1. Patients with abnormal findings will be excluded from study participation and be referred for follow-up medical care as appropriate.

### Exclusion Criteria:

1. Positive pregnancy test
2. Unwilling or unable to comply with protocol
3. Allergic to diclofenac or other non-steroidal anti-inflammatory drugs (NSAIDs)
4. Patients with severe liver, kidney or heart failure
5. After the use of aspirin or other nonsteroidal anti-inflammatory drugs, asthma, nasal polyps, angioedema and urticaria have occurred in the past
6. Current active peptic ulcer bleeding or perforation
7. Have a history of significant upper gastrointestinal disease
8. Have a chronic pain syndrome other than dysmenorrhea which could confound preliminary efficacy data (e.g., chronic low back pain unrelated to menses)

Participants are free to discontinue the study at any time, for any reason, and without prejudice to further treatment. The investigator may remove a participant if, in the investigator's judgment, continued participation would pose unacceptable risk to the participant or to the integrity of the study data.

### Definitions

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

“Solution” according to the current disclosure is a clear, homogeneous liquid dosage form that contains one or more chemical substances dissolved in a solvent or mixture of mutually miscible solvents. A solution is a liquid preparation that contains one or more dissolved chemical substances in a suitable solvent or mixture of mutually miscible solvents. Because molecules of a drug substance in solution are uniformly dispersed, the use of solutions as dosage forms generally provides assurance of uniform dosage upon administration and good accuracy when the solution is diluted or otherwise mixed.

“Suspension” as used herein is a liquid dosage form that contains solid particles dispersed in a liquid vehicle.

When ranges are given by specifying the lower end of a range separately from the upper end of the range, it will be understood that the range can be defined by selectively combining any one of the lower end variables with any one of the upper end variables that is mathematically possible. Where ranges are recited, it will be understood that any subrange or value within the recited ranges, including endpoints, is contemplated.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### LIST OF ABBREVIATIONS

ABS: Absolute
ACN: Acetonitrile
ADI: Acceptable Daily Intake
ANOVA: Analysis of Variance
API: Active Pharmaceutical Ingredient
Cat #: Catalog Number
CI: Confidence Interval
CQA: Critical Quality Attribute
Diff: Difference
DOE: Design of Experiment
Drug Product: Diclofenac Vaginal Gel
FA: Formic Acid
G′: Storage Modulus
G″: Loss Modulus
HCl: Hydrochloric Acid
HPLC: High Pressure Liquid Chromatography
HSM: High Shear Mixing
ID: Identification
IIG: Inactive Ingredient
Imp: Impurity
IVRT: In Vitro Release Testing
LC: Label Claim
LOQ: Limit of Quantitation
LVER: Linear Viscoelastic Region
mPa*s: Millipascal seconds [mPa*s are equivalent to centipoise (cP)]
MCR: Modular Compact Rheometer
MeOH: Methanol
NaOH: Sodium Hydroxide
NLT: No Less Than
NMT: No More Than
NF: National Formulary
P/N: Part Number
Pa: Pascals
PBS: Phosphate Buffered Saline
PES: Polyether Sulfone
PN: Part Number
PP25: Parallel Plate 25 (mm)
PSD: Particle Size Distribution
PTFE: Polytetrafluoroethylene
QAS: Quality Audit Standard
Qs Quantum Satis (amount which is enough)
RC: Related Compound
RRT: Relative Retention Time
RSD: Relative Standard Deviation
RT: Retention Time
SUPACC: Scale-Up and Post-Approval Changes
SVF: Simulated Vaginal Fluid
UNK: Unknown
USP: United States Pharmacopeia
UV-Vis: Ultraviolet/Visible Light
VDC: Vertical Diffusion Cell
l/s: Reciprocal Seconds
AE adverse event
AUC0-24 area under the plasma concentration-time curve from time 0 to 24 hour; calculated using the linear/log trapezoid rule (oral dosing only)
AUC LAST area under the plasma concentration-time curve from time 0 (Day 1) to the time of the last quantifiable concentration on Day 5, post single dosing; calculated using the linear/log trapezoid rule (IVR dosing only)
BMI body mass index
BP blood pressure
bpm beats per minute
CFR Code of Federal Regulations
Cavg time-weighted average observed plasma concentration over 24 hours (Day 1 PK profile for single dose)
Cmax maximum observed plasma concentration
conmed concomitant medication
CRU clinical research unit
CRO contract research organization
ECG Electrocardiogram
eCRF electronic case report form
EOS end-of-study
FDA Food and Drug Administration
FSH follicle-stimulating hormone
GCP Good Clinical Practice
HIPAA Health Insurance Portability and Accountability Act of 1996
HIV human immunodeficiency virus
HRT hormone replacement therapy
ICF informed consent form
ICH International Council for Harmonisation
ID Identification
IEC independent ethics committee
IRB institutional review board
LC/MS/MS liquid chromatography/tandem mass spectrometry
MedDRA Medical Dictionary for Regulatory Activities
OTC over-the-counter
Pap Papanicolaou
PK pharmacokinetic(s)
PR pulse rate
QD once daily
RR respiration rate
SAE serious adverse event
SOP standard operating procedure
SRM study reference manual
SSP study-specific procedure
t½, eff effective half-life; calculated using AUC0-24 on Days 1 and 5 (single dose)
tmax time at which the maximum plasma concentration was observed
temp Temperature
ULN upper limit of normal
VVA vulvovaginal atrophy

1. A composition, comprising:
a poloxamer;
a stabilization polymer; and
an active ingredient for treating dysmenorrhea,
wherein the composition has a viscosity at room temperature of between about 800,000 centipoise (cP) and about 1.8 million cP.

2. The composition of claim 1, where the composition is a gel.

3. The composition of claim 1, wherein the composition has a viscosity at 5° C. of between about 2,000 centipoise (cP) and about 220,000 cP.

4. The composition of claim 1, wherein the composition has a viscosity at 37° C. of between about 800,000 centipoise (cP) and about 1.3 million cP.

5. The composition of claim 1, wherein the composition contains no other component that changes the viscosity of said composition at room temperature by more than +/−100,000 centipoise.

6. The composition of claim 1, wherein the poloxamer is present at 10-20 wt %.

7. The composition of claim 1, wherein the poloxamer comprises poloxamer 407.

8-16. (canceled)

17. The composition of claim 1, wherein the composition comprises air at no more than 15 vol %.

18-20. (canceled)

21. The composition of claim 1, wherein the composition has a density of about 1 g/cm³.

22. The composition of claim 1, wherein the active ingredient comprises diclofenac and/or a pharmaceutically acceptable salt thereof.

23. The composition of claim 22, wherein the active ingredient comprises diclofenac potassium.

24. The composition of claim 1, wherein the active ingredient is present at 1-15 wt %.

25-36. (canceled)

37. A method for treating dysmenorrhea or symptom thereof, comprising:
applying, to a vagina of a subject, a composition comprising a poloxamer, a stabilization polymer, and an active ingredient for treating dysmenorrhea, wherein the composition, as applied, has a viscosity of at least 800,000 cP.

38. The method of claim 37, wherein the active ingredient comprises diclofenac or pharmaceutically acceptable salt thereof.

39. The method of claim 37, wherein the subject has dysmenorrhea.

40-42. (canceled)

43. The method of claim 37, where the composition is a gel.

44. The method of claim 37, wherein the poloxamer comprises poloxamer 407.

45-46. (canceled)

47. The method of claim 37, comprising applying the composition through an applicator to the vagina.

48. A method, comprising:
providing a composition comprising a poloxamer, a stabilization polymer, and diclofenac or pharmaceutically acceptable salt thereof; and
removing air from the composition.

49. The method of claim 48, further comprising filling an applicator with the composition.

50. The method of claim 48, wherein removing air comprises exposing the composition to a pressure of less than 100 mbar (absolute).

51-55. (canceled)

56. The method of claim 48, comprising removing air such that the composition comprises no more than 15 vol % air.

57. The method of claim 48, wherein the composition has a viscosity at room temperature of at least 800,000 cP.

58. The method of claim 48, wherein the poloxamer comprises poloxamer 407.

59. The method of claim 48, wherein the stabilization polymer comprises xanthan gum.

60-101. (canceled)
